SEQUENCE LISTING (24 sequences)

SEQ ID NO: 1 - DNA, 770 bp, Artificial Sequence
  PTCH - patched homolog of Drosophila
taaacgtaaaaagtagccaagcgcacgggggaagggccccggccggcgcaggcaggggtcccggntgggctgcggctgatcccggcngcngcgtgatctcggcgctggccgcatgccccggcgggnccccgtctgggtgctcgccttccccggattccacncattgcagcgagcctcgtaaacncaatgaanccggccgcttggcagacccgcaccgcgganttaangtggcaatttgtttacnnctttccctctccccccaggctctgggaagaggngactcaaaaactgaaaaggaagaggggagatgccctctttnaaggataatttttaagggggnnganatttcnagctcagcaaaagcaaaaccggatgccaaaaaaggaaaccacctttatttcngctncctcccccccttccatctctccgcctctctccactccgctttccnccctcaaaagatgttaaaaaaatgtggcagcatttcncgggnnttgggacngcaaantaaggngccaaggggctangnccatctggggttctccnngggcncgggtntnccgggtcgntgacctcgcggactgtntggcnntcntagnatggcncccgcanaancgctntncantnntctgtnaaaaggnatnncttttaancntccttacnacccntccnaccncacccaaatnanntttnttcttgnatatgctgatnnatcncttgccgatttcttaancntcttncctacccntgnnncaagggnaggtatannt

SEQ ID NO: 2 - DNA, 694 bp, Artificial Sequence
  PSMD2 - proteasome (prosome, macropain) 26S subunit, non-ATPase2
cttcttcntgactcctggatttcctctgttcncaacgggacacagccttaccaaattcaaacggccgagaggacgttatgtatcatctagaactaatcctgacttcaacagtgtccttcacaccccttctaagtcaaatcacggaaagactcaaaagacagagattgaagaaggcaaagcctgtgtcttgatctgcctttagttctagagtttagcatcngagcatangaccacattgtattgatggactccgaccaggntccgcaggnggatttaaggtgggggccgtacgcggcaggtggtacccgaccactctccttcaccnnggggtaaaacgttacgaggttaatattccgcggcggcggaagtagatacaggttgcagatctcacacgggcggcgatcaagcattccgaagagtctcgttcgtctgtcccaccacgcagccgactgcggtgtcactgtgggtaccggtcgctcggcnagtaaggagaccccgcgggcggnccctcggntcgcggctcttcatctcctaccgcagccagcggactcggatcncagactgcacggccncatggccttccggaaactcccggtccgagccggggcggcgcctggggcgnatnaacngttagaacttgcagttttgggggcggnctccgagggngggggtccagggcccgggcctcncgaaa

SEQ ID NO: 3 - DNA, 780 bp, Artificial Sequence
  NMT 1 - N-myristoyltransferase 1
gtctccagtttagggaaccatgggggaaggaagaaaagtcgcgcantatcatgccatcctgcgtttgcgcnaatggatgggtgggaatcccatgctgccacnnangnccgggggaaaagaggtgttttctcttaaaattttntanccggtcnagccnctggggaaaatgtaaggggaggcnaagccttctgaaaagtggagatgatnactcagcgaaacaaaagtacncattnaancacttttaattcactctatganataggtaccattcccgntttccagatgagcaaactgagagtcagaaaggtacgcaagttgacngaaatggaaaggncnnatgttagatncaaaaataaangagatctgggcagcggtggntcagcgncttancgccgccttnagcccagggcatgatcctggggtcccgggatcgagtcccacgtcgggctccctgcatggagcctgcttctccctctgcctgtgtctctctctgngnctatcangaaataaataagntnntaanatatcanatnttaaaaaaatnntctccctcagnatctgccccccnnagtttcttgagtcctagnggncttttggnactggaacctgcctgtatcttcaacccacctttctcaaatcnnnagntgnaaannaggnaanggaacncctncctnaaccgggtgccnttnagggctgatgacccacngtattccaggcnnttttacccangggnttgnntccaaanatccntgctccaacaattnnantnaaaggnttgaa

SEQ ID NO: 4 - DNA, 620 bp, Artificial Sequence
  MARCO - macrophage receptor with collagenous structure
ctggtgctgccctctcttccacccactcactcacctttctctggtcatcttgaattcctacagtttatcaatgctgttccttcaattgaacgacttctctcactcccaaatcccttctggtgaatgactatcactcatcctaagggcaccttttcaatgaatcctactgccaagtagaactgacccctcacactcccaatccatcttttcaatgtatattctgcacagagattcctcaatagcacaaataactctacaagttggttgttttttctttctttttttagagattttatttaagaaagagagagagagaacacaagagggagggagaggcaacaagagaggaaaaaacagattccctgctgaacagggagctcaaagcggggctcagtcttagtaccctgagaccatgacctgaacagaaggcagatggttaactgaatgagccaccgaggtgccccagtggttgcttttattggtctcttcccgactgtgagttccccaagagcaggaaccacacattacattgcttaaacctcagttcaagcaggaataaagaagngaaaggatgatggnaattatccaaacnctgaggagcaaaccccacgcancatgcc

SEQ ID NO: 5 - DNA, 700 bp, Artificial Sequence
  DCK6 - cyclin-dependent kinase
cctctgcctatgtctctgcctctctctctctctctctctctctgtgactatcataaataaataaaaattaaaaaaaaaaaagatattcagttctgatctgtgtcagattcaccgtgaagtgttctcttttaaataaataaataaataaataaataaataagtaagtaagtaaataaagcgctaaacataacaggaaagattggccatacagacttcttacaatttaaaacgtcttttcatgggacacctgaatggctcaatgttggacatccgaccctcaattttggctcaggttatgatctcggggtcatgggatcaagtcccactagacacagtctgcttgttcttctccctctgctcctcctcaattctctctctctttctcaaatgaataaataaaatctttaaaaaaataaaacctctattcatcaaaatataacattaagagaatgaaaagacnagaagtaatgtggaataagacattttacatggataaatcatncnaaggactatttctagaccatataaatatctcttanaaattaataagnnnaaattgtctgactcaattatttttaagagnaggataaaaganttgaatagattttttncaaatgaaaatatcccaatggnccaatgnccatgaaaatatnntccnnccncnaaagntatccggaaaatgcnagnnggaaattaaacn

SEQ ID NO: 6 - DNA, 690 bp, Artificial Sequence
  FLJ16046 - MDCK gent (Madin Darby Canine Kidney)
tgatctccagatttacatattcagttcctacttgacaactccccttggatatttcaaagatatctcaaattcaaagtgtcacacctgtcacacactcttctgctctctgccccttcaacctgatcctctctttttttngactctatgaaaggcatcncctttcattctatttagctagagactanaaggcactctagcattctttctctaccccttacccaattgattacctaatcccatggatttcacctccttaaatatctctgtcatctcttgcttcccttgtcccactttatcttcaccacctccacctcccgccatccagagaaattagtcatccagctagtttccttatatttacctttatactcctttcctgcattagncatatgaaagccacaatgatttctaacaagatactaatctgatatcctgttaaactccttcntaaaaaactttagtggcttaccttcagtcttaagatagaaaatataacttctaagaaggacccacatggntcctcaaggactagttctcctgacctctccattctcatcacacaggacttgcccccttgctgtcttctcttcagtcctgcttntgnntcccccagaaattttgtgtatgccaggctcctacatgccaaagagcatttgcaatgctgttccctctgttttagaaaancttata

SEQ ID NO: 7 - DNA, 568 bp, Artificial Sequence
  PCSK6 - proprotein convertase subtilisin/kexin type 6
tgttctatgtattatatagatgaaatatctttcttctatcttccctgaggacaccatatgagataacagaatttatatcctggtctctgttttagttcttggcacanagctcctgagaaccttgtcatttcctgattgggaagagcaataggaggatcttttgttataatatttgcctttgaccctgttcctgactcagtactaacatccttgtaaattcctaagtgataagagcactaggaacatcctttgttctacgaaggggacttggggtgggctcctggatgggggctggtcaccaaaaggaccaagctacgattanaaacttggaattttcagccctgtcccccacttctctanagaggggagaacaatnaagtccnttactgatcatacctacctgaggaagcctccttaaaatcncaatagnnatgaggatctggngagattccnaantgngcnaacncatncnntnccnngagggtgnnnnacccnnncnctgccnggncaganccncctngtnttgnnanctncccntacttaaccnttccnnggaantcntcagagt

SEQ ID NO: 8 - DNA, 565 bp, Artificial Sequence
  PTGDR - prostaglandin D2 receptor (DP)
ggtgccttagacattacaggcggggcaccatgggtggcatcagtggttgagatgactgcctttgactcagggtgtgacccatggggtcctgggatcaagtcctgcatccggctccctgcagggagcccacttctccctcttcctaggtctctgcctctctccttatatctctcatgaataaataaataaaaatctttaaaaaaaattagaggcattatggatggcacgtgatgtgattagcattggattgacaaattgacaaattgaatttaagtaaaaaaaaatacaggnaaaaatgctactgggaggggtgcctgggtcgctctgttggttaaaactttgcctttggctcaggtcatgatctcagggttctgngnattgagccccaccttaggctctgcttgtttctctgcccctccccctgctnnnntttctatcgaataaanaaaanccttaaaaaaaaatgctattgggagttatttgattacctacaagtgaaaagatntgacagtcggagatcanaaaaacattatgtctattacntattttancttttttttttttt

SEQ ID NO: 9 - DNA, 6819 bp, Artificial Sequence
  PTCH - patched homolog of Drosophila
gcgcccgccgtgtgagcagcagcagcggctggtctgtcaaccggagcccgagcccgagcagcctgcggccagcagcgtcctcgcaagccgagcgcccaggcgcgccaggagcccgcagcagcggcagcagcgcgccgggccgcccgggaagcctccgtccccgcggcggcggcggcggcggcggcaacatggcctcggctggtaacgccgccgagccccaggaccgcggcggcggcggcagcggctgtatcggtgccccgggacggccggctggaggcgggaggcgcagacggacgggggggctgcgccgtgctgccgcgccggaccgggactatctgcaccggcccagctactgcgacgccgccttcgctctggagcagatttccaaggggaaggctactggccggaaagcgccgctgtggctgagagcgaagtttcagagactcttatttaaactgggttgttacattcaaaaaaactgcggcaagttcttggttgtgggcctcctcatatttggggccttcgcggtgggattaaaagcagcgaacctcgagaccaacgtggaggagctgtgggtggaagttggaggacgaggtgaattaaattatactcgccagaagattggagaagaggctatgtttaatcctcaactcatgatacagacccctaaagaagaaggtgctaatgtcctgaccacagaagcgctcctacaacacctggactcggcactccaggccagccgtgtccatgtatacatgtacaacaggcagtggaaattggaacatttgtgttacaaatcaggagagcttatcacagaaacaggttacatggatcagataatagaatatctttacccttgtttgattattacacctttggactgcttctgggaaggggcgaaattacagtctgggacagcatacctcctaggtaaacctcctttgcggtggacaaacttcgaccctttggaattcctggaagagttaaagaaaataaactatcaagtggacagctgggaggaaatgctgaataaggctgaggttggtcatggttacatggaccgcccctgcctcaatccggccgatccagactgccccgccacagcccccaacaaaaattcaaccaaacctcttgatatggcccttgttttgaatggtggatgtcatggcttatccagaaagtatatgcactggcaggaggagttgattgtgggtggcacagtcaagaacagcactggaaaactcgtcagcgcccatgccctgcagaccatgttccagttaatgactcccaagcaaatgtacgagcacttcaaggggtacgagtatgtctcacacatcaactggaacgaggacaaagcggcagccatcctggaggcctggcagaggacatatgtggaggtggttcatcagagtgtcgcacagaactccactcaaaaggtgctttccttcaccaccacgaccctggacgacatcctgaaatccttctctgacgtcagtgtcatccgcgtggccagcggctacttactcatgctcgcctatgcctgtctaaccatgctgcgctgggactgctccaagtcccagggtgccgtggggctggctggcgtcctgctggttgcactgtcagtggctgcaggactgggcctgtgctcattgatcggaatttcctttaacgctgcaacaactcaggttttgccatttctcgctcttggtgttggtgtggatgatgtttttcttctggcccacgccttcagtgaaacaggacagaataaaagaatcccttttgaggacaggaccggggagtgcctgaagcgcacaggagccagcgtggccctcacgtccatcagcaatgtcacagccttcttcatggccgcgttaatcccaattcccgctctgcgggcgttctccctccaggcagcggtagtagtggtgttcaattttgccatggttctgctcatttttcctgcaattctcagcatggatttatatcgacgcgaggacaggagactggatattttctgctgttttacaagcccctgcgtcagcagagtgattcaggttgaacctcaggcctacaccgacacacacgacaatacccgctacagccccccacctccctacagcagccacagctttgcccatgaaacgcagattaccatgcagtccactgtccagctccgcacggagtacgacccccacacgcacgtgtactacaccaccgctgagccgcgctccgagatctctgtgcagcccgtcaccgtgacacaggacaccctcagctgccagagcccagagagcaccagctccacaagggacctgctctcccagttctccgactccagcctccactgcctcgagcccccctgtacgaagtggacactctcatcttttgctgagaagcactatgctcctttcctcttgaaaccaaaagccaaggtagtggtgatcttcctttttctgggcttgctgggggtcagcctttatggcaccacccgagtgagagacgggctggaccttacggacattgtacctcgggaaaccagagaatatgactttattgctgcacaattcaaatacttttctttctacaacatgtatatagtcacccagaaagcagactacccgaatatccagcacttactttacgacctacacaggagtttcagtaacgtgaagtatgtcatgttggaagaaaacaaacagcttcccaaaatgtggctgcactacttcagagactggcttcagggacttcaggatgcatttgacagtgactgggaaaccgggaaaatcatgccaaacaattacaagaatggatcagacgatggagtccttgcctacaaactcctggtgcaaaccggcagccgcgataagcccatcgacatcagccagttgactaaacagcgtctggtggatgcagatggcatcattaatcccagcgctttctacatctacctgacggcttgggtcagcaacgaccccgtcgcgtatgctgcctcccaggccaacatccggccacaccgaccagaatgggtccacgacaaagccgactacatgcctgaaacaaggctgagaatcccggcagcagagcccatcgagtatgcccagttccctttctacctcaacggcttgcgggacacctcagactttgtggaggcaattgaaaaagtaaggaccatctgcagcaactatacgagcctggggctgtccagttaccccaacggctaccccttcctcttctgggagcagtacatcggcctccgccactggctgctgctgttcatcagcgtggtgttggcctgcacattcctcgtgtgcgctgtcttccttctgaacccctggacggccgggatcattgtgatggtcctggcgctgatgacggtcgagctgttcggcatgatgggcctcatcggaatcaagctcagtgccgtgcccgtggtcatcctgatcgcttctgttggcataggagtggagttcaccgttcacgttgctttggcctttctgacggccatcggcgacaagaaccgcagggctgtgcttgccctggagcacatgtttgcacccgtcctggatggcgccgtgtccactctgctgggagtgctgatgctggcgggatctgagttcgacttcattgtcaggtatttctttgctgtgctggcgatcctcaccatcctcggcgttctcaatgggctggttttgcttcccgtgcttttgtctttctttggaccatatcctgaggtgtctccagccaacggcttgaaccgcctgcccacaccctcccctgagccaccccccagcgtggtccgcttcgccatgccgcccggccacacgcacagcgggtctgattcctccgactcggagtatagttcccagacgacagtgtcaggcctcagcgaggagcttcggcactacgaggcccagcagggcgcgggaggccctgcccaccaagtgatcgtggaagccacagaaaaccccgtcttcgcccactccactgtggtccatcccgaatccaggcatcacccaccctcgaacccgagacagcagccccacctggactcagggtccctgcctcccggacggcaaggccagcagccccgcagggacccccccagagaaggcttgtggccacccctctacagaccgcgcagagacgcttttgaaatttctactgaagggcattctggccctagcaatagggcccgctggggccctcgcggggcccgttctcacaaccctcggaacccagcgtccactgccatgggcagctccgtgcccggctactgccagcccatcaccactgtgacggcttctgcctccgtgactgtcgccgtgcacccgccgcctgtccctgggcctgggcggaacccccgagggggactctgcccaggctaccctgagactgaccacggcctgtttgaggacccccacgtgcctttccacgtccggtgtgagaggagggattcgaaggtggaagtcattgagctgcaggacgtggaatgcgaggagaggccccggggaagcagctccaactgagggtgattaaaatctgaagcaaagaggccaaagattggaaaccccccacccccacctctttccagaactgcttgaagagaactggttggagttatggaaaagatgccctgtgccaggacagcagttcattgttactgtaaccgattgtattattttgttaaatatttctataaatatttaagagatgtacacatgtgtaatataggaaggaaggatgtaaagtggtatgatctggggcttctccactcctgccccagagtgtggaggccacagtggggcctctccgtatttgtgcattgggctccgtgccacaaccaagcttcattagtcttaaatttcagcatatgttgctgctgcttaaatattgtataatttacttgtataattctatgcaaatattgcttatgtaataggattattttgtaaaggtttctgtttaaaatattttaaatttgcatatcacaaccctgtggtagtatgaaatgttactgttaactttcaaacacgctatgcgtgataatttttttgtttaatgagcagatatgaagaaagcacgttaatcctggtggcttctctaggtgtcgttgtgtgcggtcctcttgtttggctgtgcgtgtgaacacgtgtgtgagttcaccatgtactgtactgtgattttttttttgtcttgttttgtttctctacactgtctgtaacctgtagtaggctctgacctagtcaggctggaagcgtcaggatatcttttcttcgtgctggtgagggctggccctaaacatccacctaatcctttcaaatcagcccggcaaaagctagactctcctcgtgtctacggcatctcttatgatcattggctgccatccaggaccccaatttgtgcttcagggggataatctccttctctcggatcattgtgatggatgctggaacctcagggtatggagctcacatcagttcatcatggtgggtgttagagaattcggtgacatgcctagtgctgagccttggctgggccatgagagtctgtatactctaaaaagcatgcagcatggtgcccctcttctgaccaacacacacacgacccctcccccaacacccccaaattcaagagtggatgtggccctgtcacaggtagaaaaacctatttagttaattctttcttggcccacagtctcccagaaatgatgttttgagtccctatagtttaaactccctctcttaaatggagcagctggttgaggctttctagatctgtttgcatcttctttaaaactaagtggtgagcatgcattgtggtgtagaggcaggcattatgtaggataagagctccggggggattcttcatgcaccagtgtttagggtacgtgcttcctaagtaaatccaaacattgtctccatcctccccgtcattagtgctctttcaatgtgatgtgggaaagcaggaggatggacacaccccactgaaagatgtaggcaggggcaggtctctcaaccaggcatatttttaaaagttgcttctgtactggttctcttcttttgctctgaggtgtgggctccctcatctcgtaaccagagaccagcacatgtcagggaagcacccagtgtcggctccccatccaaatccacaccagcaccttgttacagacaagaagtcagaggaaagggcggggtccctgcagggctgaagcctaagctactgtgaggcgctcacgagtggcagctcctgttactcccttttaaattacctgggaaatcttaacagaaaggtaatgggcccccagaaatacccacagcatagtgacctcagaccctgatactcaccacaaaacttttaagatgctgattgggagccgcttgtggctgctgggtgtgtgtgtgtgtgtgtgcgtgcgtgcgtgtgtgtgtgtctctgctggggaccctggccacccccctgctgctgtcttggtgcctgtcacccacatggtctgccatcctaacacccagctctgctcagaaaacgtcctgcgtggaggagggatgatgcagaattctgaagtcgacttccctctggctcctggcgtgccctcgctcccttcctgagcccagctcgtgttgcgccggaggctgcgcggcccctgatttctgcatggtgtagaactttctccaatagtcacattggcaaagggagaactggggtgggcggggggtggggctggcagggaattagaatttctctctctcttttaatagttttattttgtctgtcctgtttgttcatttggatgttttaatttttaaaaaaaaaaaaaaaaaa

SEQ ID NO: 10 - DNA, 2990 bp, Artificial Sequence
  PSMD2 - proteasome (prosome, macropain) 26S subunit, non-ATPase2
tgcgcgcgcagcgggccggcagtggcggcggagatggaggagggaggccgggacaaggcgccggtgcagccccagcagtctccagcggcggcccccggcggcacggacgagaagccgagcggcaaggagcggcgggatgccggggacaaggacaaagaacaggagctgtctgaagaggataaacagcttcaagatgaactggagatgctcgtggaacgactaggggagaaggatacatccctgtatcgaccagcgctggaggaattgcgaaggcagattcgttcttctacaacttccatgacttcagtgcccaagcctctcaaatttctgcgtccacactatggcaaactgaaggaaatctatgagaacatggcccctggggagaataagcgttttgctgctgacatcatctccgttttggccatgaccatgagtggggagcgtgagtgcctcaagtatcggctagtgggctcccaggaggaattggcatcatggggtcatgagtatgtcaggcatctggcaggagaagtggctaaggagtggcaggagctggatgacgcagagaaggtccagcgggagcctctgctcactctggtgaaggaaatcgtcccctataacatggcccacaatgcagagcatgaggcttgcgacctgcttatggaaattgagcaggtggacatgctggagaaggacattgatgaaaatgcatatgcaaaggtctgcctttatctcaccagttgtgtgaattacgtgcctgagcctgagaactcagccctactgcgttgtgccctgggtgtgttccgaaagtttagccgcttccctgaagctctgagattggcattgatgctcaatgacatggagttggtagaagacatcttcacctcctgcaaggatgtggtagtacagaaacagatggcattcatgctaggccggcatggggtgttcctggagctgagtgaagatgtcgaggagtatgaggacctgacagagatcatgtccaatgtacagctcaacagcaacttcttggccttagctcgggagctggacatcatggagcccaaggtgcctgatgacatctacaaaacccacctagagaacaacaggtttgggggcagtggctctcaggtggactctgcccgcatgaacctggcctcctcttttgtgaatggctttgtgaatgcagcttttggccaagacaagctgctaacagatgatggcaacaaatggctttacaagaacaaggaccacggaatgttgagtgcagctgcatctcttgggatgattctgctgtgggatgtggatggtggcctcacccagattgacaagtacctgtactcctctgaggactacattaagtcaggagctcttcttgcctgtggcatagtgaactctggggtccggaatgagtgtgaccctgctctggcactgctctcagactatgttctccacaacagcaacaccatgagacttggttccatctttgggctaggcttggcttatgctggctcaaatcgtgaagatgtcctaacactgctgctgcctgtgatgggagattcaaagtccagcatggaggtggcaggtgtcacagctttagcctgtggaatgatagcagtagggtcctgcaatggagatgtaacttccactatccttcagaccatcatggagaagtcagagactgagctcaaggatacttatgctcgttggcttcctcttggactgggtctcaaccacctggggaagggtgaggccatcgaggcaatcctggctgcactggaggttgtgtcagagccattccgcagttttgccaacacactggtggatgtgtgtgcatatgcaggctctgggaatgtgctgaaggtgcagcagctgctccacatttgtagcgaacactttgactccaaagagaaggaggaagacaaagacaagaaggaaaagaaagacaaggacaagaaggaagcccctgctgacatgggagcacatcagggagtggctgttctggggattgcccttattgctatgggggaggagattggtgcagagatggcattacgaacctttggccacttgctgagatatggggagcctacactccggagggctgtacctttagcactggccctcatctctgtttcaaatccacgactcaacatcctggataccctaagcaaattctctcatgatgctgatccagaagtttcctataactccatttttgccatgggcatggtgggcagtggtaccaataatgcccgtctggctgcaatgctgcgccagttagctcaatatcatgccaaggacccaaacaacctcttcatggtgcgcttggcacagggcctgacacatttagggaagggcacccttaccctctgcccctaccacagcgaccggcagcttatgagccaggtggccgtggctggactgctcactgtgcttgtctctttcctggatgttcgaaacattattctaggcaaatcacactatgtattgtatgggctggtggctgccatgcagccccgaatgctggttacgtttgatgaggagctgcggccattgccagtgtctgtccgtgtgggccaggcagtggatgtggtgggccaggctggcaagccgaagactatcacagggttccagacgcatacaaccccagtgttgttggcccacggggaacgggcagaattggccactgaggagtttcttcctgttacccccattctggaaggttttgttatccttcggaagaaccccaattatgatctctaagtgaccaccaggggctctgaactgcagctgatgttatcagcaggccatgcatcctgctgccaagggtggacacggctgcagacttctgggggaattgtcgcctcctgctcttttgttactgagtgagataaggttgttcaataaagacttttatccccaaggaaaaaaaaaaaaaaaaaaaaaaaaaaaaaaaaaaaaaaaaaaaaaaaaaaaaaaaaaaaaaaaaaaaa

SEQ ID NO: 11 - DNA, 4903 bp, Artificial Sequence
  NMT 1 - N-myristoyltransferase 1
ctgctctcgcaactcaagatggcggacgagagtgagacagcagtgaagccgccggcacctccgctgccgcagatgatggaagggaacgggaacggccatgagcactgcagcgattgcgagaatgaggaggacaacagctacaaccggggtggtttgagtccagccaatgacactggagccaaaaagaagaaaaagaaacaaaaaaagaagaaagaaaaaggcagtgagacagattcagcccaggatcagcctgtgaagatgaactctttgccagcagagaggatccaggaaatacagaaggccattgagctgttctcagtgggtcagggacctgccaaaaccatggaggaggctagcaagcgaagctaccagttctgggatacgcagcccgtccccaagctgggcgaagtggtgaacacccatggccccgtggagcctgacaaggacaatatccgccaggagccctacaccctgccccagggcttcacctgggatgctttggacttgggcgatcgtggtgtgctaaaagaactgtacaccctcctgaatgagaactatgtggaagatgatgacaacatgttccgatttgattattccccggagtttcttttgtgggctctccggccacccggctggctcccccagtggcactgtggggttcgagtggtctcaagtcggaaattggttgggttcattagcgccatcccagcaaacatccatatctatgacacagagaagaagatggtagagatcaacttcctgtgtgtccacaagaagctgcgttccaagagggttgctccagttctgatccgagagatcaccaggcgggttcacctggagggcatcttccaagcagtttacactgccggggtggtactaccaaagcccgttggcacctgcaggtattggcatcggtccctaaacccacggaagctgattgaagtgaagttctcccacctgagcagaaatatgaccatgcagcgcaccatgaagctctaccgactgccagagactcccaagacagctgggctgcgaccaatggaaacaaaggacattccagtagtgcaccagctcctcaccaggtacttgaagcaatttcaccttacgcccgtcatgagccaggaggaggtggagcactggttctacccccaggagaatatcatcgacactttcgtggtggagaacgcaaacggagaggtgacagatttcctgagcttttatacgctgccctccaccatcatgaaccatccaacccacaagagtctcaaagctgcttattctttctacaacgttcacacccagacccctcttctagacctcatgagcgacgcccttgtcctcgccaaaatgaaagggtttgatgtgttcaatgcactggatctcatggagaacaaaaccttcctggagaagctcaagtttggcataggggacggcaacctgcagtattacctttacaattggaaatgccccagcatgggggcagagaaggttggactggtgctacaataaccagtcaccagtgcgattctggataaagccactgaaaattcgaaccaggaaatggaaccccaccactgttggtccaattttcacacacgtgagaatccctggcaaagggagcagaactgaaccggctttaccaaaccgccagcgaacttgacaattgtattgcgatggcgtgggctgcgtgacgtcacctccggtcgtgtctctggtctccgtgttttccagttaattacatcctcatgcagccgtgatcaagggaatgtaactgctgaaaactagctcgtgattggcatataatggagttaacgggtgaataataaaagtatatatatatattatatatatataaatattttaaatatctttcatgttccaaatgtacaaggatgtttggtctttaatgaaaagctgaatctagatcattcctcagaatgaggacccgaggacagtggcagacagacgcgttggcacagttcatggtttcctccagaggagacattggcttatcatggggaaaaagaggatctggagaacctcatccagctccccttctgaatcagctgggatgactggctttgagaaggaagggaagatggaacaggctcagatctcatgggatagcacgtggagctcttggctggggctgaccctgggcagggactttcctgcagggccagacctgcctgcattctgagacaaagcaatggacggtccgcagaagcagacctcattgattgagtcctttcttccatccccttggcctgctccctgtaggaagtcatcctgccaactgatttaaaagggctctttagccagttgttgccaaccttatagggatgagtcccctgtgagattttgcttttccactgcctgggatgatgcagtttgaagaggcccttggacctccttgtaacatcagggacctttggagaccattatcagtgtaagccctgcttagctcatcttagagcaaagagccagcaccctgatgtccctggggtggctaggcaggagtggcgtggggccaatacccagaccccttcagccaccagcccctggcctgtgccttccaacccattagccatttcttgttgtgcccctttccaagatacagcctgcaagtggtagcaagaagtgattagaggcagatctggacttggcaacagaagtggtttcccatctccattgtctgagtctgattttcgctgatgctgttttgtggatttttgtggtagtgatggttgtcagtgctgccagtttcccaaaacgtaatcaagcctctggtcacatggctgtcgatgtaggcattctggagtggtgttcagccaagtgaccgggcaaaattgggctgtgaaattgtacttccaggcttggatgtaatttttgctctagagagaagcaagtggtgggaaggaggtagcatgacgtgtggtgtgcgggtttccttgctgccgtcacctctccgctcatacaggaatgaagccttagccaggaggccaggctcagccctgtgccactcaccgaagccactttctacaggccagcaggggcttgttgcaggctgtgggttttggtgtggtttgtcagaggctaattctgcagagtttccaaaaccagaagacatcgtatgcttgggatgggggccgtgccacccgtgggaatgctgcccgctctgcagactgctgctagagccagcaactccactaaggtggattttcatcaggggcctgcagggccctcccttttcccattgttcctgcgctgcaaattgcaggccccagcaatcgtgactgacgtttgctccttgactccaagaaactgagaccaaagaagctgctgttcttagcaagatgcgcactgcattccacaggtgggaggagtcggagaggcaggggcttgctttgcagccccacagacaacagttgcacagtgcctcaagccccagagtggctcaccctgtccagacctttgaggatatcaaaggacaaagtgcccaagtctttcctaccttgggggaacctggaacttggaaaggctccctgtcctagtcttgatctgttctgggccaggtcccagcttgagctgcctctgagatttgggctgtgcggatctctggagtgagctctgtttcggttgacccaggtcatggaatggaaacggtgaggccccagtggctgttctggaagaaacagatctcctggcaaaggccccagcatctccctcactgaaaccaggtggccggctcctcggactctgctttatgttgcggtgagaactctgcccaggtgtgcagggtttggcttgtgggctgcttgctgctcatctgatttttgtcccagtagtccctgcgttcttcattcaaccccttctgggacttcagctcagagagcaccatcccgggggtcagggcctccccacaggagccctgcagtgtggtagcgccatggctgtctcaaaccaagcaaaggaaggaccctgaggccttcacgctaaccatcctcgagcaactgctgttggaaggcctccctgggcctggcccccaccctctgccacccagtcctcccagctgccatgtttcaaagacgacctttacctcctgcctttggattgactctgcatttgaccacggactccagtctgtgtgtagggagagagctgagtaggaggcctccactccggatcgaggcctgtatagggctcgtttccccacacatgcctatttctgaagaggcttctgtcttatttgaaggccagcccacacccagctactttaacaccaggtttatggaaaatgtcaggccttccccacaactcctgtctaactgctgtcgcccccctacttgctggctctcagaagcctaggggagtccctgtggtcctgaattctttccccaaagacgaccagcatttaaccaacctaagggcccaaaggccttggacaactgcatggagctgcactctaggagaaggaggggaaccagatgttagatcaggggagggagcaggagtgtccctcccgtcagtgcctacccacctgtgaggcagccttctgatggcctggcccaccttccccagaaccaggggaggcctgaggcttcagttttactctgctgcaaaatgaaggcgggcctgcaagccgactacacctacggaggctgttgaggacaatttcattccattaaattaaaaaatactgactggctggcaggcaggtgccatgtctgggaacagggacgggggagcttcacctttttgtcttggcttttctttgggctgtgggggggcatccatttccagggtcggggaggaaataccaaatgcattgttgttctgctcaatacatctcacttgtttctaataaagaaagcagctgaacaaaaaaaaaaaaaaaaaaaaa

SEQ ID NO: 12 - DNA, 1863 bp, Artificial Sequence
  MARCO - macrophage receptor with collagenous structure
gggggccaaagggaagtgctgcgaggtttacaaccagctgcagtggttcgatgggaaggatctttctccaagtggttcctcttgaggggagcatttctgctggctccaggactttggccatctataaagcttggcaatgagaaataagaaaattctcaaggaggacgagctcttgagtgagacccaacaagctgcttttcaccaaattgcaatggagcctttcgaaatcaatgttccaaagcccaagaggagaaatggggtgaacttctccctagctgtggtggtcatctacctgatcctgctcaccgctggcgctgggctgctggtggtccaagttctgaatctgcaggcgcggctccgggtcctggagatgtatttcctcaatgacactctggcggctgaggacagcccgtccttctccttgctgcagtcagcacaccctggagaacacctggctcagggtgcatcgaggctgcaagtcctgcaggcccaactcacctgggtccgcgtcagccatgagcacttgctgcagcgggtagacaacttcactcagaacccagggatgttcagaatcaaaggtgaacaaggcgccccaggtcttcaaggccacaagggggccatgggcatgcctggtgcccctggcccgccgggaccacctgctgagaagggagccaagggggctatgggacgagatggagcaacaggcccctcgggaccccaaggcccaccgggagtcaagggagaggcgggcctccaaggaccccagggtgctccagggaagcaaggagccactggcaccccaggaccccaaggagagaagggcagcaaaggcgatgggggtctcattggcccaaaaggggaaactggaactaagggagagaaaggagacctgggtctcccaggaagcaaaggggacaggggcatgaaaggagatgcaggggtcatggggcctcctggagcccaggggagtaaaggtgacttcgggaggccaggcccaccaggtttggctggttttcctggagctaaaggagatcaaggacaacctggactgcagggtgttccgggccctcctggtgcagtgggacacccaggtgccaagggtgagcctggcagtgctggctcccctgggcgagcaggacttccagggagccccgggagtccaggagccacaggcctgaaaggaagcaaaggggacacaggacttcaaggacagcaaggaagaaaaggagaatcaggagttccaggccctgcaggtgtgaagggagaacaggggagcccagggctggcaggtcccaagggagcccctggacaagctggccagaagggagaccagggagtgaaaggatcttctggggagcaaggagtaaagggagaaaaaggtgaaagaggtgaaaactcagtgtccgtcaggattgtcggcagtagtaaccgaggccgggctgaagtttactacagtggtacctgggggacaatttgcgatgacgagtggcaaaattctgatgccattgtcttctgccgcatgctgggttactccaaaggaagggccctgtacaaagtgggagctggcactgggcagatctggctggataatgttcagtgtcggggcacggagagtaccctgtggagctgcaccaagaatagctggggccatcatgactgcagccacgaggaggacgcaggcgtggagtgcagcgtctgacccggaaaccctttcacttctctgctcccgaggtgtcctcgggctcatatgtgggaaggcagaggatctctgaggagttccctggggacaactgagcagcctctggagaggggccattaataaagctcaacatcaaaaaaaaaaaagaaaaaaaaaaaaaaaaa

SEQ ID NO: 13 - DNA, 11609 bp, Artificial Sequence
  CDK6 - cyclin-dependent kinase
ggcttcagccctgcagggaaagaaaagtgcaatgattctggactgagacgcgcttgggcagaggctatgtaatcgtgtctgtgttgaggacttcgcttcgaggagggaagaggagggatcggctcgctcctccggcggcggcggcggcggcgactctgcaggcggagtttcgcggcggcggcaccagggttacgccagccccgcggggaggtctctccatccagcttctgcagcggcgaaagccccagcgcccgagcgcctgagccggcggggagcaagtaaagctagaccgatctccggggagccccggagtaggcgagcggcggccgccagctagttgagcgcaccccccgcccgccccagcggcgccgcggcgggcggcgtccaggcggcatggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctgaggcctcagcagccgccttaagctgatcctgcggagaacacccttggtggcttatgggtccccctcagcaagccctacagagctgtggaggattgctatctggaggccttccagctgctgtcttctggacaggctctgcttctccaaggaaaccgcctagtttactgttttgaaatcaatgcaagagtgattgcagctttatgttcatttgtttgtttgtttgtctgtttgtttcaagaacctggaaaaattccagaagaagagaagctgctgaccaattgtgctgccatttgatttttctaaccttgaatgctgccagtgtggagtgggtaatccaggcacagctgagttatgatgtaatctctctgcagctgccgggcctgatttggtacttttgagtgtgtgtgtgcatgtgtgtgtgtgtgtgtgtgtgtgtgtgtgtgtatgtgagagattctgtgatcttttaaagtgttactttttgtaaacgacaagaataattcaattttaaagactcaaggtggtcagtaaataacaggcatttgttcactgaaggtgattcaccaaaatagtcttctcaaattagaaagttaaccccatgtcctcagcatttcttttctggccaaaagcagtaaatttgctagcagtaaaagatgaagttttatacacacagcaaaaaggagaaaaaattctagtatattttaagagatgtgcatgcattctatttagtcttcagaatgctgaatttacttgttgtaagtctattttaaccttctgtatgacatcatgctttatcatttcttttggaaaatagcctgtaagctttttattacttgctataggtttagggagtgtacctcagatagattttaaaaaaaagaatagaaagcctttatttcctggtttgaaattcctttcttccctttttttgttgttgttattgttgtttgttgttgttattttgtttttgtttttaggaatttgtcagaaactctttcctgttttggtttggagagtagttctctctaactagagacaggagtggccttgaaattttcctcatctattacactgtactttctgccacacactgccttgttggcaaagtatccatcttgtctatctcccggcacttctgaaatatattgctaccattgtataactaataacagattgcttaagctgttcccatgcaccacctgtttgcttgctttcaatgaacctttcataaattcgcagtctcagcttatggtttatggcctcgattctgcaaacctaacagggtcacatatgttctctaatgcagtccttctacctggtgtttacttttgctacccaaataatgagtaggatcttgttttcgtatacccccaccactcccattgctaccaactgtcaccttgtgcactccttttttatagaagatattttcagtgtctttacctgagggtatgtctttagctatgttttagggccatacatttactctatcaaatgatcttttctccatcccccaggctgtgcttatttctagtgccttgtgctcactcctgctctctacagagccagcctggcctgggcattgtaaacagcttttcctttttctcttactgttttctctacagtcctttatatttcataccatctctgccttataagtggtttagtgctcagttggctctagtaaccagaggacacagaaagtatcttttggaaagtttagccacctgtgctttctgactcagagtgcatgcaacagttagatcatgcaacagttagattatgtttagggttaggattttcaaagaatggaggttgctgcactcagaaaataattcagatcatgtttatgcattattaagttgtactgaattctttgcagcttaatgtgatatatgactatcttgaacaagagaaaaaactaggagatgtttctcctgaagagcttttggggttgggaactattcttttttaattgctgtactacttaacattgttctaattcagtagcttgaggaacaggaacattgttttctagagcaagataataaaggagatgggccatacaaatgttttctactttcgttgtgacaacattgattaggtgttgtcagtactataaatgcttgagatataatgaatccacagcattcaaggtcaggtctactcaaagtctcacatggaaaagtgagttctgcctttcctttgatcgagggtcaaaatacaaagacatttttgctagggcctacaaattgaatttaaaaactcactgcactgattcatctgagctttttggttagtattcatggctagagtgaacatagctttagtttttgctgttgtaaaagtgttttcataagttcactcaagaaaaatgcagctgttctgaactggaatttttcagcattctttagaattttaaatgagtagagagctcaacttttattcctagcatctgcttttgactcatttctaggcagtgcttatgaagaaaaattaaagcacaaacattctggcattcaatcgttggcagattatcttctgatgacacagaatgaaagggcatctcagcctctctgaactttgtaaaaatctgtccccagttcttccatcggtgtagttgttgcatttgagtgaatactctcttgatttatgtattttatgtccagattcgccatttctgaaatccagatccaacacaagcagtcttgccgttagggcattttgaagcagatagtagagtaagaacttagtgactacagcttattcttctgtaacatatggtttcaaacatctttgccaaaagctaagcagtggtgaactgaaaagggcatattgccccaaggttacactgaagcagctcatagcaagttaaaatattgtgacagatttgaaatcatgtttgaatttcatagtaggaccagtacaagaatgtccctgctagtttctgtttgatgtttggttctggcggctcaggcattttgggaactgttgcacagggtgcagtcaaaacaacctacatataaaaattacataaaagaaccttgtccatttagctttcataagaaatcccatggcaaagagtaataaaaaggacctaatcttaaaaatacaatttctaagcacttgtaagaacccagtgggttggagcctcccactttgtccctcctttgaagtggatgggaactcaaggtgcaaagaacctgttttggaagaaagcttggggccatttcagccccctgtattctcatgattttctctcaggaagcacacactgtgaatggcagacttttcatttagccccaggtgacttactaaaaatagttgaaaattattcacctaagaatagaatctcagcattgtgttaaataaaaatgaaagctttagaaggcatgagatgttcctatcttaaataaagcatgtttcttttctatagagaaatgtatagtttgactctccagaatgtactatccatcttgatgagaaaactcttaaatagtaccaaacattttgaactttaaattatgtatttaaagtgagtgtttaagaaactgtagctgcttcttttacaagtggtgcctattaaagtcagtaatggccattattgttccattgtggaaattaaattatgtaagcttcctaatatcataaacatattaaaattcttctaaaatattgcttttcttttaagtgacaatttgactattcttatgataagcacatgagagtgtcttacattttccaaaagcaggctttaattgcatagttgagtctaggaaaaaataatgttaaaagtgaatatgccaccataattacttaattatgttagtatagaaactacagaatatttaccctggaaagaaaatattggaatgttattataaactcttagatatttatataattcaaaagaatgcatgtttcacattgtgacagataaagatgtatgatttctaaggctttaaaaattattcataaaacagtgggcaatagataaaggaaattctggagaaaatgaaggtatttaaagggtagtttcaaagctatatatattttgaaggatatattctttatgaacaaatatattgtaaaaatttatactaaggtcatctggtaactgtgggattaatatggtcgaaaacaaatgttatggagaagctgtcccaagcaaactaaattacctgtacttttttcccatttcaagggaagaggcaaccacatgaagcaatacttcttacacatgcctaagaacgttcattgaaaaaataaatttttaaaaggcatgtgtttcctatgccaccaatacttttgaaaaattgtgaaccttacccaaaaccatttatcatgtccattaagtatatttgggtatataattaggaagatatttacatgttccatctccacagtggaaaaacttattgaggctaccaaagtgtgccaagaaatgtaagtccttagagtaattagaaatgctgttttcctcaaaagcatgagaaactagcattttcatttcttatttactccctttctatatcaatgcaattcacaacccaattttaatacatccctatatctcaagcatttctatcttgtactttttcagaaaataaaccaaaaataatcctttggtctctctatcttctgacctttgtaagcaacagaaatgtaaaaacagaaggggtccaatttttacacgtttttttctcaagtagcctttctggggatttttattttcttaatgaagtgccaatcagcttttcaaaatgttttctatttctcagcatttccaggaagtgataacgtttagctaaatgagtagaagtggacttccttcaacatattgttaccttgtctagccttaggaagaaaacaagagccacctgaaaataaatacaggctcttttcgagcatctgctgaaatactgttacagcaatttgaagttgatgtggtaggaaaggaaggtgacttttcttgcaaaagtctttctaaacattcacactgtcctaagagatgagctttcttgttttattccggtatattccacaaggtggcacttttagagaaaaacaaatctgatgaagactaaagaggtacttctaaaagagatttcattctaactttatttttctgcgcatatttaactctttcctagcacttgttttttgggatgattaatagtctctataatgttctgtaacttcaatattttacttgttacctaggttctgaacaattgtctgcaaataaattgttcttaaggatggataatacacccattttgatcatttaagtaaagaaagcctagtcattcattcagtcaagaaaaaatttttgaagtacccagttaccttacttttctagattaaaacaggcttagttactaaaaaggcagtcctcatctgtgaacaggatagtttcgttagaagtataaaactcctttagtggccccagttaaaacacacataccctctctgctgctttcaaattccctagcatggtggcctttcaacattgattaaattttaaaatcctaatttaaagatcaggtgagcaaaatgagtagcacatcagtaattcagtagacaaaacttttgtctgaaaaattgctgtattgaaacagagccctaaaataccaaaagaccaggtaattttaacatttgtggaatcacaaatgtaaattcataagaagctctaattaaaaaaaaaaagtctgaagtatatgagcataacaacttaggagtgtgtctacatacttaacttttgaagttttttggcaactttatatactttttttaaatttacaagtctacttaaagacttcttataccccaaatgattaagttaattttagaggtcacctttctcacagcagtgtcacttgaaatttagtagggaaggatattgcagtatttttcagtttccttagcacagcaccacagaaagcagcttattccttttgagtggcagacactcgacggtgcctgcccaactttcctcctgagtggcaagcagatgagtctcagtaattcatactgaaccaaaatgccacatacactaggggcagtcagaaactggctgagaaatcccccgcctcattcgcccctctgctcccaggaactagagtccagttaaagcccctatgcgaaaggccgaattccaccccagggtttgttataacagtggccagtctgaaccccatttgctcgtgctcaaaacttgattcccacttgaaagccttccgggcgcgctgcctcgttgccccgcccctttggcaggagagaggcagtgggcgaggccgggctggggccccgcctcccactcacctgccggtgcctgaaattatgtgcggccccgcgggctgctttccgaggtcagagtgccctgctgctgtctcagaggcatctgttctgcaaatcttaggaagaaaaatgtccctagtagcaaacgggtgtcttctgtgcataaataagtacaacacaattctccgaaagttcgggtaaaaagagatgcggtagcagctgccctgtgtgaagctgtctaccccgcatctctcaggcgctaagctcagtttttgtttttgtttttgtttttttaaagaaaagatgtataattgcaggaatttttttttatttttttattttccatcattctatatatgtgatggtgaaagatatgcctggaaaagttttgttttgaaaagtttattttctgcttcgtcttcagttggcaaaagctctcaattctttagcttccagtttcttttctctctttttctttgttaggtaattaaaggtatgtaaacaaattatctcatgtagcaggggattttcatgttgagaggaatcttccgtgtgagttgtttggtcacacaaataaccctttctcaattttaggagtttggattgtcaaatgtaggtttttctcaaagggggcatataactacatattgactgccaagaactatgactgtagcactaatcagcacacatagagccacacaattatttaatttctaactctctgtggtccctagaaaaattccgttgatgtgcttaggttaaagttctgaagatacccgttgtacccttacttgaaagtttctaatcttaagttttatgaaatgcaataatatgtatcagctagcaatatttctgtgatcaccaacaactctcagtttgatcttaaagtctgaataataaaacaaatcccagcagtaatacatttcttaaacctcacagtgcatgatatatcttttcattctgatcctgtgtttgcaaaaatatacacatgtatatcatagttcctcactttttattcatttgttttcctattacctgtagtaaatatattagttagtacatggaatttatagcatcagctacccccaggaacagcacctgacaggcgggggattttttttcaagttgttctacatttgcataaattatttctattattattcatgtatgttatttatttctgaatcacactagtcctgtgaaagtacaactgaaggcagaaagtgttaggattttgcatctaatgttcattatcatggtattgatggacctaagaaaataaaaattagactaagcccccaaataagctgcatgcatttgtaacatgattagtagatttgaatatatagatgtagtattttgggtatctaggtgttttatcattatgtaaaggaattaaagtaaaggactttgtagttgtttttattaaatatgcatatagtagagtgcaaaaatatagcaaaaataaaaactaaaggtagaaaagcattttagatatgccttaatttagaaactgtgccaggtggccctcggaatagatgccaggcagagaccagtgcctgggtggtgcctcctcttgtctgccctcatgaagaagcttccctcacgtgatgtagtgccctcgtaggtgtcatgtggagtagtgggaacaggcagtactgttgagaggagagcagtgtgagagtttttctgtagaagcagaactgtcagcttgtgccttgaggcttccagaacgtgtcagatggagaagtccaagtttccatgcttcaggcaacttagctgtgtacagaagcaatccagtgtggtaataaaaagcaaggattgcctgtataatttattataaaataaaagggattttaacaaccaacaattcccaacacctcaaaagcttgttgcattttttggtatttgaggtttttatctgaaggttaaagggcaagtgtttggtatagaagagcagtatgtgttaagaaaagaaaaatattggtcacgtagagtgcaaattagaactagaaagttttatacgattatcattttgagatgtgttaaagtaggttttcactgtaaaatgtattagtgtttctgcattgccatagggcctggttaaaactttctcttaggtttcaggaagactgtcacatacagtaagcttttttccttctgacttataatagaaaatgttttgaaagtaaaaaaaaaaaatctaatttggaaatttgacttgttagtttctgtgtttgaaatcatggttctagaaatgtagaaattgtgtatatcagatactcatctaggctgtgtgaaccagcccaagatgaccaacatccccacacctctacatctctgtcccctgtatctcttcctttctaccactaaagtgttccctgctaccatcctggcttgtccacatggtgctctccatcttcctccacatcatggaccacaggtgtgcctgtctaggcctggccaccactcccaacttgacctagccacattcatctagagatggttcctgatgctgggcacagactgtgctcatggcacccattagaaatgcctctagcatctttgtatgcatcttgatttttaaaccaagtcattgtacagagcattcagttttggctgtggtaccaagagaaaaactaatcaagaatataaaccacattccaggctgctgttttctctccatctacaggccacacttttactgtatttcttcatacttgaaattcattctgctattttcatatcagggtacagacttataagggtgcatgttccttaaaggtgcataattattcttattccgtttgcttatattgctacagaatgctctgttttggtgctttgagttctgcagacccaagaagcagtgtggaaattcactgcctgggacacagtcttataagaatgttggcaggtgactttgtatcagatgttgcttctcttttctctgtacacagattgagagttaccacagtggcctgtcgggtccaccctgtgggtgcagcacagctctctgaaagcaagaaccttcctacctattctaacgtttttgccctctaagaaaaatggcctcaggtatggtatagacatagcaagaggggaagggctgtctcactctagcaaccatccctccattacacacagaaagccctcttgaagcaaaagaagaagaaagaaagaaagcttatctctaaggctactgtcttcagaatgctctgagctgaatgctcttgctcctttcccaagaggcagatgaaaatatagccagtttatctatacccttcctatctgaggaggagaatagaaaagtagggtaaatatgtaacgtaaaatatgtcattcaaggaccaccaaaactttaagtaccctatcattaaaaatctggttttaaaagtagctcaagtaagggatgctttgtgacccagggtttctgaagtcagatagccattcttacctgccccttactctgacttattgggaaaggagaactgcagtggtgtttctgttgcagtggcaaaggtaacatgtcagaaaattcagagggttgcataccaataatcctttggaaactggatgtcttactgggtgctagaatgaaaatgtaggtatttattgtcagatgatgaagttcattgtttttttcaaaattggtgttgaaatatcactgtccaatgtgttcacttatgtgaaagctaaattgaatgaggcaaaaagagcaaatagtttgtatatttgtaataccttttgtatttcttacaataaaaatattggtagcaaataaaaataataaaaacaataactttaaactgctttctggagatgaattactctcctggctattttcttttttactttaatgtaaaatgagtataactgtagtgagtaaaattcattaaattccaagttttagcagaaaaaaaaaaaaaaaaaaaa

SEQ ID NO: 14 - DNA, 2077 bp, Artificial Sequence
  FLJ16046 - MDCK gene (Madin Darby Canine Kidney)
gatacagatcagatggtgactgaatagaagctgccccagtcctgggctcatgatgtacgcacctgttgaattttcagaagctgaattctcacgagctgaatatcaaagaaagcagcaattttgggactcagtacggctagctcttttcacattagcaattgtagcaatcataggaattgcaattggtattgttactcattttgttgttgaggatgataagtctttctattaccttgcctcttttaaagtcacaaatatcaaatataaagaaaattatggcataagatcttcaagagagtttatagaaaggagtcatcagattgaaagaatgatgtctaggatatttcgacattcttctgtaggcggtcgatttatcaaatctcatgttatcaaattaagtccagatgaacaaggtgtggatattcttatagtgctcatatttcgatacccatctactgatagtgctgaacaaatcaagaaaaaaattgaaaaggctttatatcaaagtttgaagaccaaacaattgtctttgaccttaaacaaaccatcatttagactcacacctattgacagcaaaaagatgaggaatcttctcaacagtcgctgtggaataaggatgacatcttcaaacatgccattaccagcatcctcttctactcaaagaattgtccaaggaagggaaacagctatggaaggggaatggccatggcaggccagcctccagctcatagggtcaggccatcagtgtggagccagcctcatcagtaacacatggctgctcacagcagctcactgcttttggaaaaataaagacccaactcaatggattgctacttttggtgcaactataacaccacccgcagtgaaacgaaatgtgaggaaaattattcttcatgagaattaccatagagaaacaaatgaaaatgacattgctttggttcagctctctactggagttgagttttcaaatatagtccagagagtttgcctcccagactcatctataaagttgccacctaaaacaagtgtgttcgtcacaggatttggatccattgtagatgatggacctatacaaaatacacttcggcaagccagagtggaaaccataagcactgatgtgtgtaacagaaaggatgtgtatgatggcctgataactccaggaatgttatgtgctggattcatggaaggaaaaatagatgcatgtaagggagattctggtggacctctggtttatgataatcatgacatctggtacattgtgggtatagtaagttggggacaatcatgtgcgcttcccaaaaaacctggagtctacaccagagtaactaagtatcgagattggattgcctcaaagaccggtatgtagtgtggattgtccatgagttatacacatggcacacagagctgatactcctgcgtattttgtattgtttaaattcatttactttggattagtgcttttgctagatgtcaagaagcccttcagacccagacaaatctaatatcctgaggtggcctttacatacgtaggaccaaaccctctctaccatgagggaagaagacacagcaaatgacagacagcacctattccttactcacaagggaaactgcttgtgatacttcctaataagataaatgagtggtttccctcaattgaagacaggaacatcattttccacaggatatgaagagctgccagtaatgccaaaatcttacctcatataatacctggagcatgtgagattcttctagtgaaaaagaacagtcttccctgaagactcagggcttcaacattctagaactgataagtggaccttcagtgtgcaagaatggagaagcatgggatttgcattatgacttgaactgggcttatatctaataatacagagcactatcactaacctcaacagttgacattttaaaagtttttaaatgtatctgaacttgctgttaacacagtgttataactcaagcactagcttcaggaagcatgttgtgttgttaagaagcttttctgatttattctttaacagcatcttgccatctatatgttagtagcagttggcccagaaaggac

SEQ ID NO: 15 - DNA, 4514 bp, Artificial Sequence
  PCSK6 - proprotein convertase subtilisin/kexin type 6
tcgcgggccgaggacgcctctggggcggcaccgcgtcccgagagccccagaagtcggcggggaagtttccccggtggggggcgtttcgggcctcccggacggctctcggccccggagcccggtcgcaggagcgcgggcccgggggcgggaacgcgccgcggccgcctcctcctccccggctcccgcccgcggcggtgttggcggcggcggtggcggcggcggcggcgcttccccggcgcggagcggctttaaaaggcggcactccaccccccggcgcactcgcagctcgggcgccgcgcgagcctgtcgccgctatgcctccgcgcgcgccgcctgcgcccgggccccggccgccgccccgggccgccgccgccaccgacaccgccgcgggcgcggggggcgcggggggcgcggggggcgccggcgggcccgggttccggccgctcgcgccgcgtccctggcgctggctgctgctgctggcgctgcctgccgcctgctccgcgcccccgccgcgccccgtctacaccaaccactgggcggtgcaagtgctgggcggcccggccgaggcggaccgcgtggcggcggcgcacgggtacctcaacttgggccagattggaaacctggaagattactaccatttttatcacagcaaaacctttaaaagatcaaccttgagtagcagaggccctcacaccttcctcagaatggacccccaggtgaaatggctccagcaacaggaagtgaaacgaagggtgaagagacaggtgcgaagtgacccgcaggccctttacttcaacgaccccatttggtccaacatgtggtacctgcattgtggcgacaagaacagtcgctgccggtcggaaatgaatgtccaggcagcgtggaagaggggctacacaggaaaaaacgtggtggtcaccatccttgatgatggcatagagagaaatcaccctgacctggccccaaattatgattcctacgccagctacgacgtgaacggcaatgattatgacccatctccacgatatgatgccagcaatgaaaataaacacggcactcgttgtgcgggagaagttgctgcttcagcaaacaattcctactgcatcgtgggcatagcgtacaatgccaaaataggaggcatccgcatgctggacggcgatgtcacagatgtggtcgaggcaaagtcgctgggcatcagacccaactacatcgacatttacagtgccagctgggggccggacgacgacggcaagacggtggacgggcccggccgactggctaagcaggctttcgagtatggcattaaaaagggccggcagggcctgggctccattttcgtctgggcatctgggaatggcgggagagagggggactactgctcgtgcgatggctacaccaacagcatctacaccatctccgtcagcagcgccaccgagaatggctacaagccctggtacctggaagagtgtgcctccaccctggccaccacctacagcagtggggccttttatgagcgaaaaatcgtcaccacggatctgcgtcagcgctgtaccgatggccacactgggacctcagtctctgcccccatggtggcgggcatcatcgccttggctctagaagcaaacagccagttaacctggagggacgtccagcacctgctagtgaagacatcccggccggcccacctgaaagcgagcgactggaaagtgaacggcgcgggtcataaagttagccatttctatggatttggtttggtggacgcagaagctctcgttgtggaggcaaagaagtggacagcagtgccatcgcagcacatgtgtgtggccgcctcggacaagagacccaggagcatccccttagtgcaggtgctgcggactacggccctgaccagcgcctgcgcggagcactcggaccagcgggtggtctacttggagcacgtggtggttcgcacctccatctcacacccacgccgaggagacctccagatctacctggtttctccctcgggaaccaagtctcaacttctggcaaagaggttgctggatctttccaatgaagggtttacaaactgggaattcatgactgtccactgctggggagaaaaggctgaagggcagtggaccttggaaatccaagatctgccatcccaggtccgcaacccggagaagcaagggaagttgaaagaatggagcctcatactgtatggcacagcagagcacccgtaccacaccttcagtgcccatcagtcccgctcgcggatgctggagctctcagccccagagctggagccacccaaggctgccctgtcaccctcccaggtggaagttcctgaagatgaggaagattacacaggtgtgtgccatccggagtgtggtgacaaaggctgtgatggccccaatgcagaccagtgcttgaactgcgtccacttcagcctggggagtgtcaagaccagcaggaagtgcgtgagtgtgtgccccttgggctactttggggacacagcagcaagacgctgtcgccggtgccacaaggggtgtgagacctgctccagcagagctgcgacgcagtgcctgtcttgccgccgcgggttctatcaccaccaggagatgaacacctgtgtgaccctctgtcctgcaggattttatgctgatgaaagtcagaaaaattgccttaaatgccacccaagctgtaaaaagtgcgtggatgaacctgagaaatgtactgtctgtaaagaaggattcagccttgcacggggcagctgcattcctgactgtgagccaggcacctactttgactcagagctgatcagatgtggggaatgccatcacacctgcggaacctgcgtggggccaggcagagaagagtgcattcactgtgcgaaaaacttccacttccacgactggaagtgtgtgccagcctgtggtgagggcttctacccagaagagatgccgggcttgccccacaaagtgtgtcgaaggtgtgacgagaactgcttgagctgtgcaggctccagcaggaactgtagcaggtgtaagacgggcttcacacagctggggacctcctgcatcaccaaccacacgtgcagcaacgctgacgagacattctgcgagatggtgaagtccaaccggctgtgcgaacggaagctcttcattcagttctgctgccgcacgtgcctcctggccgggtaagggtgcctagctgcccacagagggcaggcactcccatccatccatccgtccaccttcctccagactgtcggccagagtctgtttcaggagcggcgccctgcacctgacagctttatctccccaggagcagcatctctgagcacccaagccaggtgggtggtggctcttaaggaggtgttcctaaaatggtgatatcctctcaaatgctgcttgttggctccagtcttccgacaaactaacaggaacaaaatgaattctgggaatccacagctctggctttggagcagcttctgggaccataagtttactgaatcttcaagaccaaagcagaaaagaaaggcgcttggcatcacacatcactcttctccccgtgcttttctgcggctgtgtagtaaatctccccggcccagctggcgaaccctgggccatcctcacatgtgacaaagggccagcagtctacctgctcgttgcctgccactgagcagtctggggacggtttggtcagactataaataagataggtttgagggcataaaatgtatgaccactggggccggagtatctatttctacatagtcagctacttctgaaactgcagcagtggcttagaaagtccaattccaaagccagaccagaagattctatcccccgcagcgctctcctttgagcaagccgagctctccttgttaccgtgttctgtctgtgtcttcaggagtctcatggcctgaacgaccacctcgacctgatgcagagccttctgaggagaggcaacaggaggcattctgtggccagccaaaaggtaccccgatggccaagcaattcctctgaacaaaatgtaaagccagccatgcattgttaatcatccatcacttcccattttatggaattgcttttaaaatacatttggcctctgcccttcagaagactcgtttttaaggtggaaactcctgtgtctgtgtatattacaagcctacatgacacagttggatttattctgccaaacctgtgtaggcattttataagctacatgttctaatttttaccgatgttaattattttgacaaatatttcatatattttcattgaaatgcacagatctgcttgatcaattcccttgaatagggaagtaacatttgccttaaattttttcgacctcgtctttctccatattgtcctgctcccctgtttgacgacagtgcatttgccttgtcacctgtgagctggagagaacccagatgttgtttattgaatctacaactctgaaagagaaatcaatgaagcaagtacaatgttaaccctaaattaataaaagagttaacatcccatggc

SEQ ID NO: 16 - DNA, 2966 bp, Artificial Sequence
  PTGDR - prostaglandin D2 receptor (DP)
cgcccgagccgcgcgcggagctgccgggggctccttagcacccgggcgccggggccctcgcccttccgcagccttcactccagccctctgctcccgcacgccatgaagtcgccgttctaccgctgccagaacaccacctctgtggaaaaaggcaactcggcggtgatgggcggggtgctcttcagcaccggcctcctgggcaacctgctggccctggggctgctggcgcgctcggggctggggtggtgctcgcggcgtccactgcgcccgctgccctcggtcttctacatgctggtgtgtggcctgacggtcaccgacttgctgggcaagtgcctcctaagcccggtggtgctggctgcctacgctcagaaccggagtctgcgggtgcttgcgcccgcattggacaactcgttgtgccaagccttcgccttcttcatgtccttctttgggctctcctcgacactgcaactcctggccatggcactggagtgctggctctccctagggcaccctttcttctaccgacggcacatcaccctgcgcctgggcgcactggtggccccggtggtgagcgccttctccctggctttctgcgcgctacctttcatgggcttcgggaagttcgtgcagtactgccccggcacctggtgctttatccagatggtccacgaggagggctcgctgtcggtgctggggtactctgtgctctactccagcctcatggcgctgctggtcctcgccaccgtgctgtgcaacctcggcgccatgcgcaacctctatgcgatgcaccggcggctgcagcggcacccgcgctcctgcaccagggactgtgccgagccgcgcgcggacgggagggaagcgtcccctcagcccctggaggagctggatcacctcctgctgctggcgctgatgaccgtgctcttcactatgtgttctctgcccgtaatttatcgcgcttactatggagcatttaaggatgtcaaggagaaaaacaggacctctgaagaagcagaagacctccgagccttgcgatttctatctgtgatttcaattgtggacccttggatttttatcattttcagatctccagtatttcggatattttttcacaagattttcattagacctcttaggtacaggagccggtgcagcaattccactaacatggaatccagtctgtgacagtgtttttcactctgtggtaagctgaggaatatgtcacattttcagtcaaagaaccatgattaaaaaaaaaaagacaacttacaatttaaatccttaaaagttacctcccataacaaaagcatgtatatgtattttcaaaagtatttgatatcttaacaatgtgttaccattctatagtcatgaaccccttcagtgcattttcatttttttattaacagcaactaaaattttatatattgtaaccagtgttaaaagtcttaaaaaacaatggtattaattgtccctacatttgtgcttggtggccctattttttttttttagagaggccttgagacatacaggtcttttaaaatacagtagaaacaccactgtttacgattatacgatggacattcataaaaagcataatttcttaccctattcattttttggtgaaacctgattcattgattttatatcattgccgatgtttagttcatttctttgccaattgatctaagcatagcctgaattatgatgttcctcagagaagtgaggtgggaaatatgaccaggtcaggcagttggaggggcttccccagccaccatcggggagtacttgctgcctcaggtggagacctgaagctgtaactagatgcagagcaagatatgactatagcccacaacccaaagaagcaaaaattcgtttttatcttttgaaatccagtttcttttgtattgagtcaagggtgtcagtaggaatcaaaagttgggggtgggttgcaaaatgttctttcagtttttagaacctccattttataaaagaattatcctatcaatggattctttagtggaaggatttatgcttctttgaaaaccagtgtgtgactcactgtagagccatgtttactgtttgactgtgtggcacaggggggcatttggcacagcaaaaagcccacccaggacttagcctcagttgacgatagtaacaatggccttaacatctaccttaacagctacctattacagccgtattctgctgtccgtggagacggtaagatcttaggttccaagattttacttcaaattacaccttcaaaactggagcagcatatagccgaaaaggagcacaactgagcactttaatagtaatttaaaagttttcaagggtcagcaatatgatgactgaaagggaaaagtggaggaaacgcagctgcaactgaagcggagactctaaacccagcttgcaggtaagagctttcacctttggtaaaagaacagctggggaggttcaaggggtttcagcatctctggagttcctttgtatctgacaatctcaggactccaaggtgcaaagcctgctgcatttgcgtgatctcaagacctccagccagaagtcccttccaaatataagagtactcatgtttatttatttccaactgagcagcaacctcctttgtttcacttatgttttttccagtatctgagataatataaagctgggtaattttttatgtaattttttggtatagcaaaactgtgaaaaagccaaattaggcatacaaggagtatgatttaacagtatgacatgatgaaaaaaatacagttgtttttgaaatttaacttttgtttgtaccttcaatgtgtaagtacatgcatgttttattgtcagaggaagaacatgttttttgtattctttttttggagaggtgtgttaggataattgtccagttaatttgaaaaggccccagatgaatcaataaatataattttatagtaaaaaaaaaaaaaaaaaaaaaaaaa

SEQ ID NO: 17 - protein, 1446 aa, Artificial Sequence
  PTCH - patched homolog of Drosophila
MASAGNAAEPQDRGGGGSGCIGAPGRPAGGGRRRRTGGLRRAAAPDRDYLHRPSYCDAAFALEQISKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPLYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 18 - protein, 908 aa, Artificial Sequence
  PSMD2 - proteasome (prosome, macropain) 26S subunit, non-ATPase 2
MEEGGRDKAPVQPQQSPAAAPGGTDEKPSGKERRDAGDKDKEQELSEEDKQLQDELEMLVERLGEKDTSLYRPALEELRRQIRSSTTSMTSVPKPLKFLRPHYGKLKEIYENMAPGENKRFAADIISVLAMTMSGERECLKYRLVGSQEELASWGHEYVRHLAGEVAKEWQELDDAEKVQREPLLTLVKEIVPYNMAHNAEHEACDLLMEIEQVDMLEKDIDENAYAKVCLYLTSCVNYVPEPENSALLRCALGVFRKFSRFPEALRLALMLNDMELVEDIFTSCKDVVVQKQMAFMLGRHGVFLELSEDVEEYEDLTEIMSNVQLNSNFLALARELDIMEPKVPDDIYKTHLENNRFGGSGSQVDSARMNLASSFVNGFVNAAFGQDKLLTDDGNKWLYKNKDHGMLSAAASLGMILLWDVDGGLTQIDKYLYSSEDYIKSGALLACGIVNSGVRNECDPALALLSDYVLHNSNTMRLGSIFGLGLAYAGSNREDVLTLLLPVMGDSKSSMEVAGVTALACGMIAVGSCNGDVTSTILQTIMEKSETELKDTYARWLPLGLGLNHLGKGEAIEAILAALEVVSEPFRSFANTLVDVCAYAGSGNVLKVQQLLHICSEHFDSKEKEEDKDKKEKKDKDKKEAPADMGAHQGVAVLGIALIAMGEEIGAEMALRTFGHLLRYGEPTLRRAVPLALALISVSNPRLNILDTLSKFSHDADPEVSYNSIFAMGMVGSGTNNARLAAMLRQLAQYHAKDPNNLFMVRLAQGLTHLGKGTLTLCPYHSDRQLMSQVAVAGLLTVLVSFLDVRNIILGKSHYVLYGLVAAMQPRMLVTFDEELRPLPVSVRVGQAVDVVGQAGKPKTITGFQTHTTPVLLAHGERAELATEEFLPVTPILEGFVILRKNPNYDL

SEQ ID NO: 19 - protein, 496 aa, Artificial Sequence
  NMT 1 - N-myristoyltransferase 1
MADESETAVKPPAPPLPQMMEGNGNGHEHCSDCENEEDNSYNRGGLSPANDTGAKKKKKKQKKKKEKGSETDSAQDQPVKMNSLPAERIQEIQKAIELFSVGQGPAKTMEEASKRSYQFWDTQPVPKLGEVVNTHGPVEPDKDNIRQEPYTLPQGFTWDALDLGDRGVLKELYTLLNENYVEDDDNMFRFDYSPEFLLWALRPPGWLPQWHCGVRVVSSRKLVGFISAIPANIHIYDTEKKMVEINFLCVHKKLRSKRVAPVLIREITRRVHLEGIFQAVYTAGVVLPKPVGTCRYWHRSLNPRKLIEVKFSHLSRNMTMQRTMKLYRLPETPKTAGLRPMETKDIPVVHQLLTRYLKQFHLTPVMSQEEVEHWFYPQENIIDTFVVENANGEVTDFLSFYTLPSTIMNHPTHKSLKAAYSFYNVHTQTPLLDLMSDALVLAKMKGFDVFNALDLMENKTFLEKLKFGIGDGNLQYYLYNWKCPSMGAEKVGLVLQ

SEQ ID NO: 20 - protein, 520 aa, Artificial Sequence
  MARCO - macrophage receptor with collagenous structure
MRNKKILKEDELLSETQQAAFHQIAMEPFEINVPKPKRRNGVNFSLAVVVIYLILLTAGAGLLVVQVLNLQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQGQPGLQGVPGPPGAVGHPGAKGEPGSAGSPGRAGLPGSPGSPGATGLKGSKGDTGLQGQQGRKGESGVPGPAGVKGEQGSPGLAGPKGAPGQAGQKGDQGVKGSSGEQGVKGEKGERGENSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 21 - protein, 326 aa, Artificial Sequence
  CDK6 - cyclin-dependent kinase
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 22 - protein, 438 aa, Artificial Sequence
  FLJ16046 - MDCK gene (Madin Darby Canine Kidney)
MMYAPVEFSEAEFSRAEYQRKQQFWDSVRLALFTLAIVAIIGIAIGIVTHFVVEDDKSFYYLASFKVTNIKYKENYGIRSSREFIERSHQIERMMSRIFRHSSVGGRFIKSHVIKLSPDEQGVDILIVLIFRYPSTDSAEQIKKKIEKALYQSLKTKQLSLTLNKPSFRLTPIDSKKMRNLLNSRCGIRMTSSNMPLPASSSTQRIVQGRETAMEGEWPWQASLQLIGSGHQCGASLISNTWLLTAAHCFWKNKDPTQWIATFGATITPPAVKRNVRKIILHENYHRETNENDIALVQLSTGVEFSNIVQRVCLPDSSIKLPPKTSVFVTGFGSIVDDGPIQNTLRQARVETISTDVCNRKDVYDGLITPGMLCAGFMEGKIDACKGDSGGPLVYDNHDIWYIVGIVSWGQSCALPKKPGVYTRVTKYRDWIASKTGM

SEQ ID NO: 23 - protein, 956 aa, Artificial Sequence
  PCSK6 - proprotein convertase subtilisin/kexin type 6
MPPRAPPAPGPRPPPRAAAATDTAAGAGGAGGAGGAGGPGFRPLAPRPWRWLLLLALPAACSAPPPRPVYTNHWAVQVLGGPAEADRVAAAHGYLNLGQIGNLEDYYHFYHSKTFKRSTLSSRGPHTFLRMDPQVKWLQQQEVKRRVKRQVRSDPQALYFNDPIWSNMWYLHCGDKNSRCRSEMNVQAAWKRGYTGKNVVVTILDDGIERNHPDLAPNYDSYASYDVNGNDYDPSPRYDASNENKHGTRCAGEVAASANNSYCIVGIAYNAKIGGIRMLDGDVTDVVEAKSLGIRPNYIDIYSASWGPDDDGKTVDGPGRLAKQAFEYGIKKGRQGLGSIFVWASGNGGREGDYCSCDGYTNSIYTISVSSATENGYKPWYLEECASTLATTYSSGAFYERKIVTTDLRQRCTDGHTGTSVSAPMVAGIIALALEANSQLTWRDVQHLLVKTSRPAHLKASDWKVNGAGHKVSHFYGFGLVDAEALVVEAKKWTAVPSQHMCVAASDKRPRSIPLVQVLRTTALTSACAEHSDQRVVYLEHVVVRTSISHPRRGDLQIYLVSPSGTKSQLLAKRLLDLSNEGFTNWEFMTVHCWGEKAEGQWTLEIQDLPSQVRNPEKQGKLKEWSLILYGTAEHPYHTFSAHQSRSRMLELSAPELEPPKAALSPSQVEVPEDEEDYTGVCHPECGDKGCDGPNADQCLNCVHFSLGSVKTSRKCVSVCPLGYFGDTAARRCRRCHKGCETCSSRAATQCLSCRRGFYHHQEMNTCVTLCPAGFYADESQKNCLKCHPSCKKCVDEPEKCTVCKEGFSLARGSCIPDCEPGTYFDSELIRCGECHHTCGTCVGPGREECIHCAKNFHFHDWKCVPACGEGFYPEEMPGLPHKVCRRCDENCLSCAGSSRNCSRCKTGFTQLGTSCITNHTCSNADETFCEMVKSNRLCERKLFIQFCCRTCLLAG

SEQ ID NO: 24 - protein, 359 aa, Artificial Sequence
  PTGDR - prostaglandin D2 receptor (DP)
MKSPFYRCQNTTSVEKGNSAVMGGVLFSTGLLGNLLALGLLARSGLGWCSRRPLRPLPSVFYMLVCGLTVTDLLGKCLLSPVVLAAYAQNRSLRVLAPALDNSLCQAFAFFMSFFGLSSTLQLLAMALECWLSLGHPFFYRRHITLRLGALVAPVVSAFSLAFCALPFMGFGKFVQYCPGTWCFIQMVHEEGSLSVLGYSVLYSSLMALLVLATVLCNLGAMRNLYAMHRRLQRHPRSCTRDCAEPRADGREASPQPLEELDHLLLLALMTVLFTMCSLPVIYRAYYGAFKDVKEKNRTSEEAEDLRALRFLSVISIVDPWIFIIFRSPVFRIFFHKIFIRPLRYRSRCSNSTNMESSL

(19) United States
(12) Patent Application Publication
(10) Pub. No.: US 2009/0285819 A1
(43) Pub. Date: Nov. 19, 2009

(54) METHODS AND COMPOSITIONS FOR TREATING INFLUENZA

(75) Inventors: Limin Li, Bethesda, MD (US); Michael Kinch, Laytonsville, MD (US); Michael Goldblatt, McLean, VA (US)

Correspondence Address: Steven B. Kelber;Berenato, White & Stavish, 6550 Rock Spring Drive, Suite 240, Bethesda, MD, 20817, US

(73) Assignee: Functional Genetics, Inc., Gaithersburg, MD (US)

(21) Appl. No.: 11/940,418

(22) Filed: Nov. 15, 2007

Related U.S. Application Data
(60) Provisional application No. 60/858,920, filed on Nov. 15, 2006.

(51) Int. Cl. A61K 39/395; A61K 31/7088; A61K 39/00; C07K 16/18

(52) U.S. Cl. 424/139.1; 424/130.1; 424/184.1; 530/387.9

(57) ABSTRACT

Genes relating to resistance to infection by influenza virus are identified. The genes and the gene products (i.e., the polynucleotides transcribed from and polypeptides encoded by the genes) can be used for the prevention and treatment of influenza. The genes and the gene products can also be used to screen agents that modulate the gene expression or the activities of the gene products.

### CROSS-REFERENCE TO RELATED APPLICATIONS

This application claims the benefit, under 35 U.S.C. § 119(e), of U.S. Provisional Patent Application No. 60/858,920, filed on Nov. 15, 2006, which is hereby incorporated by reference in its entirety.

The present invention relates generally to the treatment of viral diseases, and in particular to diseases caused by influenza virus. The invention also relates to influenza resistant genes, polynucleotides transcribed from these genes and polypeptides encoded by these genes.

### BACKGROUND OF THE INVENTION

Influenza, also known as the flu, is a contagious disease that is caused by the influenza virus. It attacks the respiratory tract in humans (nose, throat, and lungs). There are three types of influenza viruses, influenza A, B and C. Influenza A can infect humans and other animals while influenza B and C infect only humans.

Most people who get influenza will recover in one to two weeks, but some people will develop life-threatening complications (such as pneumonia) as a result of the flu. Millions of people in the United States—about 5% to 20% of U.S. residents—will get influenza each year. An average of about 36,000 people per year in the United States die from influenza, and 114,000 per year have to be admitted to the hospital as a result of influenza. People age 65 years and older, people of any age with chronic medical conditions, and very young children are more likely to get complications from influenza. Pneumonia, bronchitis, and sinus and ear infections are three examples of complications from flu. The flu can also make chronic health problems worse. For example, people with asthma may experience asthma attacks while they have the flu, and people with chronic congestive heart failure may have worsening of this condition that is triggered by the flu.

Vaccination is the primary method for preventing influenza and its severe complications. Studies revealed that vaccination is associated with reductions in influenza-related respiratory illness and physician visits among all age groups, hospitalization and death among persons at high risk, otitis media among children, and work absenteeism among adults (18). The major problem with vaccination is that new vaccine has to be prepared for each flu season and the vaccine production is a tedious and costly process.

Although influenza vaccination remains the cornerstone for the control and treatment of influenza, three antiviral drugs (amantadine, rimantadine, and oseltamivir) have been approved for preventing and treating flu. When used for prevention, they are about 70% to 90% effective for preventing illness in healthy adults. When used for treating flu, these drugs can reduce the symptoms of the flu and shorten the time you are sick by 1 or 2 days. They also can make you less contagious to others. However, the treatment must begin within 2 days of the onset of symptoms for it to be effective. There is a need in the art for improved methods for treating influenza.

### SUMMARY OF THE INVENTION

One aspect of the present invention relates to influenza resistant genes (IRGs) and the gene products (IRG products), which include the polynucleotides transcribed from the IRGs (IRGPNs) and the polypeptides encoded by the IRGs (IRGPPs).

In one embodiment, the present invention provides pharmaceutical compositions for the treatment of influenza. The pharmaceutical compositions comprise a pharmaceutically acceptable carrier and at least one of the following: (1) an IRG product; (2) an agent that modulates an activity of an IRG product; and (3) an agent that modulates the expression of an IRG.

In another embodiment, the present invention provides methods for treating influenza in a patient with the pharmaceutical compositions described above. The patient may be afflicted with influenza, in which case the methods provide treatment for the disease. The patient may also be considered at risk for influenza, in which case the methods provide prevention for disease development.

In another embodiment, the present invention provides methods for screening anti-influenza agents based on the agents' interaction with IRGPPs, or the agents' effect on the activity or expression of IRGPPs.

In another embodiment, the present invention provides biochips for screening anti-influenza agents. The biochips comprise at least one of the following (1) an IRGPP or its variant, (2) a portion of an IRGPP or its variant (3) an IRGPN or its variant, and (4) a portion of an IRGPN or its variant.

### BRIEF DESCRIPTION OF FIGURES

FIG. 1 depicts the process for screening influenza resistant clones.

FIG. 2A is the alignment of the 5′-end flanking sequences obtained from three subclones of influenza resistant clone 26-8-7; FIG. 2B depicts the genomic site of the RHKO integration; and FIG. 2C is a schematic map of integration.

FIG. 3A is the alignment of the 5′-end flanking sequences obtained from two subclones of influenza resistant clone R18-6; FIG. 3B depicts the genomic site of the RHKO integration; and FIG. 3C is a schematic map of integration.

FIG. 4A is the alignment of the 5′-end flanking sequences obtained from three subclones of influenza resistant clone 26-8-11; FIG. 4B depicts the genomic site of the RHKO integration; and FIG. 4C is a schematic map of integration.

FIG. 5A is the alignment of the 5′-end flanking sequences obtained from three subclones of influenza resistant clone R15-6; FIG. 5B depicts the genomic site of the RHKO integration; and FIG. 5C is a schematic map of integration.

FIG. 6A is the alignment of the 5′-end flanking sequences obtained from three subclones of influenza resistant clone R21-1; FIG. 6B depicts the genomic site of the RHKO integration; and FIG. 6C is a schematic map of integration.

FIG. 7 depicts the genomic site of the RHKO integration in influenza resistant clone R27-32.

FIG. 8A is the alignment of the 5′-end flanking sequences obtained from two subclones of influenza resistant clone R27-3-33; FIG. 8B depicts the genomic site of the RHKO integration; and FIG. 8C is a schematic map of integration.

FIG. 9A depicts the genomic site of RHKO integration in influenza resistant clone R27-3-35 and FIG. 9B is a schematic map of integration.

### DETAILED DESCRIPTION OF THE INVENTION

The preferred embodiments of the invention are described below. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase.

It is further intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

Further examples exist throughout the disclosure, and it is not applicant's intention to exclude from the scope of his invention the use of structures, materials, methods, or acts that are not expressly identified in the specification, but nonetheless are capable of performing a claimed function.

The present invention is generally directed to compositions and methods for the treatment and prevention of influenza; and to the identification of novel therapeutic agents for influenza. The present invention is based on the finding that modulation of certain gene expression leads to resistance to the infection by influenza virus.

### DEFINITIONS AND TERMS

To facilitate an understanding of the present invention, a number of terms and phrases are defined below:

As used herein, the term “influenza resistant gene (IRG)” refer to a gene whose inhibition or over-expression leads to resistance to infection by influenza virus. IRGs generally refer to the genes listed in Table 3.

As used herein, the terms “IRG-related polynucleotide”, “IRG-polynucleotide” and “IRGPN” are used interchangeably. The terms include a transcribed polynucleotide (e.g., DNA, cDNA or mRNA) that comprises one of the IRG sequences or a portion thereof.

As used herein, the terms “IRG-related polypeptide (IRGPP)”, “IRG protein” and “IRGPP” are used interchangeably. The terms include polypeptides encoded by an IRG, an IRGPN, or a portion of an IRG or IRGPN.

As used herein, an “IRG product” includes a nucleic acid sequence and an amino acid sequence (e.g., a polynucleotide or polypeptide) generated when an IRG is transcribed and/or translated. Specifically, IRG products include IRGPNs and IRGPPs.

As used herein, a “variant of a polynucleotide” includes a polynucleotide that differs from the original polynucleotide by one or more substitutions, additions, deletions and/or insertions such that the activity of the encoded polypeptide is not substantially changed (e.g., the activity may be diminished or enhanced, by less than 50%, and preferably less than 20%) relative to the polypeptide encoded by the original polynucleotide.

A variant of a polynucleotide also includes polynucleotides that are capable of hybridizing under reduced stringency conditions, more preferably stringent conditions, and most preferably highly stringent conditions to the original polynucleotide (or a complementary sequence). Examples of conditions of different stringency are listed in Table 2.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention.

As used herein, a “variant of a polypeptide” is a polypeptide that differs from a native polypeptide in one or more substitutions, deletions, additions and/or insertions, such that the bioactivity or immunogenicity of the native polypeptide is not substantially diminished. In other words, the bioactivity of a variant polypeptide or the ability of a variant polypeptide to react with antigen-specific antisera may be enhanced or diminished by less than 50%, and preferably less than 20%, relative to the native polypeptide. Variant polypeptides include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Modifications and changes can be made in the structure of a polypeptide of the present invention and still obtain a molecule having biological activity and/or immunogenic properties. Because it is the interactive capacity and nature of a polypeptide that defines that polypeptide's biological activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art. It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those that are within +/−1 are particularly preferred, and those within +/−0.5 are even more particularly preferred.

Substitution of like amino acids can also be made on the basis of hydrophilicity, particularly where the biological functional equivalent polypeptide or polypeptide fragment, is intended for use in immunological embodiments. U.S. Pat. No. 4,554,101, incorporated hereinafter by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of an IRGPP as set forth above.’

TABLE 1
Amino Acid Substitutions

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure, tertiary structure, and hydropathic nature of the polypeptide.

Polypeptide variants preferably exhibit at least about 70%, more preferably at least about 90% and most preferably at least about 95% sequence homology to the original polypeptide.

A polypeptide variant also includes a polypeptide that is modified from the original polypeptide by either natural processes, such as post-translational processing, or by chemical modification techniques which are well known in the art. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a fluorophore or a chromophore, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

As used herein, a “biologically active portion” of an IRGPP includes a fragment of an IRGPP comprising amino acid sequences sufficiently homologous to or derived from the amino acid sequence of the IRGPP, which includes fewer amino acids than the full length IRGPP, and exhibits at least one activity of the IRGPP. Typically, a biologically active portion of an IRGPP comprises a domain or motif with at least one activity of the IRGPP. A biologically active portion of an IRGPP can be a polypeptide which is, for example, 10, 25, 50, 100, 200 or more amino acids in length. Biologically active portions of an IRGPP can be used as targets for developing agents which modulate an IRGPP-mediated activity.

As used herein, an “immunogenic portion,” an “antigen,” an “immunogen,” or an “epitope” of an IRGPP includes a fragment of an IRGPP comprising an amino acid sequence sufficiently homologous to, or derived from, the amino acid sequence of the IRGPP, which includes fewer amino acids than the full length IRGPP and can be used to induce an anti-IRGPP humoral and/or cellular immune response.

As used herein, the term “modulation” includes, in its various grammatical forms (e.g., “modulated”, “modulation”, “modulating”, etc.), up-regulation, induction, stimulation, potentiation, and/or relief of inhibition, as well as inhibition and/or down-regulation or suppression.

As used herein, the term “control sequences” or “regulatory sequences” refers to DNA sequences necessary for the expression of an operably linked coding sequence in a particular host organism. The term “control/regulatory sequence” is intended to include promoters, enhancers and other expression control elements (e.g., polyadenylation signals). Control/regulatory sequences include those which direct constitutive expression of a nucleotide sequence in many types of host cells and those which direct expression of the nucleotide sequence only in certain host cells (e.g., tissue-specific regulatory sequences).

A nucleic acid sequence is “operably linked” to another nucleic acid sequence when the former is placed into a functional relationship with the latter. For example, a DNA for a presequence or secretory leader peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice.

As used herein, the “stringency” of a hybridization reaction refers to the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table 2 below: highly stringent conditions are those that are at least as stringent as conditions A-F; stringent conditions are at least as stringent as conditions G-L; and reduced stringency conditions are at least as stringent as conditions M-R.

TABLE 2
Stringency Conditions

| | Poly- | | | Wash |
|---|---|---|---|---|
| Stringency | nucleotide | Hybrid | Hybridization | Temperature |
| Condition | Hybrid | Length (bp)¹ | Temperature and Buffer^{H} | and Buffer^{H} |
| A | DNA:DNA | >50 | 65° C.; 1xSSC -or- | 65° C.; |
| | | | 42° C.; 1xSSC, 50% formamide | 0.3xSSC |
| B | DNA:DNA | <50 | T_{B}*; 1xSSC | T_{B}*; 1xSSC |
| C | DNA:RNA | >50 | 67° C.; 1xSSC -or- | 67° C.; |
| | | | 45° C.; 1xSSC, 50% formamide | 0.3xSSC |
| D | DNA:RNA | <50 | T_{D}*; 1xSSC | T_{D}*; 1xSSC |
| E | RNA:RNA | >50 | 70° C.; 1xSSC -or- | 70° C.; |
| | | | 50° C.; 1xSSC, 50% formamide | 0.3xSSC |
| F | RNA:RNA | <50 | T_{F}*; 1xSSC | T_{F}*; 1xSSC |
| G | DNA:DNA | >50 | 65° C.; 4xSSC -or- | 65° C.; |
| | | | 42° C.; 4xSSC, 50% formamide | 1xSSC |
| H | DNA:DNA | <50 | T_{H}*; 4xSSC | T_{H}*; 4xSSC |
| I | DNA:RNA | >50 | 67° C.; 4xSSC -or- | 67° C.; |
| | | | 45° C.; 4xSSC, 50% formamide | 1xSSC |
| J | DNA:RNA | <50 | T_{J}*; 4xSSC | T_{J}*; 4xSSC |
| K | RNA:RNA | >50 | 70° C.; 4xSSC -or- | 67° C.; |
| | | | 50° C.; 4xSSC, 50% formamide | 1xSSC |
| L | RNA:RNA | <50 | T_{L}*; 2xSSC | T_{L}*; 2xSSC |
| M | DNA:DNA | >50 | 50° C.; 4xSSC -or- | 50° C.; |
| | | | 40° C.; 6xSSC, 50% formamide | 2xSSC |
| N | DNA:DNA | <50 | T_{N}*; 6xSSC | T_{N}*; 6xSSC |
| O | DNA:RNA | >50 | 55° C.; 4xSSC -or- | 55° C.; |
| | | | 42° C.; 6xSSC, 50% formamide | 2xSSC |
| P | DNA:RNA | <50 | T_{P}*; 6xSSC | T_{P}*; 6xSSC |
| Q | RNA:RNA | >50 | 60° C.; 4xSSC -or- | 60° C.; |
| | | | 45° C.; 6xSSC, 50% formamide | 2xSSC |
| R | RNA:RNA | <50 | T_{R}*; 4xSSC | T_{R}*; 4xSSC |

¹ The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H} SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}* The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5⁺ 16.6(log₁₀Na⁺)⁺0.41(% G⁺ C) − (600/N), where N is the number of bases in the hybrid, and Na⁺ is the concentration of sodium ions in the hybridization buffer (Na⁺ for 1xSSC = 0.165M).

As used herein, the terms “immunospecific binding” and “specifically bind to” refer to antibodies that bind to an antigen with a binding affinity of 10⁵ M⁻¹.

As used herein, the terms “treating,” “treatment,” and “therapy” refer to curative therapy, prophylactic therapy, and preventative therapy.

Various aspects of the invention are described in further detail in the following subsections. The subsections below describe in more detail the present invention. The use of subsections is not meant to limit the invention; subsections may apply to any aspect of the invention.

### Influenza Resistant Genes (IRGs)

One aspect of the present invention relates to influenza resistance genes (IRGs). Briefly, Madin Darby Canine Kidney (MDCK) cells were infected with a retro-viral based random homozygous knock-out (RHKO) vector. Cells containing the stably integrated vector were selected and subjected to influenza infection using the MOI which would result in 100% killing of parental cells between 48 to 72 hour. The influenza resistant cells were expanded and subject to additional rounds of influenza infection with higher multiplicity of infection (MOI). The resistant clones that survived multiple rounds of influenza infection were recovered. The influenza resistant phenotype was validated by testing the clones' resistance to multiple strains of influenza virus and by correlation of the phenotype with RHKO integration. The RHKO integration sites in the resistant cells were then cloned and identified. The affected genes are identified by aligning the flanking sequences at the integration site to the Genbank database. It should be noted that the affected genes, which are referred to as influenza resistant genes hereinafter, are either under-expressed (i.e., inhibited by RHKO integration) or over-expressed (i.e., enhanced by RHKO integration) in the influenza resistant cells.

Table 3 provides a list of the genes that, when over-expressed or under-expressed in a cell, lead to resistance to influenza virus infection. Accordingly, genes listed in Table 3 are designated as influenza resistance genes (IRGs).

| | | 5′-flanking | | | |
|---|---|---|---|---|---|
| | | seq at | | | predicted |
| | Locus | insertion | cDNA | Amino acid | effect of |
| Gene | ID | site | sequence | sequence | integration |
| PTCH | 5727 | SEQ ID | SEQ ID | SEQ ID | antisense |
| | | NO: 1 | NO: 9 | NO: 17 | |
| PSMD2 | 5708 | SEQ ID | SEQ ID | SEQ ID | over- |
| | | NO: 2 | NO: 10 | NO: 18 | expression |
| NMT 1 | 4836 | SEQ ID | SEQ ID | SEQ ID | over- |
| | | NO: 3 | NO: 11 | NO: 19 | expression |
| MARCO | 8685 | SEQ ID | SEQ ID | SEQ ID | disruption of |
| | | NO: 4 | NO: 12 | NO: 20 | promoter |
| CDK6 | 1021 | SEQ ID | SEQ ID | SEQ ID | disruption of |
| | | NO: 5 | NO: 13 | NO: 21 | promoter |
| FLJ16046 | 389208 | SEQ ID | SEQ ID | SEQ ID | over- |
| | | NO: 6 | NO: 14 | NO: 22 | expression |
| PCSK6 | 5046 | SEQ ID | SEQ ID | SEQ ID | antisense |
| | | NO: 7 | NO: 15 | NO: 23 | |
| PTGDR | 5729 | SEQ ID | SEQ ID | SEQ ID | antisense |
| | | NO: 8 | NO: 16 | NO: 24 | |

Briefly, PTCH (patched homolog of Drosophila) encodes a member of the patched gene family. The encoded protein is the receptor for sonic hedgehog, a secreted molecule implicated in the formation of embryonic structures and in tumorigenesis. This gene functions as a tumor suppressor. Mutations of this gene have been associated with nevoid basal cell carcinoma syndrome, esophageal squamous cell carcinoma, trichoepitheliomas, transitional cell carcinomas of the bladder, as well as holoprosencephaly. Alternative spliced variants have been described, but their full length sequences have not be determined.

PSMD2 (proteasome (prosome, macropain) 26S subunit, non-ATPase 2) encodes a multicatalytic proteinase complex with a highly ordered structure composed of 2 complexes, a 20S core and a 19S regulator. The 20S core is composed of 4 rings of 28 non-identical subunits; 2 rings are composed of 7 alpha subunits and 2 rings are composed of 7 beta subunits. The 19S regulator is composed of a base, which contains 6 ATPase subunits and 2 non-ATPase subunits, and a lid, which contains up to 10 non-ATPase subunits. Proteasomes are distributed throughout eukaryotic cells at a high concentration and cleave peptides in an ATP/ubiquitin-dependent process in a non-lysosomal pathway. An essential function of a modified proteasome, the immunoproteasome, is the processing of class I MHC peptides. This gene encodes one of the non-ATPase subunits of the 19S regulator lid. In addition to participation in proteasome function, this subunit may also participate in the TNF signalling pathway since it interacts with the tumor necrosis factor type 1 receptor. A pseudogene has been identified on chromosome 1.

NMT1 (N-myristoyltransferase 1) encodes N-Myristoyltransferase which is an essential eukaryotic enzyme that catalyzes the cotranslational and/or posttranslational transfer of myristate to the amino terminal glycine residue of a number of important proteins especially the non-receptor tyrosine kinases whose activity is important for tumorigenesis. Human NMT was found to be phosphorylated by non-receptor tyrosine kinase family members of Lyn, Fyn and Lck and dephosphorylated by the Ca(2+)/calmodulin-dependent protein phosphatase, calcineurin. NMT has been associated with HIV particle formation and budding. Chronically HIV-1-infected T-cell line CEM/LAV-1 exhibited low expression levels of NMT (Takamune et al., FEBS Lett. 506:81-84, 2001).

MARCO (macrophage receptor with collagenous structure) encodes a member of the class A scavenger receptor family which is part of the innate antimicrobial immune system. The protein may bind both Gram-negative and Gram-positive bacteria via an extracellular, C-terminal, scavenger receptor cysteine-rich (SRCR) domain. In addition to short cytoplasmic and transmembrane domains, there is an extracellular spacer domain and a long, extracellular collagenous domain. The protein may form a trimeric molecule by the association of the collagenous domains of three identical polypeptide chains.

CDK6 (cyclin-dependent kinase) encodes a member of the cyclin-dependent protein kinase (CDK) family. CDK family members are highly similar to the gene products of Saccharomyces cerevisiae cdc28, and Schizosaccharomyces pombe cdc2, and are known to be important regulators of cell cycle progression. This kinase is a catalytic subunit of the protein kinase complex that is important for cell cycle G1 phase progression and G1/S transition. The activity of this kinase first appears in mid-G1 phase, which is controlled by the regulatory subunits including D-type cyclins and members of INK4 family of CDK inhibitors. This kinase, as well as CDK4, has been shown to phosphorylate, and thus regulate the activity of, tumor suppressor protein Rb.

FLJ16046 encodes the last exon of a novel protein. The protein share some homology with a domain found in sea urchin sperm protein, enterokinase, and the trans membrane domain of tyrosine-like serine protease.

PCSK6 (proprotein convertase subtilisin/kexin type 6) encodes a protein of the subtilisin-like proprotein convertase family. The members of this family are proprotein convertases that process latent precursor proteins into their biologically active products. This encoded protein is a calcium-dependent serine endoprotease that can cleave precursor protein at their paired basic amino acid processing sites. Some of its substrates are—transforming growth factor beta related proteins, proalbumin, and von Willebrand factor. This gene is thought to play a role in tumor progression. There are eight alternatively spliced transcript variants encoding different isoforms described for this gene.

PTGDR (prostaglandin D2 receptor (DP)) encodes a G-protein-coupled receptor that has been shown to function as a prostanoid DP receptor. The activity of this receptor is mainly mediated by G-S proteins that stimulate adenylate cyclase resulting in an elevation of intracellular cAMP and Ca²⁺. Knockout studies in mice suggest that the ligand of this receptor, prostaglandin D2 (PGD2), functions as a mast cell-derived mediator to trigger asthmatic responses.

### IRGs and IRG Products as Therapeutic Targets for Influenza

In general, Table 3 provides genes that relate to a cell's susceptibility to influenza virus infection. The IRGs of Table 3, as well as the corresponding IRG products (IRGPN and IRGPP) may become novel therapeutic targets for the treatment and prevention of influenza. The IRGs can be used to produce antibodies specific to IRG products, and to construct gene therapy vectors that inhibit the development of influenza. In addition, the IRG products themselves may be used as therapeutic agent for influenza.

The IRGs listed in Table 3 can be administered for gene therapy purposes, including the administration of antisense nucleic acids and RNAi. The IRG products (including IRGPPs and IRGPNs) and modulator of IRG products (such as anti-IRGPP antibodies) can also be administered as therapeutic drugs.

For example, the inhibition of IRG PTCH expression leads to resistance to influenza virus infection. Accordingly, influenza may be prevented or treated by down-regulating the PTCH expression. Similarly, the over-expression of IRG NMT1 leads to resistance to influenza virus infection. Accordingly, influenza may be prevented or treated by enhancing NMT1 expression.

### Sources of IRG Products

The IRG products (IRGPNs and IRGPPs) of the invention may be isolated from any tissue or cell of a subject. It will be apparent to one skilled in the art that bodily fluids, such as blood, may also serve as sources from which the IRG product of the invention may be assessed. A biological sample may comprise biological components such as blood plasma, serum, erythrocytes, leukocytes, blood platelets, lymphocytes, macrophages, fibroblast cells, mast cells, fat cells, neuronal cells, epithelial cells and the like. The tissue samples containing one or more of the IRG product themselves may be useful in the methods of the invention, and one skilled in the art will be cognizant of the methods by which such samples may be conveniently obtained, stored and/or preserved.

### Isolated Polynucleotides

One aspect of the invention pertains to isolated polynucleotides. Another aspect of the invention pertains to isolated polynucleotide fragments sufficient for use as hybridization probes to identify an IRGPN in a sample, as well as nucleotide fragments for use as PCR probes/primers of the amplification or mutation of the nucleic acid molecules which encode the IRGPP of the invention.

An IRGPN molecule of the present invention, e.g., a polynucleotide molecule having the nucleotide sequence of one of the IRGs listed in Table 3, or homologs thereof, or a portion thereof, can be isolated using standard molecular biology techniques and the sequence information provided herein, as well as sequence information known in the art. Using all or a portion of the polynucleotide sequence of one of the IRGs listed Table 3 (or a homolog thereof) as a hybridization probe, an IRG of the invention or an IRGPN of the invention can be isolated using standard hybridization and cloning techniques.

An IRGPN of the invention can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to IRG nucleotide sequences of the invention can be prepared by standard synthetic techniques, e.g., using an automated DNA synthesizer.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. One such amplification technique is inverse PCR, which uses restriction enzymes to generate a fragment in the known region of the gene. A variation on this procedure, which employs two primers that initiate extension in opposite directions from the known sequence, is described in WO 96/38591.

Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:11-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In another preferred embodiment, an isolated polynucleotide molecule of the invention comprises a polynucleotide molecule which is a complement of the nucleotide sequence of an IRG listed in Table 3, or homolog thereof, an IRGPN of the invention, or a portion of any of these nucleotide sequences. A polynucleotide molecule which is complementary to such a nucleotide sequence is one which is sufficiently complementary to the nucleotide sequence such that it can hybridize to the nucleotide sequence, thereby forming a stable duplex.

The polynucleotide molecule of the invention, moreover, can comprise only a portion of the polynucleotide sequence of an IRG, for example, a fragment which can be used as a probe or primer. The probe/primer typically comprises a substantially purified oligonucleotide. The oligonucleotide typically comprises a region of nucleotide sequence that hybridizes under stringent conditions to at least about 7 or 15, preferably about 25, more preferably about 50, 75, 100, 125, 150, 175, 200, 225, 250, 275, 300, 325, 350, 400 or more consecutive nucleotides of an IRG or an IRGPN of the invention.

Probes based on the nucleotide sequence of an IRG or an IRGPN of the invention can be used to detect transcripts or genomic sequences corresponding to the IRG or IRGPN of the invention. In preferred embodiments, the probe comprises a label group attached thereto, e.g., the label group can be a radioisotope, a fluorescent compound, an enzyme, or an enzyme co-factor. Such probes can be used as a part of a diagnostic kit for identifying cells or tissue which misexpress (e.g., over- or under-express) an IRG, or which have greater or fewer copies of an IRG. For example, a level of an IRG product in a sample of cells from a subject may be determined, or the presence of mutations or deletions of an IRG of the invention may be assessed.

The invention further encompasses polynucleotide molecules that differ from the polynucleotide sequences of the IRGs listed in Table 3 but encode the same proteins as those encoded by the genes shown in Table 3 due to degeneracy of the genetic code.

The invention also specifically encompasses homologs of the IRGs listed in Table 3 of other species. Gene homologs are well understood in the art and are available using databases or search engines such as the Pubmed-Entrez database.

The invention also encompasses polynucleotide molecules which are structurally different from the molecules described above (i.e., which have a slight altered sequence), but which have substantially the same properties as the molecules above (e.g., encoded amino acid sequences, or which are changed only in non-essential amino acid residues). Such molecules include allelic variants, and are described in greater detail in subsections herein.

In addition to the nucleotide sequences of the IRGs listed in Table 3, it will be appreciated by those skilled in the art that DNA sequence polymorphisms that lead to changes in the amino acid sequences of the proteins encoded by the IRGs listed in Table 3 may exist within a population (e.g., the human population). Such genetic polymorphism in the IRGs listed in Table 3 may exist among individuals within a population due to natural allelic variation. An allele is one of a group of genes which occur alternatively at a given genetic locus. In addition it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus or to a polypeptide encoded by the nucleotide sequence.

Polynucleotide molecules corresponding to natural allelic variants and homologs of the IRGs can be isolated based on their homology to the IRGs listed in Table 3, using the cDNAs disclosed herein, or a portion thereof, as a hybridization probe according to standard hybridization techniques under stringent hybridization conditions. Polynucleotide molecules corresponding to natural allelic variants and homologs of the IRGs of the invention can further be isolated by mapping to the same chromosome or locus as the IRGs of the invention.

In another embodiment, an isolated polynucleotide molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes under stringent conditions to a polynucleotide molecule corresponding to a nucleotide sequence of an IRG of the invention. Preferably, the isolated polynucleotide molecule of the invention hybridizes under stringent conditions to the sequence of one of the IRGs set forth in Table 3, or corresponds to a naturally-occurring polynucleotide molecule.

In addition to naturally-occurring allelic variants of the IRG of the invention that may exist in the population, the skilled artisan will further appreciate that changes can be introduced by mutation into the nucleotide sequences of the IRGs of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

In yet other aspects of the invention, polynucleotides of a IRG may comprise one or more mutations. An isolated polynucleotide molecule encoding a protein with a mutation in an IRGPP of the invention can be created by introducing one or more nucleotide substitutions, additions or deletions into the nucleotide sequence of the gene encoding the IRGPP, such that one or more amino acid substitutions, additions or deletions are introduced into the encoded protein. Such techniques are well known in the art. Mutations can be introduced into the IRG of the invention by standard techniques, such as site-directed mutagenesis and PCR-mediated mutagenesis. Preferably, conservative amino acid substitutions are made at one or more predicted non-essential amino acid residues. Alternatively, mutations can be introduced randomly along all or part of a coding sequence of a IRG of the invention, such as by saturation mutagenesis, and the resultant mutants can be screened for biological activity to identify mutants that retain activity. Following mutagenesis, the encoded protein can be expressed recombinantly and the activity of the protein can be determined.

A polynucleotide may be further modified to increase stability in vivo. Possible modifications include, but are not limited to, the addition of flanking sequences at the 5′ and/or 3′ ends; the use of phosphorothioate or 20-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Another aspect of the invention pertains to isolated polynucleotide molecules, which are antisense to the IRGs of the invention. An “antisense” polynucleotide comprises a nucleotide sequence which is complementary to a “sense” polynucleotide encoding a protein, e.g., complementary to the coding strand of a double-stranded cDNA molecule or complementary to an mRNA sequence. Accordingly, an antisense polynucleotide can hydrogen bond to a sense polynucleotide. The antisense polynucleotide can be complementary to an entire coding strand of a gene of the invention or to only a portion thereof. In one embodiment, an antisense polynucleotide molecule is antisense to a “coding region” of the coding strand of a nucleotide sequence of the invention. The term “coding region” includes the region of the nucleotide sequence comprising codons which are translated into amino acids. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a gene of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenosine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation (i.e., RNA transcribed from the inserted polynucleotide will be of an antisense orientation to a target polynucleotide of interest, described further in the following subsection).

The antisense polynucleotide molecules of the invention are typically administered to a subject or generated in situ such that they hybridize with or bind to cellular mRNA and/or genomic DNA encoding an IRGPP of the invention to thereby inhibit expression of the protein, e.g., by inhibiting transcription and/or translation. The hybridization can be by conventional nucleotide complementarity to form a stable duplex or, for example, in the cases of an antisense polynucleotide molecule which binds to DNA duplexes, through specific interactions in the major groove of the double helix. An example of a route of administration of antisense polynucleotide molecules of the invention include direct injection at a tissue site. Alternatively, antisense polynucleotide molecules can be modified to target selected cells and then administered systemically. For example, for systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intracellular concentrations of the antisense molecules, vector constructs comprising the antisense polynucleotide molecules are preferably placed under the control of a strong promoter.

In yet another embodiment, the antisense polynucleotide molecule of the invention is an -anomeric polynucleotide molecule. An -anomeric polynucleotide molecule forms specific double-stranded hybrids with complementary RNA in which, contrary to the usual-units, the strands run parallel to each other. The antisense polynucleotide molecule can also comprise a 2′-o-methylribonucleotide or a chimeric RNA-DNA analogue.

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes) can be used to catalytically cleave mRNA transcripts of the IRGs of the invention to thereby inhibit translation of the mRNA. A ribozyme having specificity for an IRGPN can be designed based upon the nucleotide sequence of the IRGPN. Alternatively, mRNA transcribed from an IRG can be used to select a catalytic RNA having a specific ribonuclease activity from a pool of RNA molecules. Alternatively, expression of an IRG of the invention can be inhibited by targeting nucleotide sequences complementary to the regulatory region of the IRG (e.g., the promoter and/or enhancers) to form triple helical structures that prevent transcription of the gene in target cells.

Expression of the IRGs of the invention can also be inhibited using RNA interference (“RNAi”). This is a technique for post-transcriptional gene silencing (“PTGS”), in which target gene activity is specifically abolished with cognate double-stranded RNA (“dsRNA”). RNAi involves a process in which the dsRNA is cleaved into 23 bp short interfering RNAs (siRNAs) by an enzyme called Dicer (Hamilton & Baulcombe, Science 286:950, 1999), thus producing multiple “trigger” molecules from the original single dsRNA. The siRNA-Dicer complex recruits additional components to form an RNA-induced Silencing Complex (RISC) in which the unwound siRNA base pairs with complementary mRNA, thus guiding the RNAi machinery to the target mRNA resulting in the effective cleavage and subsequent degradation of the mRNA (Hammond et al., Nature 404: 293-296, 2000; Zamore et al., Cell 101: 25-33; 2000; Pham et al., Cell 117: 83-94, 2004). In this way, the activated RISC could potentially target multiple mRNAs, and thus function catalytically.

RNAᵢ technology is disclosed, for example, in U.S. Pat. No. 5,919,619 and PCT Publication Nos. WO99/14346 and WO01/29058. Typically, dsRNA of about 21 nucleotides, homologous to the target gene, is introduced into the cell and a sequence specific reduction in gene activity is observed.

In yet another embodiment, the polynucleotide molecules of the present invention can be modified at the base moiety, sugar moiety or phosphate backbone to improve the stability, hybridization, or solubility of the molecule. For example, the deoxyribose phosphate backbone of the polynucleotide molecules can be modified to generate peptide polynucleotides. As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols.

PNAs can be used in therapeutic and diagnostic applications. For example, PNAs can be used as antisense agents for sequence-specific modulation of IRG expression by, for example, inducing transcription or translation arrest or inhibiting replication. PNAs of the polynucleotide molecules of the invention can be used in the analysis of single base pair mutations in a gene, (e.g., by PNA-directed PCR clamping). They may also serve as artificial restriction enzymes when used in combination with other enzymes (e.g., S1 nucleases) or as probes or primers for DNA sequencing or hybridization.

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated which may combine the advantageous properties of PNA and DNA. Such chimeras allow DNA recognition enzymes, (e.g., DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation. The synthesis of PNA-DNA chimeras can be performed. For example, a DNA chain can be synthesized on a solid support using standard phosphoramidite coupling chemistry. Modified nucleoside analogs, such as 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, the oligonucleotide may include other appended groups such as peptides (e.g., for targeting host cell receptors in vivo), or agents facilitating transport across the cell membrane or the blood-kidney barrier (see, e.g. PCT Publication No. WO 89/10134). In addition, oligonucleotides can be modified with hybridization-triggered cleavage agents or intercalating agents. To this end, the oligonucleotide may be conjugated to another molecule (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label).

### Isolated Polypeptides

Several aspects of the invention pertain to isolated IRGPPs, and biologically active portions thereof, as well as polypeptide fragments suitable for use as immunogens to raise anti-IRGPP antibodies. In one embodiment, native IRGPPs can be isolated from cells or tissue sources by an appropriate purification scheme using standard protein purification techniques. Standard purification methods include electrophoretic, molecular, immunological and chromatographic techniques, including ion exchange, hydrophobic, affinity, and reverse-phase HPLC chromatography, and chromatofocusing. For example, an IRGPP may be purified using a standard anti-IRGPP antibody column. Ultrafiltration and diafiltration techniques, in conjunction with protein concentration, are also useful. The degree of purification necessary will vary depending on the use of the IRGPP. In some instances no purification will be necessary.

In another embodiment, IRGPPs or mutated IRGPPs are produced by recombinant DNA techniques. Alternative to recombinant expression, an IRGPP or mutated IRGPP can be synthesized chemically using standard peptide synthesis techniques.

The invention also provides variants of IRGPPs. The variant of an IRGPP is substantially homologous to the native IRGPP encoded by an IRG listed in Table 3, and retains the functional activity of the native IRGPP, yet differs in amino acid sequence due to natural allelic variation or mutagenesis, as described in detail above. Accordingly, in another embodiment, the variant of an IRGPP is a protein which comprises an amino acid sequence at least about 60%, 65%, 70%, 75%, 80%, 85%, 90%, 95%, 98% or more homologous to the amino acid sequence of the original IRGPP.

In a non-limiting example, as used herein, proteins are referred to as “homologs” and “homologous” where a first protein region and a second protein region are compared in terms of identity. To determine the percent identity of two amino acid sequences or of two polynucleotide sequences, the sequences are aligned for optimal comparison purposes (e.g., gaps can be introduced in one or both of a first and a second amino acid or polynucleotide sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleotide “identity” is equivalent to amino acid or nucleotide “homology”). The percent identity between the two sequences is a function of the number of identical positions shared by the sequences, taking into account the number of gaps and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453, 1970) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6.

The polynucleotide and protein sequences of the present invention can further be used as a “query sequence” to perform a search against public databases to, for example, identify other family members or related sequences. Such searches can be performed using BLAST programs available at the BLAST website maintained by the National Center of biotechnology Information (NCBI), National Library of Medicine, Washington D.C. USA.

The invention also provides chimeric or fusion IRGPPs. Within a fusion IRGPP the polypeptide can correspond to all or a portion of an IRGPP. In a preferred embodiment, a fusion IRGPP comprises at least one biologically active portion of an IRGPP. Within the fusion protein, the term “operatively linked” is intended to indicate that the IRGPP-related polypeptide and the non-IRGPP-related polypeptide are fused in-frame to each other. The non-IRGPP-related polypeptide can be fused to the N-terminus or C-terminus of the IRGPP-related polypeptide.

A peptide linker sequence may be employed to separate the IRGPP-related polypeptide from non-IRGPP-related polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the IRGPP-related polypeptide and non-IRGPP-related polypeptide; and (3) the lack of hydrophobic or charged residues that might react with the polypeptide functional epitopes. Preferred peptide linker sequences contain gly, asn and ser residues. Other near neutral amino acids, such as thr and ala may also be used in the linker sequence. Amino acid sequences which may be used as linkers are well known in the art. The linker sequence may generally be from 1 to about 50 amino acids in length. Linker sequences are not required when the IRGPP-related polypeptide and non-IRGPP-related polypeptide have non-essential N-terminal amino acid regions that can be used to separate the functional domains and prevent steric interference.

For example, in one embodiment, the fusion protein is a glutathione S-transferase (GST)-IRGPP fusion protein in which the IRGPP sequences are fused to the C-terminus of the GST sequences. Such fusion proteins can facilitate the purification of recombinant IRGPPs.

The IRGPP-fusion proteins of the invention can be incorporated into pharmaceutical compositions and administered to a subject in vivo, as described herein. The IRGPP-fusion proteins can be used to affect the bioavailability of an IRGPP substrate. IRGPP-fusion proteins may be useful therapeutically for the treatment of, or prevention of, damages caused by, for example, (i) aberrant modification or mutation of an IRG; (ii) mis-regulation of an IRG; and (iii) aberrant post-translational modification of an IRGPP.

Moreover, the IRGPP-fusion proteins of the invention can be used as immunogens to produce anti-IRGPP antibodies in a subject, to purify IRGPP ligands, and to identify molecules which inhibit the interaction of an IRGPP with an IRGPP substrate in screening assays.

Preferably, an IRGPP-chimeric or fusion protein of the invention is produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). An IRGPP-encoding polynucleotide can be cloned into such an expression vector such that the fusion moiety is linked in-frame to the IRGPP.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products).

In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the IRGPPs of the invention which function as either agonists or as antagonists to the IRGPPs. In one embodiment, antagonists or agonists of IRGPPs are used as therapeutic agents. For example, antagonists of an up-regulated IRG that can decrease the activity or expression of such a gene and therefore ameliorate influenza in a subject wherein the IRG is abnormally increased in level or activity. In this embodiment, treatment of such a subject may comprise administering an antagonist wherein the antagonist provides decreased activity or expression of the targeted IRG.

In certain embodiments, an agonist of the IRGPPs can retain substantially the same, or a subset, of the biological activities of the naturally occurring form of an IRGPP or may enhance an activity of an IRGPP. In certain embodiments, an antagonist of an IRGPP can inhibit one or more of the activities of the naturally occurring form of the IRGPP by, for example, competitively modulating an activity of an IRGPP. Thus, specific biological effects can be elicited by treatment with a variant of limited function. In one embodiment, treatment of a subject with a variant having a subset of the biological activities of the naturally occurring forth of the protein has fewer side effects in a subject relative to treatment with the naturally occurring form of the IRGPP.

Mutants of an IRGPP which function as either IRGPP agonists or as IRGPP antagonists can be identified by screening combinatorial libraries of mutants, e.g., truncation mutants, of an IRGPP for IRGPP agonist or antagonist activity. In certain embodiments, such mutants may be used, for example, as a therapeutic protein of the invention. A diverse library of IRGPP mutants can be produced by, for example, enzymatically ligating a mixture of synthetic oligonucleotides into gene sequences such that a degenerate set of potential IRGPP sequences is expressible as individual polypeptides, or alternatively, as a set of larger fusion proteins (e.g., for phage display) containing the set of IRGPP sequences therein. There are a variety of methods which can be used to produce libraries of potential IRGPP variants from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential IRGPP sequences. Methods for synthesizing degenerate oligonucleotides are known in the art.

In addition, libraries of fragments of a protein coding sequence corresponding to an IRGPP of the invention can be used to generate a diverse or heterogenous population of IRGPP fragments for screening and subsequent selection of variants of an IRGPP. In one embodiment, a library of coding sequence fragments can be generated by treating a double-stranded PCR fragment of an IRGPP coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, an expression library can be derived which encodes N-terminal, C-terminal and internal fragments of various sizes of the IRGPP.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify IRGPP variants (Delgrave et al. Protein Engineering 6:327-331, 1993).

Portions of an IRGPP or variants of an IRGPP having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Methods and compositions for screening for protein inhibitors or activators are known in the art (see U.S. Pat. Nos. 4,980,281, 5,266,464, 5,688,635, and 5,877,007, which are incorporated herein by reference).

It is contemplated in the present invention that IRGPPs are cleaved into fragments for use in further structural or functional analysis, or in the generation of reagents such as IRGPP and IRGPP-specific antibodies. This can be accomplished by treating purified or unpurified polypeptide with a proteolytic enzyme (i.e., a proteinase) including, but not limited to, serine proteinases (e.g., chymotrypsin, trypsin, plasmin, elastase, thrombin, substilin) metal proteinases (e.g., carboxypeptidase A, carboxypeptidase B, leucine aminopeptidase, thermolysin, collagenase), thiol proteinases (e.g., papain, bromelain, Streptococcal proteinase, clostripain) and/or acid proteinases (e.g., pepsin, gastricsin, trypsinogen). Polypeptide fragments are also generated using chemical means such as treatment of the polypeptide with cyanogen bromide (CNBr), 2-nitro-5-thiocyanobenzoic acid, isobenzoic acid, BNPA-skatole, hydroxylamine or a dilute acid solution. Recombinant techniques are also used to produce specific fragments of an IRGPP.

In addition, the invention also contemplates that compounds sterically similar to a particular IRGPP may be formulated to mimic the key portions of the peptide structure, called peptidomimetics or peptide mimetics. Mimetics are peptide-containing molecules which mimic elements of polypeptide secondary structure. See, for example, U.S. Pat. No. 5,817,879 (incorporated by reference hereinafter in its entirety). The underlying rationale behind the use of peptide mimetics is that the peptide backbone of polypeptides exists chiefly to orient amino acid side chains in such a way as to facilitate molecular interactions, such as those of receptor and ligand. Recently, peptide and glycoprotein mimetic antigens have been described which elicit protective antibody to Neisseria meningitidis serogroup B, thereby demonstrating the utility of mimetic applications (Moe et al., Int. Rev. Immunol. 20:201-20, 2001; Berezin et al., J Mol. Neurosci. 22:33-39, 2004). Successful applications of the peptide mimetic concept have thus far focused on mimetics of b-turns within polypeptides. Likely b-turn structures within an IRGPP can be predicted by computer-based algorithms. For example, U.S. Pat. No. 5,933,819, incorporated by reference hereinafter in its entirety, describes a neural network based method and system for identifying relative peptide binding motifs from limited experimental data. In particular, an artificial neural network (ANN) is trained with peptides with known sequence and function (i.e., binding strength) identified from a phage display library. The ANN is then challenged with unknown peptides, and predicts relative binding motifs. Analysis of the unknown peptides validate the predictive capability of the ANN. Once the component amino acids of the turn are determined, mimetics can be constructed to achieve a similar spatial orientation of the essential elements of the amino acid side chains, as discussed in U.S. Pat. No. 6,420,119 and U.S. Pat. No. 5,817,879, and in Kyte and Doolittle, J. Mol. Biol., 157:105-132, 1982; Moe and Granoff, Int. Rev. Immunol., 20:201-20, 2001; and Granoff et al., J. Immunol., 167:6487-96, 2001, each is incorporated by reference hereinafter in its entirety.

### Antibodies

In another aspect, the invention includes antibodies that are specific to IRGPPs of the invention or their variants. Preferably the antibodies are monoclonal, and most preferably, the antibodies are humanized, as per the description of antibodies described below.

An isolated IRGPP, or a portion or fragment thereof, can be used as an immunogen to generate antibodies that bind the IRGPP using standard techniques for polyclonal and monoclonal antibody preparation. A full-length IRGPP can be used or, alternatively, the invention provides antigenic peptide fragments of the IRGPP for use as immunogens. The antigenic peptide of an IRGPP comprises at least 8 amino acid residues of an amino acid sequence encoded by an IRG set forth in Table 3 or an homolog thereof, and encompasses an epitope of an IRGPP such that an antibody raised against the peptide forms a specific immune complex with the IRGPP. Preferably, the antigenic peptide comprises at least 8 amino acid residues, more preferably at least 12 amino acid residues, even more preferably at least 16 amino acid residues, and most preferably at least 20 amino acid residues.

Immunogenic portions (epitopes) may generally be identified using well known techniques. Such techniques include screening polypeptides for the ability to react with antigen-specific antibodies, antisera and/or T-cell lines or clones. As used herein, antisera and antibodies are “antigen-specific” if they bind to an antigen with a binding affinity equal to, or greater than 10⁵ M⁻¹. Such antisera and antibodies may be prepared as described herein, and using well known techniques. An epitope of an IRGPP is a portion that reacts with such antisera and/or T-cells at a level that is not substantially less than the reactivity of the full length polypeptide (e.g., in an ELISA and/or T-cell reactivity assay). Such epitopes may react within such assays at a level that is similar to or greater than the reactivity of the full length polypeptide. Such screens may generally be performed using methods well known to those of ordinary skill in the art. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Preferred epitopes encompassed by the antigenic peptide are regions of the IRGPP that are located on the surface of the protein, e.g., hydrophilic regions, as well as regions with high antigenicity.

An IRGPP immunogen typically is used to prepare antibodies by immunizing a suitable subject, (e.g., rabbit, goat, mouse or other mammal) with the immunogen. An appropriate immunogenic preparation can contain, for example, recombinantly expressed IRGPP or a chemically synthesized IRGPP. The preparation can further include an adjuvant, such as Freund's complete or incomplete adjuvant, or a similar immunostimulatory agent. Immunization of a suitable subject with an immunogenic IRGPP preparation induces a polyclonal anti-IRGPP antibody response. Techniques for preparing, isolating and using antibodies are well known in the art.

Accordingly, another aspect of the invention pertains to monoclonal or polyclonal anti-IRGPP antibodies and immunologically active portions of the antibody molecules, including F(ab) and F(ab′)₂ fragments which can be generated by treating the antibody with an enzyme such as pepsin.

Polyclonal anti-IRGPP antibodies can be prepared as described above by immunizing a suitable subject with an IRGPP. The anti-IRGPP antibody titer in the immunized subject can be monitored over time by standard techniques, such as with an enzyme linked immunosorbent assay (ELISA) using immobilized IRGPP. If desired, the antibody molecules directed against IRGPPs can be isolated from the subject (e.g., from the blood) and further purified by well known techniques, such as protein A chromatography, to obtain the IgG fraction. At an appropriate time after immunization, e.g., when the anti-IRGPP antibody titers are highest, antibody-producing cells can be obtained from the subject and used to prepare monoclonal antibodies by standard techniques, such as the hybridoma technique, human B cell hybridoma technique, the EBV-hybridoma technique, or trioma techniques. The technology for producing monoclonal antibody hybridomas is well known. Briefly, an immortal cell line (typically a myeloma) is fused to lymphocytes (typically splenocytes) from a mammal immunized with an IRGPP immunogen as described above, and the culture supernatants of the resulting hybridoma cells are screened to identify a hybridoma producing a monoclonal antibody that binds to an IRGPP of the invention. Any of the many well known protocols used for fusing lymphocytes and immortalized cell lines can be applied for the purpose of generating an anti-IRGPP monoclonal antibody. Moreover, the ordinarily skilled worker will appreciate that there are many variations of such methods which also would be useful.

Alternative to preparing monoclonal antibody-secreting hybridomas, a monoclonal anti-IRGPP antibody can be identified and isolated by screening a recombinant combinatorial immunoglobulin library (e.g., an antibody phase display library) with IRGPP to thereby isolate immunoglobulin library members that bind to an IRGPP. Kits for generating and screening phage display libraries are commercially available.

The anti-IRGPP antibodies also include “Single-chain Fv” or “scFv” antibody fragments. The scFv fragments comprise the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding.

Additionally, recombinant anti-IRGPP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art (see e.g., U.S. Pat. Nos. 6,677,436 and 6,808,901).

Humanized antibodies are particularly desirable for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies), which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody will preferably also comprise at least a portion of an immunoglobulin constant region (Fc), typically that of a human immunoglobulin.

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a polypeptide corresponding to an IRGPP of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope.

In a preferred embodiment, the antibodies to IRGPP are capable of reducing or eliminating the biological function of IRGPP, as is described below. That is, the addition of anti-IRGPP antibodies (either polyclonal or preferably monoclonal) to IRGPP (or cells containing IRGPP) may reduce or eliminate the IRGPP activity. Generally, at least a 25% decrease in activity is preferred, with at least about 50% being particularly preferred and about a 95-100% decrease being especially preferred.

An anti-IRGPP antibody can be used to isolate an IRGPP of the invention by standard techniques, such as affinity chromatography or immunoprecipitation. An anti-IRGPP antibody can facilitate the purification of natural IRGPPs from cells and of recombinantly produced IRGPPs expressed in host cells. Moreover, an anti-IRGPP antibody can be used to detect an IRGPP (e.g., in a cellular lysate or cell supernatant on the cell surface) in order to evaluate the abundance and pattern of expression of the IRGPP. Anti-IRGPP antibodies can be used diagnostically to monitor protein levels in tissue as part of a clinical testing procedure, for example, to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive materials include 125I, 131I, 35S or 3H.

Anti-IRGPP antibodies of the invention are also useful for targeting a therapeutic to a cell or tissue comprising the antigen of the anti-IRGPP antibody. A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

As is well known in the art, a given polypeptide or polynucleotide may vary in its immunogenicity. It is often necessary therefore to couple the immunogen (e.g., a polypeptide or polynucleotide) of the present invention with a carrier. Exemplary and preferred carriers are CRM197, E. coli (LT) toxin, V. cholera (CT) toxin, keyhole limpet hemocyanin (KLH) and bovine serum albumin (BSA). Other albumins such as ovalbumin, mouse serum albumin or rabbit serum albumin can also be used as carriers.

Where an IRGPP (or a fragment thereof) and a carrier protein are conjugated (i.e., covalently associated), conjugation may be any chemical method, process or genetic technique commonly used in the art. For example, an IRGPP (or a fragment thereof) and a carrier protein, may be conjugated by techniques, including, but not limited to: (1) direct coupling via protein functional groups (e.g., thiol-thiol linkage, amine-carboxyl linkage, amine-aldehyde linkage; enzyme direct coupling); (2) homobifunctional coupling of amines (e.g., using bis-aldehydes); (3) homobifunctional coupling of thiols (e.g., using bis-maleimides); (4) homobifunctional coupling via photoactivated reagents (5) heterobifunctional coupling of amines to thiols (e.g., using maleimides); (6) heterobifunctional coupling via photoactivated reagents (e.g., the -carbonyldiazo family); (7) introducing amine-reactive groups into a poly- or oligosaccharide via cyanogen bromide activation or carboxymethylation; (8) introducing thiol-reactive groups into a poly- or oligosaccharide via a heterobifunctional compound such as maleimido-hydrazide; (9) protein-lipid conjugation via introducing a hydrophobic group into the protein and (10) protein-lipid conjugation via incorporating a reactive group into the lipid. Also, contemplated are heterobifunctional “non-covalent coupling” techniques such the Biotin-Avidin interaction. For a comprehensive review of conjugation techniques, see Aslam and Dent (Aslam and Dent, “Bioconjugation: Protein Coupling Techniques for the Biomedical Sciences,” Macmillan Reference Ltd., London, England, 1998), incorporated hereinafter by reference in its entirety.

In a specific embodiment, antibodies to an IRGPP may be used to eliminate the IRGPP in vivo by activating the complement system or mediating antibody-dependent cellular cytotoxicity (ADCC), or cause uptake of the antibody coated cells by the receptor-mediated endocytosis (RE) system.

### Vectors

Another aspect of the invention pertains to vectors containing a polynucleotide encoding an IRGPP, a variant of an IRGPP, or a portion thereof. One type of vector is a “plasmid,” which includes a circular double-stranded DNA loop into which additional DNA segments can be ligated. In the present specification, “plasmid” and “vector” can be used interchangeably as the plasmid is the most commonly used form of vector. Vectors also include expression vectors and gene delivery vectors.

The expression vectors of the invention comprise a polynucleotide encoding an IRGPP or a portion thereof in a form suitable for expression of the polynucleotide in a host cell, which means that the expression vectors include one or more regulatory sequences, selected on the basis of the host cells to be used for expression, and operatively linked to the polynucleotide sequence to be expressed. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, such as IRGPPs, mutant forms of IRGPPs, IRGPP-fusion proteins, and the like.

The expression vectors of the invention can be designed for expression of IRGPPs in prokaryotic or eukaryotic cells. For example, IRGPPs can be expressed in bacterial cells such as E. coli, insect cells (using baculovirus expression vectors), yeast cells or mammalian cells. Alternatively, the expression vector can be transcribed and translated in vitro, for example using T7 promoter regulatory sequences and T7 polymerase.

The expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of the recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Purified fusion proteins can be utilized in IRGPP activity assays, (e.g., direct assays or competitive assays described in detail below), or to generate antibodies specific for IRGPPs.

One strategy to maximize recombinant protein expression in E. coli is to express the protein in a host bacteria with an impaired capacity to proteolytically cleave the recombinant protein. Another strategy is to alter the polynucleotide sequence of the polynucleotide to be inserted into an expression vector so that the individual codons for each amino acid are those preferentially utilized in E. coli. Such alteration of polynucleotide sequences of the invention can be carried out by standard DNA synthesis techniques.

In another embodiment, the IRGPP expression vector is a yeast expression vector. Examples of vectors for expression in yeast S. cerevisiae include pYepSec1, pMFa, pJRY188, pYES2 and picZ (Invitrogen Corp, San Diego, Calif.).

Alternatively, IRGPPs of the invention can be expressed in insect cells using baculovirus expression vectors. Baculovirus vectors available for expression of proteins in cultured insect cells (e.g., Sf9 cells) include the pAc series and the pVL series.

In yet another embodiment, a polynucleotide of the invention is expressed in mammalian cells using a mammalian expression vector. When used in mammalian cells, the expression vector's control functions are often provided by viral regulatory elements. For example, commonly used promoters are derived from polyoma, adenovirus 2 and 5, cytomegalovirus and Simian Virus 40.

In another embodiment, the mammalian expression vector is capable of directing expression of the polynucleotide preferentially in a particular cell type (e.g., tissue-specific regulatory elements are used to express the polynucleotide). Tissue-specific regulatory elements are known in the art and may include epithelial cell-specific promoters. Other non-limiting examples of suitable tissue-specific promoters include the liver-specific promoter (e.g., albumin promoter), lymphoid-specific promoters, promoters of T cell receptors and immunoglobulins, neuron-specific promoters (e.g., the neurofilament promoter), pancreas-specific promoters (e.g., insulin promoter), and mammary gland-specific promoters (e.g., milk whey promoter). Developmentally-regulated promoters (e.g., the -fetoprotein promoter) are also encompassed.

The invention also provides a recombinant expression vector comprising a polynucleotide encoding an IRGPP cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to an IRG of the invention. Regulatory sequences operatively linked to a polynucleotide cloned in the antisense orientation can be chosen which direct the continuous expression of the antisense RNA molecule in a variety of cell types, for instance, viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region, the activity of which can be determined by the cell type into which the vector is introduced.

The invention further provides gene delivery vehicles for delivery of polynucleotides to cells, tissues, or a mammal for expression. For example, a polynucleotide sequence of the invention can be administered either locally or systemically in a gene delivery vehicle. These constructs can utilize viral or non-viral vector approaches in in vivo or ex vivo modality. Expression of the coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle is preferably a viral vector and, more preferably, a retroviral, lentiviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, togavirus viral vector.

The delivery of gene therapy constructs of this invention into cells is not limited to the above mentioned viral vectors. Other delivery methods and media may be employed such as, for example, nucleic acid expression vectors, polycationic condensed DNA linked or unlinked to killed adenovirus alone, ligand linked DNA, liposomes, eukaryotic cell delivery vehicles cells, deposition of photopolymerized hydrogel materials, handheld gene transfer particle gun, ionizing radiation, nucleic charge neutralization or fusion with cell membranes. Particle mediated gene transfer may be employed. Briefly, DNA sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed.

Another aspect of the invention pertains to the expression of IRGPPs using a regulatable expression system. Examples of regulatable systems include the Tet-on/off system of BD Biosciences (San Jose, Calif.), the ecdysone system of Invitrogen (Carlsbad, Calif., the mifepristone/progesterone system of Valentis (Burlingame, Calif.), and the rapamycin system of Ariad (Cambridge, Mass.).

### Immunogens and Immunogenic Compositions

Within certain aspects, IRGPP, IRGPN, IRGPP-specific T cell, IRGPP-presenting APC, IRG-containing vectors, including but are not limited to expression vectors and gene delivery vectors, may be utilized as vaccines for influenza. Vaccines may comprise one or more such compounds/cells and an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated). Vaccines within the scope of the present invention may also contain other compounds, which may be biologically active or inactive. For example, one or more immunogenic portions of other antigens may be present, either incorporated into a fusion polypeptide or as a separate compound, within the composition of vaccine.

A vaccine may contain DNA encoding one or more IRGPP or portion of IRGPP, such that the polypeptide is generated in situ. As noted above, the DNA may be present within any of a variety of delivery systems known to those of ordinary skill in the art, including nucleic acid expression vectors, gene delivery vectors, and bacteria expression systems. Numerous gene delivery techniques are well known in the art. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Any of a variety of immunostimulants may be employed in the vaccines of this invention. For example, an adjuvant may be included. As defined previously, an “adjuvant” is a substance that serves to enhance the immunogenicity of an antigen. Thus, adjuvants are often given to boost the immune response and are well known to the skilled artisan. Examples of adjuvants contemplated in the present invention include, but are not limited to, aluminum salts (alum) such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-Tetradecanoyloxytetradecanoylamino]ethyl 2-Deoxy-4-O-phosphono-3-O—[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, MPL™ (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646), polypeptides, saponins such as Quil A or STIMULON™ QS-21 (Antigenics, Framingham, Mass.), described in U.S. Pat. No. 5,057,540, a pertussis toxin (PT), or an E. coli heat-labile toxin (LT), particularly LT-K63, LT-R72, CT-S109, PT-K9/G129; see, e.g., International Patent Publication Nos. WO 93/13302 and WO 92/19265, cholera toxin (either in a wild-type or mutant form, e.g., wherein the glutamic acid at amino acid position 29 is replaced by another amino acid, preferably a histidine, in accordance with published International Patent Application number WO 00/18434). Various cytokines and lymphokines are suitable for use as adjuvants. One such adjuvant is granulocyte-macrophage colony stimulating factor (GM-CSF), which has a nucleotide sequence as described in U.S. Pat. No. 5,078,996. A plasmid containing GM-CSF cDNA has been transformed into E. coli and has been deposited with the American Type Culture Collection (ATCC), 1081 University Boulevard, Manassas, Va. 20110-2209, under Accession Number 39900. The cytokine IL-12 is another adjuvant which is described in U.S. Pat. No. 5,723,127. Other cytokines or lymphokines have been shown to have immune modulating activity, including, but not limited to, the interleukins 1-alpha, 1-beta, 2, 4, 5, 6, 7, 8, 10, 13, 14, 15, 16, 17 and 18, the interferons-alpha, beta and gamma, granulocyte colony stimulating factor, and the tumor necrosis factors alpha and beta, and are suitable for use as adjuvants.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO 94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within vaccines to facilitate production of an antigen-specific immune response that targets cancer cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-influenza effects per se and/or to be immunologically compatible with the receiver (i.e., matched HLA haplotype). APCs may generally be isolated from any of a variety of biological fluids and organs, and may be autologous, allogeneic, syngeneic or xenogenic cells.

Vaccines may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine may be stored in a freeze-dried condition requiring only the addition of a sterile liquid carrier immediately prior to use.

### Screening Methods

The invention also provides methods (also referred to herein as “screening assays”) for identifying modulators, i.e., candidate or test compounds or agents comprising therapeutic moieties (e.g., peptides, peptidomimetics, peptoids, polynucleotides, small molecules or other drugs) which (a) bind to an IRGPP, or (b) have a modulatory (e.g., stimulatory or inhibitory) effect on the activity of an IRGPP or, more specifically, (c) have a modulatory effect on the interactions of the IRGPP with one or more of its natural substrates (e.g., peptide, protein, hormone, co-factor, or polynucleotide), or (d) have a modulatory effect on the expression of the IRGPPs. Such assays typically comprise a reaction between the IRGPP and one or more assay components. The other components may be either the test compound itself, or a combination of the test compound and a binding partner of the IRGPP.

To screen for compounds which interfere with binding of two proteins e.g., an IRGPP and its binding partner, a Scintillation Proximity Assay can be used. In this assay, the IRGPP is labeled with an isotope such as ¹²⁵I. The binding partner is labeled with a scintillant, which emits light when proximal to radioactive decay (i.e., when the IRGPP is bound to its binding partner). A reduction in light emission will indicate that a compound has interfered with the binding of the two proteins.

Alternatively a Fluorescence Energy Transfer (FRET) assay could be used. In a FRET assay of the invention, a fluorescence energy donor is comprised on one protein (e.g., an IRGPP) and a fluorescence energy acceptor is comprised on a second protein (e.g., a binding partner of the IRGPP). If the absorption spectrum of the acceptor molecule overlaps with the emission spectrum of the donor fluorophore, the fluorescent light emitted by the donor is absorbed by the acceptor. The donor molecule can be a fluorescent residue on the protein (e.g., intrinsic fluorescence such as a tryptophan or tyrosine residue), or a fluorophore which is covalently conjugated to the protein (e.g., fluorescein isothiocyanate, FITC). An appropriate donor molecule is then selected with the above acceptor/donor spectral requirements in mind.

Thus, in this example, an IRGPP is labeled with a fluorescent molecule (i.e., a donor fluorophore) and its binding partner is labeled with a quenching molecule (i.e., an acceptor). When the IRGPP and its binding partner are bound, fluorescence emission will be quenched or reduced relative the IRGPP alone. Similarly, a compound which can dissociate the interaction of the IRGPP-partner complex, will result in an increase in fluorescence emission, which indicates the compound has interfered with the binding of the IRGPP to its binding partner.

Another assay to detect binding or dissociation of two proteins is fluorescence polarization or anisotropy. In this assay, the investigated protein (e.g., an IRGPP) is labeled with a fluorophore with an appropriate fluorescence lifetime. The protein sample is then excited with vertically polarized light. The value of anisotropy is then calculated by determining the intensity of the horizontally and vertically polarized emission light. Next, the labeled protein (IRGPP) is mixed with an IRGPP binding partner and the anisotropy measured again. Because fluorescence anisotropy intensity is related to the rotational freedom of the labeled protein, the more rapidly a protein rotates in solution, the smaller the anisotropy value. Thus, if the labeled IRGPP is part of a complex (e.g., IRGPP-partner), the IRGPP rotates more slowly in solution (relative to free, unbound IRGPP) and the anisotropy intensity increases. Subsequently, a compound which can dissociate the interaction of the IRGPP-partner complex, will result in a decrease in anisotropy (i.e., the labeled IRGPP rotates more rapidly), which indicates the compound has interfered with the binding of IRGPP to its binding partner.

A more traditional assay would involve labeling the IRGPP binding partner with an isotope such as ¹²⁵I, incubating with the IRGPP, then immunoprecipitating of the IRGPP. Compounds that increase the free IRGPP will decrease the precipitated counts. To avoid using radioactivity, the IRGPP binding partner could be labeled with an enzyme-conjugated antibody instead.

Alternatively, the IRGPP binding partner could be immobilized on the surface of an assay plate and the IRGPP could be labeled with a radioactive tag. A rise in the number of counts would identify compounds that had interfered with binding of the IRGPP and its binding partner.

Evaluation of binding interactions may further be performed using Biacore technology, wherein the IRGPP or its binding partner is bound to a micro chip, either directly by chemical modification or tethered via antibody-epitope association (e.g., antibody to the IRGPP), antibody directed to an epitope tag (e.g., His tagged) or fusion protein (e.g., GST). A second protein or proteins is/are then applied via flow over the “chip” and the change in signal is detected. Finally, test compounds are applied via flow over the “chip” and the change in signal is detected.

Once a series of potential compounds has been identified for a combination of IRGPP and IRGPP binding partner, a bioassay can be used to select the most promising candidates. For example, a cellular assay that measures cell proliferation in presence of the IRGPP and the IRGPP binding partner was described above. This assay could be modified to test the effectiveness of small molecules that interfere with binding of an IRGPP and its binding partner in enhancing cellular proliferation. An increase in cell proliferation would correlate with a compound's potency.

The test compounds of the present invention are generally either small molecules or biomolecules. Small molecules include, but are not limited to, inorganic molecules and small organic molecules. Biomolecules include, but are not limited to, naturally-occurring and synthetic compounds that have a bioactivity in mammals, such as lipids, steroids, polypeptides, polysaccharides, and polynucleotides. In one preferred embodiment, the test compound is a small molecule. In another preferred embodiment, the test compound is a biomolecule. One skilled in the art will appreciate that the nature of the test compound may vary depending on the nature of the IRGPP. For example, if the IRGPP is an orphan receptor having an unknown ligand, the test compound may be any of a number of biomolecules which may act as cognate ligand, including but not limited to, cytokines, lipid-derived mediators, small biogenic amines, hormones, neuropeptides, or proteases.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds. As used herein, the term “binding partner” refers to a molecule which serves as either a substrate for an IRGPP, or alternatively, as a ligand having binding affinity to the IRGPP.

### High-Throughput Screening Assays

The invention provides methods of conducting high-throughput screening for test compounds capable of inhibiting activity or expression of an IRGPP of the present invention.

In one embodiment, the method of high-throughput screening involves combining test compounds and the IRGPP and detecting the effect of the test compound on the IRGPP.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques are well-known in the art and are capable of high-throughput and ultra high throughput screening for activity, including but not limited to BRET® or FRET® (both by Packard Instrument Co., Meriden, Conn.). The ability to screen a large volume and a variety of test compounds with great sensitivity permits analysis of the therapeutic targets of the invention to further provide potential inhibitors of influenza. For example, where the IRG encodes an orphan receptor with an unidentified ligand, high-throughput assays may be utilized to identify the ligand, and to further identify test compounds which prevent binding of the receptor to the ligand. The BIACORE® system may also be manipulated to detect binding of test compounds with individual components of the therapeutic target, to detect binding to either the encoded protein or to the ligand.

By combining test compounds with IRGPPs of the invention and determining the binding activity between them, diagnostic analysis can be performed to elucidate the coupling systems. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. Preferably, the high-throughput screening assay of the present invention utilizes label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit activity of a protein encoded by the IRGs listed in Table 3, by combining the test compounds and the protein in high-throughput assays such as BIACORE®, or in fluorescence-based assays such as BRET®. In addition, high-throughput assays may be utilized to identify specific factors which bind to the encoded proteins, or alternatively, to identify test compounds which prevent binding of the receptor to the binding partner. In the case of orphan receptors, the binding partner may be the natural ligand for the receptor. Moreover, the high-throughput screening assays may be modified to determine whether test compounds can bind to either the encoded protein or to the binding partner (e.g., substrate or ligand) which binds to the protein.

### Detection Methods

Detection and measurement of the relative amount of an IRG product (polynucleotide or polypeptide) of the invention can be by any method known in the art. Typical methodologies for detection of a transcribed polynucleotide include RNA extraction from a cell or tissue sample, followed by hybridization of a labeled probe (i.e., a complementary polynucleotide molecule) specific for the target RNA to the extracted RNA and detection of the probe (i.e., Northern blotting).

Typical methodologies for peptide detection include protein extraction from a cell or tissue sample, followed by binding of an antibody specific for the target protein to the protein sample, and detection of the antibody. For example, detection of desmin may be accomplished using polyclonal antibody anti-desmin. Antibodies are generally detected by the use of a labeled secondary antibody. The label can be a radioisotope, a fluorescent compound, an enzyme, an enzyme co-factor, or ligand. Such methods are well understood in the art.

Detection of specific polynucleotide molecules may also be assessed by gel electrophoresis, column chromatography, or direct sequencing, quantitative PCR (in the case of polynucleotide molecules), RT-PCR, or nested-PCR among many other techniques well known to those skilled in the art.

Detection of the presence or number of copies of all or a part of an IRG of the invention may be performed using any method known in the art. Typically, it is convenient to assess the presence and/or quantity of a DNA or cDNA by Southern analysis, in which total DNA from a cell or tissue sample is extracted and hybridized with a labeled probe (i.e., a complementary DNA molecules). The probe is then detected and quantified. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Detection of specific polypeptide molecules may be assessed by gel electrophoresis, Western blot, column chromatography, or direct sequencing, among many other techniques well known to those skilled in the art.

An exemplary method for detecting the presence or absence of an IRGPP or IRGPN in a biological sample involves contacting a biological sample with a compound or an agent capable of detecting the IRGPP or IRGPN (e.g., mRNA, genomic DNA). A preferred agent for detecting mRNA or genomic DNA corresponding to an IRG or IRGPP of the invention is a labeled polynucleotide probe capable of hybridizing to a mRNA or genomic DNA of the invention. In a most preferred embodiment, the polynucleotides to be screened are arranged on a GeneChip®. Suitable probes for use in the diagnostic assays of the invention are described herein.

A preferred agent for detecting an IRGPP is an antibody capable of binding to the IRGPP, preferably an antibody with a detectable label. Antibodies can be polyclonal or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect IRG mRNA, protein or genomic DNA in a biological sample in vitro as well as in vivo. For example, in vitro techniques for detection of IRG mRNA include Northern hybridizations and in situ hybridizations. In vitro techniques for detection of IRGPP include enzyme linked immunosorbent assays (ELISAs), Western blots, immunoprecipitations and immunofluorescence. In vitro techniques for detection of IRG genomic DNA include Southern hybridizations. Furthermore, in vivo techniques for detection of IRGPP include introducing into a subject a labeled anti-IRGPP antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a tissue or serum sample isolated by conventional means from a subject, e.g., a biopsy or blood draw.

### Detection of Genetic Alterations

The methods of the invention can also be used to detect genetic alterations in an IRG, thereby determining if a subject with the altered gene is at risk for damage characterized by aberrant regulation in IRG expression or activity. In preferred embodiments, the methods include detecting, in a sample of cells from the subject, the presence or absence of a genetic alteration characterized by at least one alteration affecting the integrity of an IRG, or the aberrant expression of the IRG. For example, such genetic alterations can be detected by ascertaining the existence of at least one of the following: 1) deletion of one or more nucleotides from an IRG; 2) addition of one or more nucleotides to an IRG; 3) substitution of one or more nucleotides of an IRG, 4) a chromosomal rearrangement of an IRG; 5) alteration in the level of a messenger RNA transcript of an IRG, 6) aberrant modification of an IRG, such as of the methylation pattern of the genomic DNA, 7) the presence of a non-wild type splicing pattern of a messenger RNA transcript of an IRG, 8) non-wild type level of an IRGPP, 9) allelic loss of an IRG, and 10) inappropriate post-translational modification of an IRGPP. As described herein, there are a large number of assays known in the art, which can be used for detecting alterations in an IRG or an IRG product. A preferred biological sample is a blood sample isolated by conventional means from a subject.

In certain embodiments, detection of the alteration involves the use of a probe/primer in a polymerase chain reaction (PCR), such as anchor PCR or RACE PCR, or, alternatively, in a ligation chain reaction (LCR), the latter of which can be particularly useful for detecting point mutations in the IRG. This method can include the steps of collecting a sample of cells from a subject, isolating a polynucleotide sample (e.g., genomic, mRNA or both) from the cells of the sample, contacting the polynucleotide sample with one or more primers which specifically hybridize to an IRG under conditions such that hybridization and amplification of the IRG (if present) occurs, and detecting the presence or absence of an amplification product, or detecting the size of the amplification product and comparing the length to a control sample. It is understood that PCR and/or LCR may be desirable to be used as a preliminary amplification step in conjunction with any of the techniques used for detecting mutations described herein.

Alternative amplification methods include: self-sustained sequence replication, transcriptional amplification system, Q-Beta Replicase, or any other polynucleotide amplification method, followed by the detection of the amplified molecules using techniques well known to those of skill in the art. These detection schemes are especially useful for the detection of polynucleotide molecules if such molecules are present in very low numbers.

In an alternative embodiment, mutations in an IRG from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicate mutations in the sample DNA. Moreover, sequence specific ribozymes (see, for example, U.S. Pat. No. 5,498,531) can be used to score for the presence of specific mutations by development or loss of a ribozyme cleavage site.

In other embodiments, genetic mutations in an IRG can be identified by hybridizing sample and control polynucleotides, e.g., DNA or RNA, to high density arrays containing hundreds or thousands of oligonucleotides probes. For example, genetic mutations in an IRG can be identified in two dimensional arrays containing light generated DNA probes. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the IRG and detect mutations by comparing the sequence of the sample IRG with the corresponding wild-type (control) sequence. It is also contemplated that any of a variety of automated sequencing procedures can be utilized when performing the diagnostic assays, including sequencing by mass spectrometry.

Other methods for detecting mutations in an IRG include methods in which protection from cleavage agents is used to detect mismatched bases in RNA/RNA or RNA/DNA heteroduplexes. In general, the art technique of “mismatch cleavage” starts by providing heteroduplexes by hybridizing (labeled) RNA or DNA containing the wild-type IRG sequence with potentially mutant RNA or DNA obtained from a tissue sample. The double-stranded duplexes are treated with an agent which cleaves single-stranded regions of the duplex, which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in IRG cDNAs obtained from samples of cells. For example, the mutY enzyme of E. coli cleaves A at G/A mismatches and the thymidine DNA glycosylase from HeLa cells cleaves T at G/T mismatches. According to an exemplary embodiment, a probe based on an IRG sequence, e.g., a wild-type IRG sequence, is hybridized to cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in IRGs. For example, single-strand conformation polymorphism (SSCP) may be used to detect differences in electrophoretic mobility between mutant and wild type polynucleotides. Single-stranded DNA fragments of sample and control IRG polynucleotides will be denatured and allowed to renature. The secondary structure of single-stranded polynucleotides varies according to sequence. The resulting alteration in electrophoretic mobility enables the detection of even a single base change. The DNA fragments may be labeled or detected with labeled probes. The sensitivity of the assay may be enhanced by using RNA (rather than DNA) in which the secondary structure is more sensitive to a change in sequence. In a preferred embodiment, the subject method utilizes heteroduplex analysis to separate double-stranded heteroduplex molecules on the basis of changes in electrophoretic mobility (Keen et al. Trends Genet. 7:5, 1991).

In yet another embodiment the movement of mutant or wild-type fragments in polyacrylamide gels containing a gradient of denaturant is assayed using denaturing gradient gel electrophoresis (DGGE). When DGGE is used as the method of analysis, DNA will be modified to insure that it does not completely denature, for example, by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner Biophys Chem 265:12753, 1987).

Examples of other techniques for detecting point mutations include, but are not limited to, selective oligonucleotide hybridization, selective amplification, and selective primer extension. For example, oligonucleotide primers may be prepared in which the known mutation is placed centrally and then hybridized to target DNA under conditions which permit hybridization only if a perfect match is found (Saiki et al. Proc. Natl. Acad. Sci. USA 86:6230, 1989). Such allele specific oligonucleotides are hybridized to PCR amplified target or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) or at the extreme 3′ end of one primer where, under appropriate conditions, mismatch can prevent or reduce polymerase extension. In addition, it may be desirable to introduce a novel restriction site in the region of the mutation to create cleavage-based detection. It is anticipated that, in certain embodiments, amplification may also be performed using Taq ligase for amplification. In such cases, ligation will occur only if there is a perfect match at the 3′ end of the 5′ sequence, thus making it possible to detect the presence of a known mutation at a specific site by looking for the presence or absence of amplification.

### Monitoring Effects During Clinical Trials

Monitoring the influence of agents (e.g., drugs, small molecules, proteins, nucleotides) on the expression of an IRG or activity of an IRGPP can be applied not only in basic drug screening, but also in clinical trials. For example, the effectiveness of an agent determined by a screening assay, as described herein to decrease an IRGPP activity, can be monitored in clinical trials of subjects exhibiting increased IRGPP activity. In such clinical trials, the activity of the IRGPP can be used as a “read-out” of the phenotype of a particular tissue.

For example, and not by way of limitation, IRGs that are modulated in tissues by treatment with an agent can be identified. Thus, to study the effect of agents on the IRGPP in a clinical trial, cells can be isolated and RNA prepared and analyzed for the levels of expression of an IRG. The levels of gene expression or a gene expression pattern can be quantified by Northern blot analysis, RT-PCR or GeneChip® as described herein, or alternatively by measuring the amount of protein produced, by one of the methods as described herein, or by measuring the levels of activity of IRGPP. In this way, the gene expression pattern can serve as a read-out, indicative of the physiological response of the cells to the agent. Accordingly, this response state may be determined before treatment and at various points during treatment of the individual with the agent.

In a preferred embodiment, the present invention provides a method for monitoring the effectiveness of treatment of a subject with an agent (e.g., an agonist, antagonist, peptidomimetic, protein, peptide, polynucleotide, small molecule, or other drug candidate identified by the screening assays described herein) including the steps of (i) obtaining a pre-administration sample from a subject prior to administration of the agent; (ii) detecting the level of expression of an IRG protein or mRNA in the pre-administration sample; (iii) obtaining one or more post-administration samples from the subject; (iv) detecting the level of expression or activity of the IRG protein or mRNA in the post-administration samples; (v) comparing the level of expression or activity of the IRG protein or mRNA in the pre-administration sample with the IRG protein or mRNA the post administration sample or samples; and (vi) altering the administration of the agent to the subject accordingly. According to such an embodiment, IRG expression or activity may be used as an indicator of the effectiveness of an agent, even in the absence of an observable phenotypic response.

### Methods of Treatment

The present invention provides for both prophylactic and therapeutic methods of treating a subject at risk for, susceptible to or diagnosed with influenza.

In one aspect, the invention provides a method for preventing influenza in a subject by administering to the subject an IRG product or an agent which modulates IRG protein expression or activity.

Administration of a prophylactic agent can occur prior to the manifestation of symptoms characteristic of the differential IRG protein expression, such that influenza is prevented or, alternatively, delayed in its progression. Depending on the type of IRG aberrancy (e.g., typically a modulation outside the normal standard deviation), for example, an IRG product, IRG agonist or antagonist agent can be used for treating the subject. The appropriate agent can be determined based on screening assays described herein.

Another aspect of the invention pertains to methods of modulating IRG protein expression or activity for therapeutic purposes. Accordingly, in an exemplary embodiment, the modulatory method of the invention involves contacting a cell with an agent that modulates one or more of the activities of a IRG product activity associated with the cell. An agent that modulates IRG product activity can be an agent as described herein, such as a polynucleotide (e.g., an antisense molecule) or a polypeptide (e.g., a dominant-negative mutant of an IRGPP), a naturally-occurring target molecule of an IRGPP (e.g., an IRGPP substrate), an anti-IRGPP antibody, an IRG modulator (e.g., agonist or antagonist), a peptidomimetic of an IRG protein agonist or antagonist, or other small molecules.

The invention further provides methods of modulating a level of expression of an IRG of the invention, comprising administration to a subject having influenza, a variety of compositions which correspond to the IRGs of Table 3, including proteins or antisense oligonucleotides. The protein may be provided by further providing a vector comprising a polynucleotide encoding the protein to the cells. Alternatively, the expression levels of the IRGs of the invention may be modulated by providing an antibody, a plurality of antibodies or an antibody conjugated to a therapeutic moiety.

### Determining Efficacy of a Test Compound or Therapy

The invention also provides methods of assessing the efficacy of a test compound or therapy for inhibiting influenza in a subject. These methods involve isolating samples from a subject suffering from influenza, who is undergoing treatment or therapy, and detecting the presence, quantity, and/or activity of one or more IRGs of the invention in the first sample relative to a second sample. Where the efficacy of a test compound is determined, the first and second samples are preferably sub-portions of a single sample taken from the subject, wherein the first portion is exposed to the test compound and the second portion is not. In one aspect of this embodiment, the IRG is expressed at a substantially decreased level in the first sample, relative to the second. Most preferably, the level of expression in the first sample approximates (i.e., is less than the standard deviation for normal samples) the level of expression in a third control sample, taken from a control sample of normal tissue. This result suggests that the test compound inhibits the expression of the IRG in the sample. In another aspect of this embodiment, the IRG is expressed at a substantially increased level in the first sample, relative to the second. Most preferably, the level of expression in the first sample approximates (i.e., is less than the standard deviation for normal samples) the level of expression in a third control sample, taken from a control sample of normal tissue. This result suggests that the test compound augments the expression of the IRG in the sample.

Where the efficacy of a therapy is being assessed, the first sample obtained from the subject is preferably obtained prior to provision of at least a portion of the therapy, whereas the second sample is obtained following provision of the portion of the therapy. The levels of IRG product in the samples are compared, preferably against a third control sample as well, and correlated with the presence, or risk of presence, of influenza. Most preferably, the level of IRG product in the second sample approximates the level of expression of a third control sample. In the present invention, a substantially decreased level of expression of an IRG indicates that the therapy is efficacious for treating influenza.

### Pharmaceutical Compositions

The invention is further directed to pharmaceutical compositions comprising the test compound, or bioactive agent, or an IRG modulator (i.e., agonist or antagonist), which may further include an IRG product, and can be formulated as described herein. Alternatively, these compositions may include an antibody which specifically binds to an IRG protein of the invention and/or an antisense polynucleotide molecule which is complementary to an IRGPN of the invention and can be formulated as described herein.

One or more of the IRGs of the invention, fragments of IRGs, IRG products, fragments of IRG products, IRG modulators, or anti-IRGPP antibodies of the invention can be incorporated into pharmaceutical compositions suitable for administration.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a polypeptide or polynucleotide corresponding to an IRG of the invention. Such methods comprise formulating a pharmaceutically acceptable carrier with an agent which modulates expression or activity of an IRG. Such compositions can further include additional active agents. Thus, the invention further includes methods for preparing a pharmaceutical composition by formulating a pharmaceutically acceptable carrier with an agent which modulates expression or activity of an IRG and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), intraperitoneal, transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an IRGPP or an anti-IRGPP antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form, as used herein, includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The IRGs of the invention can be inserted into gene delivery vectors and used as gene therapy vectors. Gene therapy vectors can be delivered to a subject by, for example, intravenous administration, intraportal administration, intrabiliary administration, intra-arterial administration, direct injection into the liver parenchyma, by intramusclular injection, by inhalation, by perfusion, or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Kits

The invention also encompasses kits for detecting the presence of an IRG product in a biological sample, the kit comprising reagents for assessing expression of the IRGs of the invention. Preferably, the reagents may be an antibody or fragment thereof, wherein the antibody or fragment thereof specifically binds with a protein corresponding to an IRG from Table 3. For example, antibodies of interest may be prepared by methods known in the art. Optionally, the kits may comprise a polynucleotide probe wherein the probe specifically binds with a transcribed polynucleotide corresponding to an IRG selected from the group consisting of the IRGs listed in Table 3. The kits may also include an array of IRGs arranged on a biochip, such as, for example, a GeneChip®. The kit may contain means for determining the amount of the IRG protein or mRNA in the sample; and means for comparing the amount of the IRG protein or mRNA in the sample with a control or standard. The compound or agent can be packaged in a suitable container. The kit can further comprise instructions for using the kit to detect IRG protein or polynucleotide.

The invention further provides kits for assessing the suitability of each of a plurality of compounds for inhibiting influenza in a subject. Such kits include a plurality of compounds to be tested, and a reagent (i.e., antibody specific to corresponding proteins, or a probe or primer specific to corresponding polynucleotides) for assessing expression of an IRG listed in Table 3.

### Arrays and Biochips

The invention also includes an array comprising a panel of IRGs of the present invention. The array can be used to assay expression of one or more genes in the array.

It will be appreciated by one skilled in the art that the panels of IRGs of the invention may conveniently be provided on solid supports, such as a biochip. For example, polynucleotides may be coupled to an array (e.g., a biochip using GeneChip® for hybridization analysis), to a resin (e.g., a resin which can be packed into a column for column chromatography), or a matrix (e.g., a nitrocellulose matrix for Northern blot analysis). The immobilization of molecules complementary to the IRG(s), either covalently or noncovalently, permits a discrete analysis of the presence or activity of each IRG in a sample. In an array, for example, polynucleotides complementary to each member of a panel of IRGs may individually be attached to different, known locations on the array. The array may be hybridized with, for example, polynucleotides extracted from a blood or colon sample from a subject. The hybridization of polynucleotides from the sample with the array at any location on the array can be detected, and thus the presence or quantity of the IRG and IRG transcripts in the sample can be ascertained. In a preferred embodiment, an array based on a biochip is employed. Similarly, Western analyses may be performed on immobilized antibodies specific for IRGPPs hybridized to a protein sample from a subject.

It will also be apparent to one skilled in the art that the entire IRG product (protein or polynucleotide) molecule need not be conjugated to the biochip support; a portion of the IRG product or sufficient length for detection purposes (i.e., for hybridization), for example a portion of the IRG product which is 7, 10, 15, 20, 25, 30, 35, 40, 45, 50, 55, 60, 65, 70, 75, 100 or more nucleotides or amino acids in length may be sufficient for detection purposes.

In addition to such qualitative determination, the invention allows the quantitation of gene expression in the biochip. Thus, not only tissue specificity, but also the level of expression of a battery of IRGs in the tissue is ascertainable. Thus, IRGs can be grouped on the basis of their tissue expression per se and level of expression in that tissue. As used herein, a “normal level of expression” refers to the level of expression of a gene provided in a control sample, typically the control is taken from either a non-diseased animal or from a subject who has not suffered from influenza. The determination of normal levels of expression is useful, for example, in ascertaining the relationship of gene expression between or among tissues. Thus, one tissue or cell type can be perturbed and the effect on gene expression in a second tissue or cell type can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more genes in the array. This can occur in various biological contexts, as disclosed herein, for example development and differentiation, disease progression, in vitro processes, such as cellular transformation and activation.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

Importantly, the invention provides arrays useful for ascertaining differential expression patterns of one or more genes identified in diseased tissue versus non-diseased tissue. This provides a battery of genes that serve as a molecular target for diagnosis or therapeutic intervention. In particular, biochips can be made comprising arrays not only of the IRGs listed in Table 3, but of IRGs specific to subjects suffering from specific manifestations or stages of the disease.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups. Linkers, such as homo- or hetero-bifunctional linkers, may also be used.

In an embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques.

Modifications to the above-described compositions and methods of the invention, according to standard techniques, will be readily apparent to one skilled in the art and are meant to be encompassed by the invention. This invention is further illustrated by the following examples which should not be construed as limiting. The contents of all references, patents and published patent applications cited throughout this application, as well as the Figures and Tables are incorporated herein by reference.

### Host Cells

Another aspect of the invention pertains to host cells into which a polynucleotide molecule of the invention, e.g., an IRG of Table 3 or homolog thereof, is introduced within an expression vector, a gene delivery vector, or a polynucleotide molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an IRG can be expressed in bacterial cells such as E. coli, insect cells, yeast or mammalian cells (such as Chinese hamster ovary cells (CHO), COS cells, Fischer 344 rat cells, HLA-B27 rat cells, HeLa cells, A549 cells, or 293 cells. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotide (e.g., DNA) into a host cell, including calcium phosphate or calcium chloride co-precipitation, DAKD-dextran-mediated transfection, lipofection, or electoporation.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotide encoding a selectable flag can be introduced into a host cell on the same vector as that encoding STK3P23 or can be introduced on a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) an IRG product. Accordingly, the invention further provides methods for producing an IRG product using the host cells of the invention. In one embodiment, the method comprises culturing the host cell of invention (into which a recombinant expression vector encoding an IRG has been introduced) in a suitable medium such that an IRG product is produced. In another embodiment, the method further comprises isolating the IRG product from the medium or the host cell.

### Transgenic and Knockout Animals

The host cells of the invention can also be used to produce non-human transgenic animals. For example, in one embodiment, a host cell of the invention is a fertilized oocyte or an embryonic stem cell into which an IRG sequence has been introduced. Such host cells can then be used to create non-human transgenic animals in which an exogenous sequence encoding an IRG has been introduced into their genome or homologous recombinant animals in which an endogenous sequence encoding an IRG has been altered. Such animals are useful for studying the function and/or activity of the IRG and for identifying and/or evaluating modulators of the IRG activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous IRG has been altered by homologous recombination between the endogenous gene and an exogenous DNA molecule introduced into a cell of the animal, e.g., an embryonic cell of the animal, prior to development of the animal.

A transgenic animal of the invention can be created by introducing an IRG-encoding polynucleotide into the mate pronuclei of a fertilized oocyte, e.g., by microinjection or retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of an IRG to particular cells. Methods for generating transgenic animals via embryo manipulation and microinjection, particularly animals such as mice, have become conventional in the art. Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying an IRG can further be bred to other transgenic animals carrying other transgenes.

To create a homologous recombinant animal (knockout animal), a vector is prepared which contains at least a portion of a gene of the invention into which a deletion, addition or substitution has been introduced to thereby alter, e.g., functionally disrupt, the gene. The gene can be a human gene, but more preferably, is a non-human homolog of a human gene of the invention (e.g., a homolog of an IRG). For example, a mouse gene can be used to construct a homologous recombination polynucleotide molecule, e.g., a vector, suitable for altering an endogenous gene of the invention in the mouse genome. In a preferred embodiment, the homologous recombination polynucleotide molecule is designed such that, upon homologous recombination, the endogenous gene of the invention is functionally disrupted (i.e., no longer encodes a functional protein; also referred to as a “knockout” vector). Alternatively, the homologous recombination polynucleotide molecule can be designed such that, upon homologous recombination, the endogenous gene is mutated or otherwise altered but still encodes functional protein (e.g., the upstream regulatory region can be altered to thereby alter the expression of the endogenous IRG). In the homologous recombination polynucleotide molecule, the altered portion of the gene of the invention is flanked at its 5′ and 3′ ends by additional polynucleotide sequence of the gene of the invention to allow for homologous recombination to occur between the exogenous gene carried by the homologous recombination polynucleotide molecule and an endogenous gene in a cell, e.g., an embryonic stem cell. The additional flanking polynucleotide sequence is of sufficient length for successful homologous recombination with the endogenous gene.

Typically, several kilobases of flanking DNA (both at the 5′ and 3′ ends) are included in the homologous recombination polynucleotide molecule (see, e.g., Thomas, K. R. and Capecchi, M. R. (1987) Cell 51:503 for a description of homologous recombination vectors). The homologous recombination polynucleotide molecule is introduced into a cell, e.g., an embryonic stem cell line (e.g., by electroporation) and cells in which the introduced gene has homologously recombined with the endogenous gene are selected. The selected cells can then be injected into a blastocyst of an animal (e.g., a mouse) to form aggregation chimeras (see e.g., Bradley, S A. in Teratocareirtomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination polynucleotide molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage Pl. For a description of the cre/loxP recombinase system, see, e.g., Laksa et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Modifications to the above-described compositions and methods of the invention, according to standard techniques, will be readily apparent to one skilled in the art and are meant to be encompassed by the invention. This invention is further illustrated by the following examples which should not be construed as limiting. The contents of all references, patents and published patent applications cited throughout this application, as well as the Figures and Tables are incorporated herein by reference.

### EXAMPLES

### Example 1

### Construction of RHKO Vectors and Screening of Influenza Resistant Clones

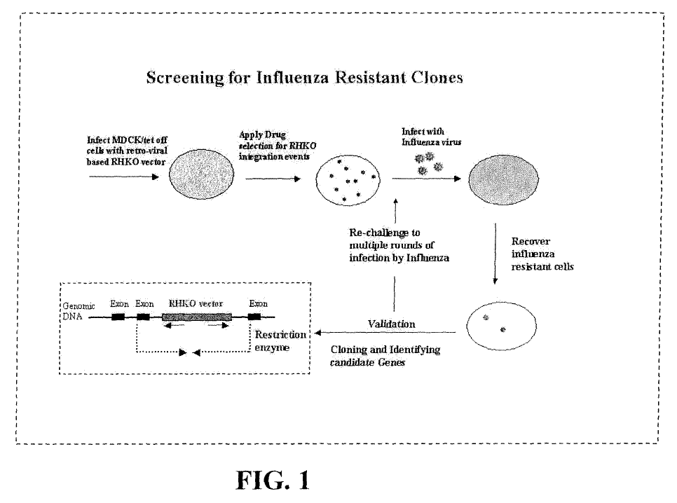

RHKO vectors were constructed as described by Li et al. (Li et al. Cell, 85: 319-329, 1996). The procedure for screening influenza resistant clones is depicted in FIG. 1. Briefly, Madin Darby Canine Kidney (MDCK) cells were infected with a retro-viral based random homozygous knock-out (RHKO) vector. Cells containing the stably integrated vector were selected and subjected to influenza infection using the MOI which would result in 100% killing of parental cells between 48 to 72 hour. The influenza resistant cells were expanded and subject to additional rounds of influenza infection with higher multiplicity of infection (MOI). The resistant clones that survived multiple rounds of influenza infection were recovered. The influenza resistant phenotype was validated by testing the clones' resistance to multiple strains of influenza virus and by correlation of the phenotype with RHKO integration. The RHKO integration sites in the resistant cells were then cloned and identified as described in Example 2.

### Example 2

### Identification of Influenza Resistant Genes

The RHKO integration sites in the resistant cells were cloned and the sequences flanking the RHKO integration site were determined. The affected genes were identified by aligning the flanking sequences at the integration site to the Genebank database.

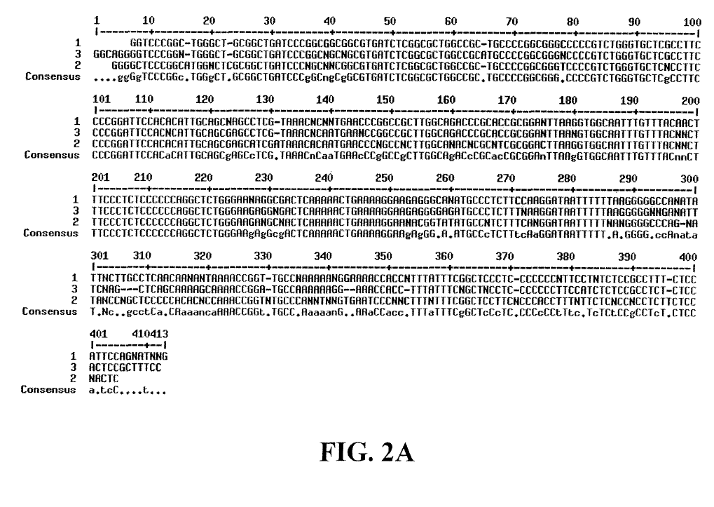

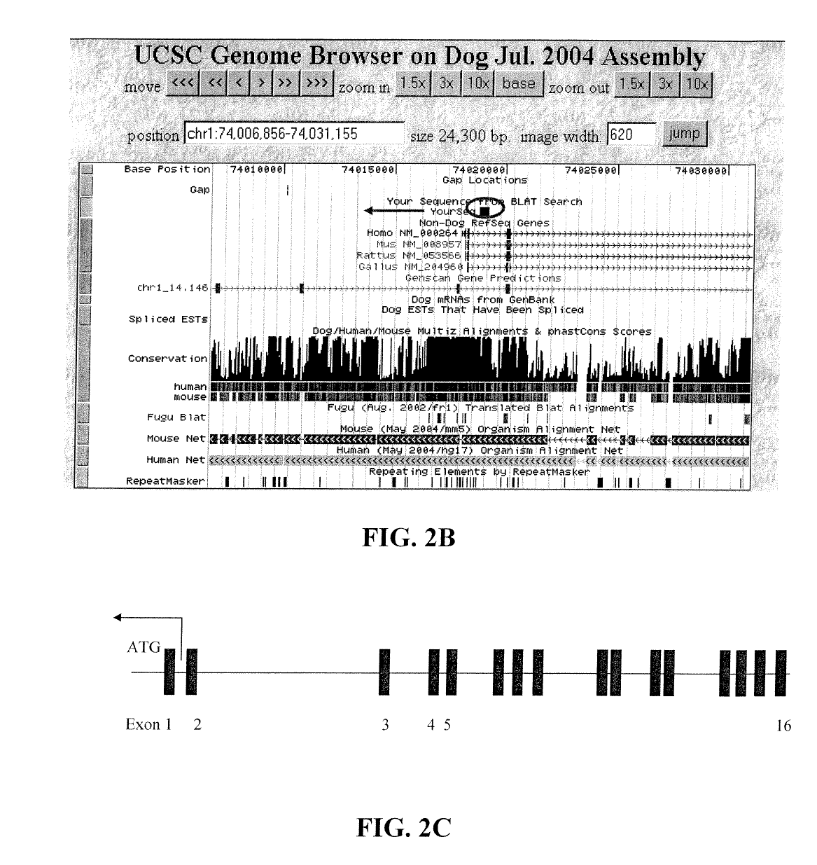

FIG. 2A shows the alignment of the 5′-end flanking sequences obtained from three subclones of influenza resistant clone 26-8-7. The consensus sequence derived from the alignment (SEQ ID NO:1) was used to identify the affected gene PTCH (SEQ ID NOS: 9 and 17). FIG. 2B depicts the genomic site of RHKO integration. As shown in FIG. 2C, the position of the RHKO integration indicate that the PTCH gene is likely to be inactivated by the antisense expression from the RHKO construct.

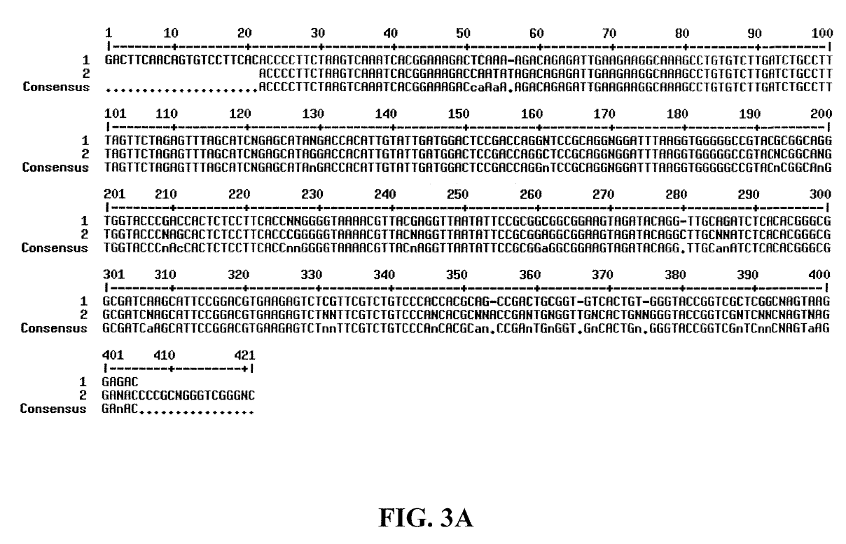

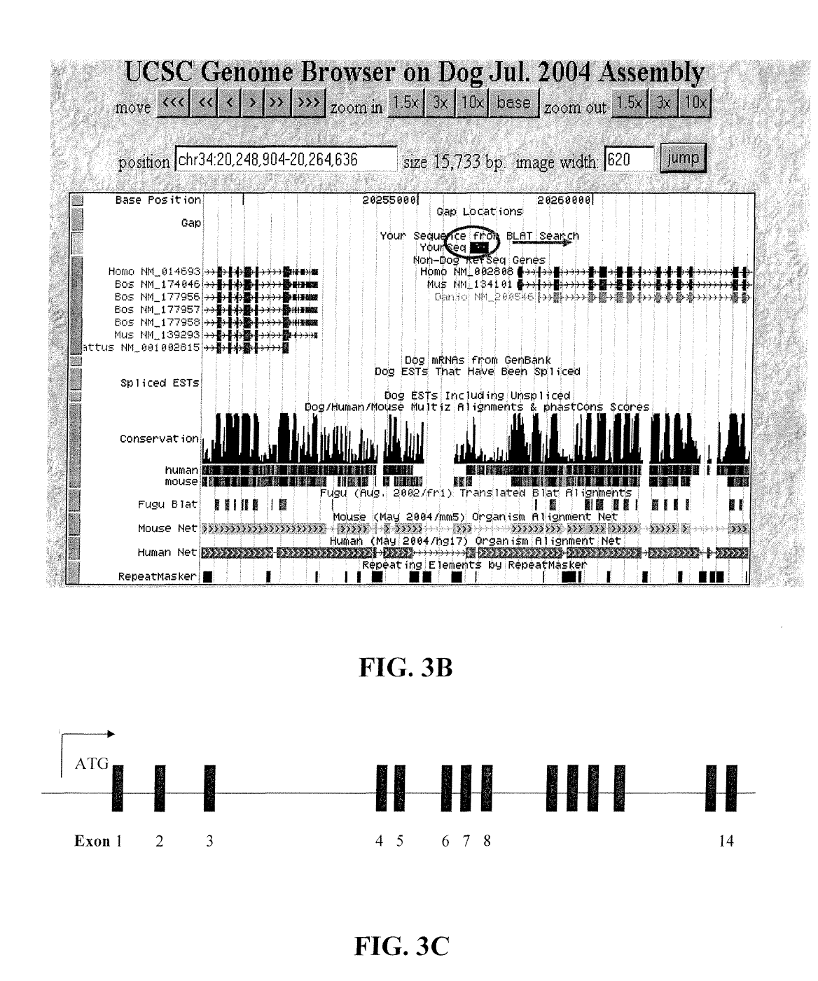

FIG. 3A shows the alignment of the 5′-end flanking sequences obtained from two subclones of influenza resistant clone R18-6. The consensus sequence derived from the alignment (SEQ ID NO:2) was used to identify the affected gene PSMD2 (SEQ ID NOS: 10 and 18). FIG. 3B depicts the genomic site of RHKO integration. As shown in FIG. 3C, the position of the RHKO integration indicate that the PSMD2 gene is likely to be overexpressed due to activation by the RHKO construct.

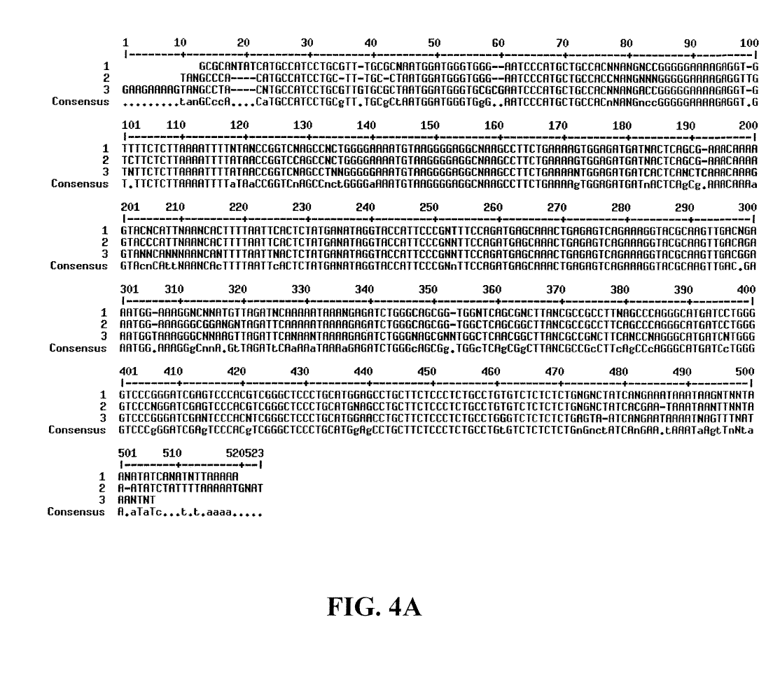

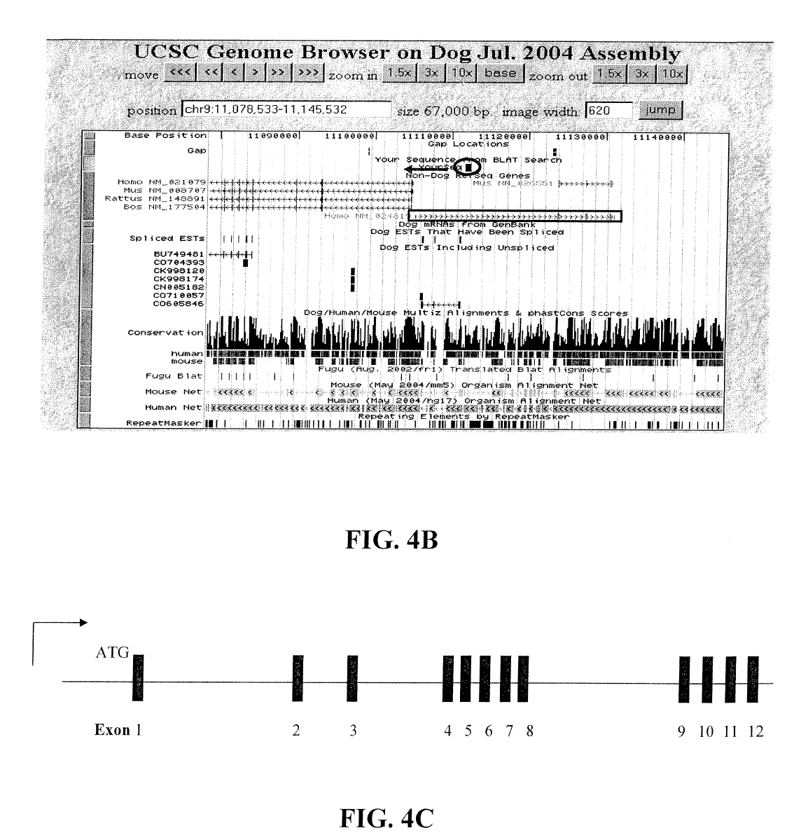

FIG. 4A shows the alignment of the 5′-end flanking sequences obtained from three subclones of influenza resistant clone R26-8-11. The consensus sequence derived from the alignment (SEQ ID NO:3) was used to identify the affected gene NMT1 (SEQ ID NOS: 11 and 19). FIG. 4B depicts the genomic site of RHKO integration. As shown in FIG. 4C, the position of the RHKO integration indicate that the NMT1 gene is likely to be inactivated by the disruption of promoter by the RHKO construct.

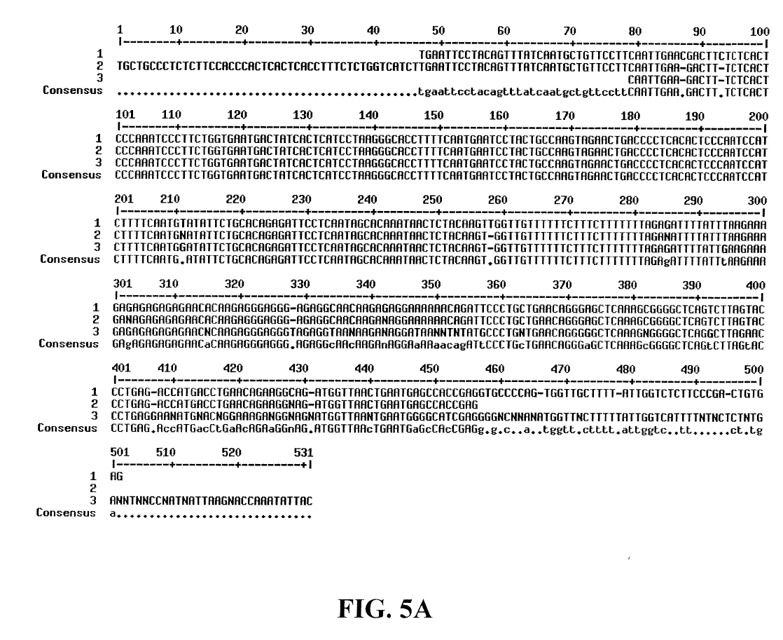

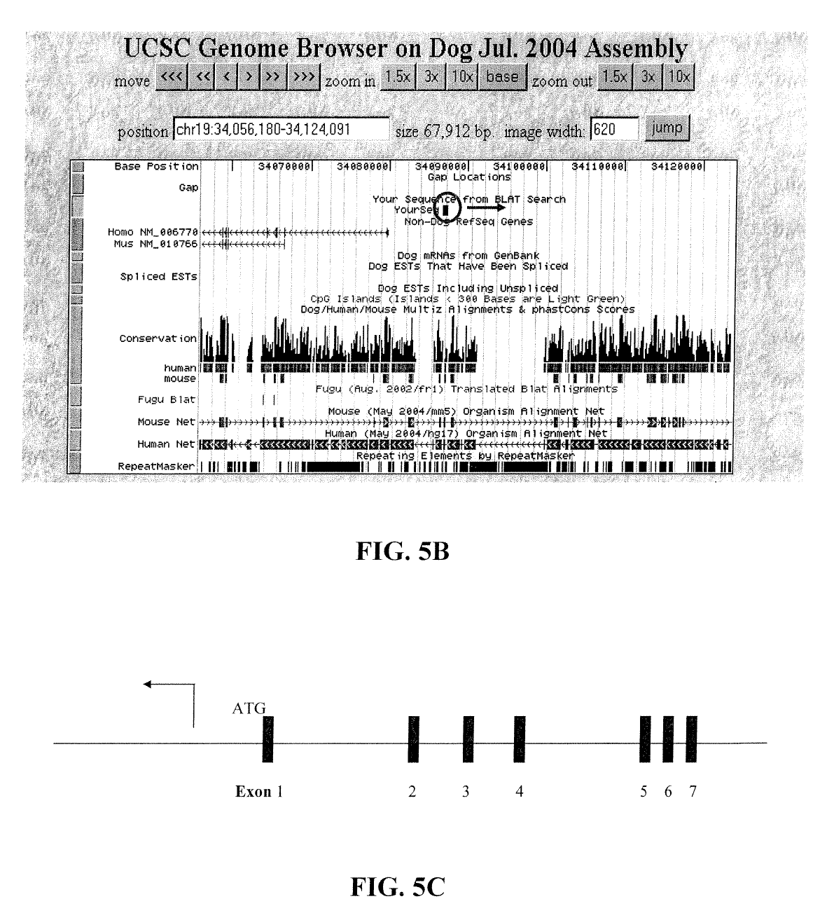

FIG. 5A shows the alignment of the 5′-end flanking sequences obtained from three subclones of influenza resistant clone 26-8-11. The consensus sequence derived from the alignment (SEQ ID NO:4) was used to identify the affected gene MACRO (SEQ ID NOS: 12 and 20). FIG. 5B depicts the genomic site of RHKO integration. As shown in FIG. 5C, the position of the RHKO integration indicate that the MACRO gene is likely to be overexpressed due to the integration of the RHKO construct.

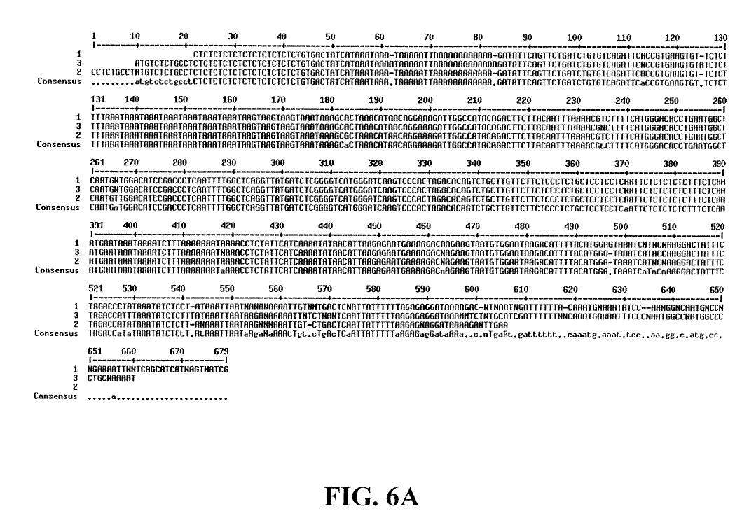

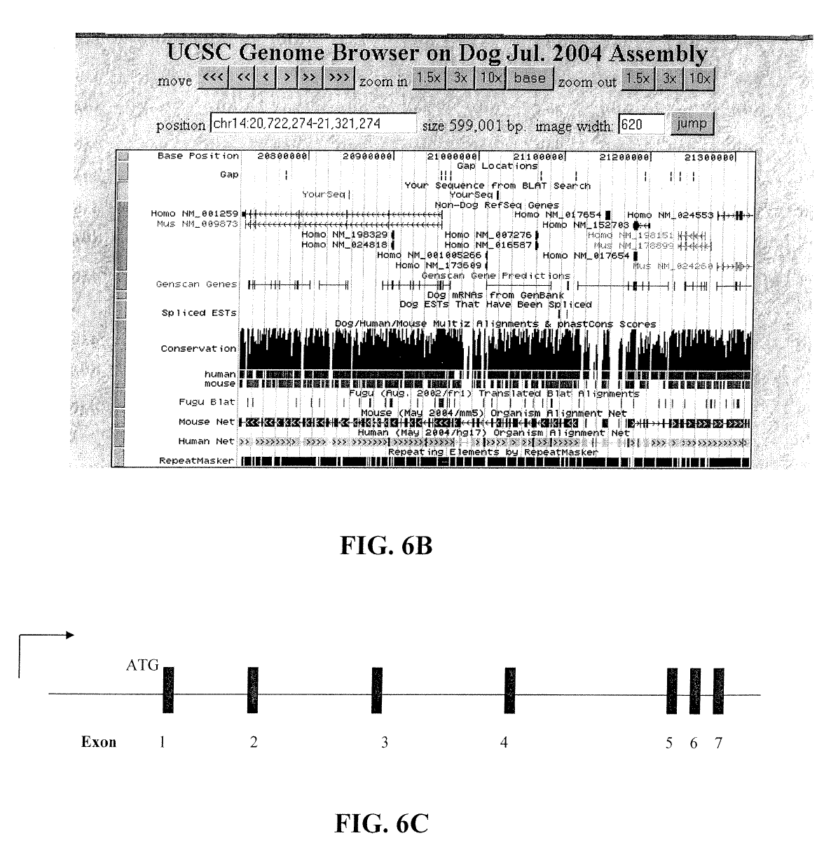

FIG. 6A shows the alignment of the 5′-end flanking sequences obtained from three subclones of influenza resistant clone R21-1. The consensus sequence derived from the alignment (SEQ ID NO:5) was used to identify the affected gene CDK6 (SEQ ID NOS: 13 and 21). FIG. 6B depicts the genomic site of RHKO integration. As shown in FIG. 6C, the position of the RHKO integration indicate that the CDK6 gene is likely to be inactivated by the integration of the RHKO construct due to the disruption of promoter.

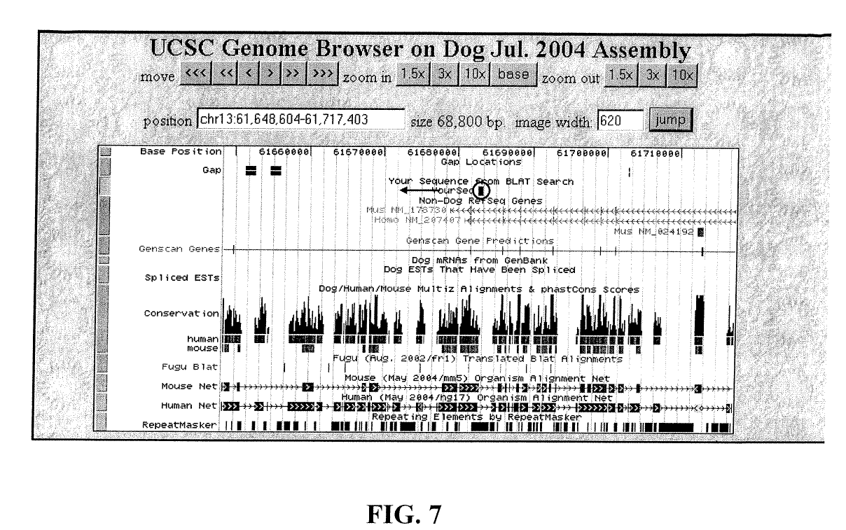

The 5′-end flanking sequence (SEQ ID NO: 6) obtained from influenza resistant clone R27-32 was used to identify the affected gene FLJ16046 (SEQ ID NOS: 14 and 22). FIG. 7 depicts the genomic site of RHKO integration. The position of the RHKO integration indicate that the FLJ1604 gene is likely to be overexpressed due to the integration of the RHKO construct.

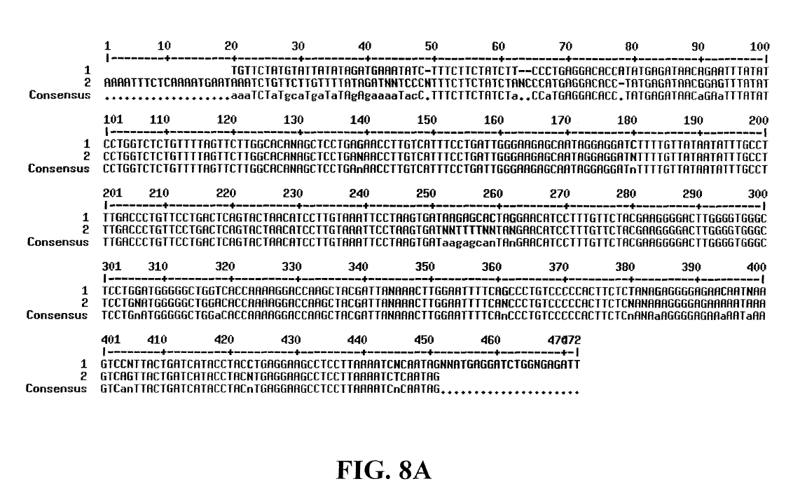

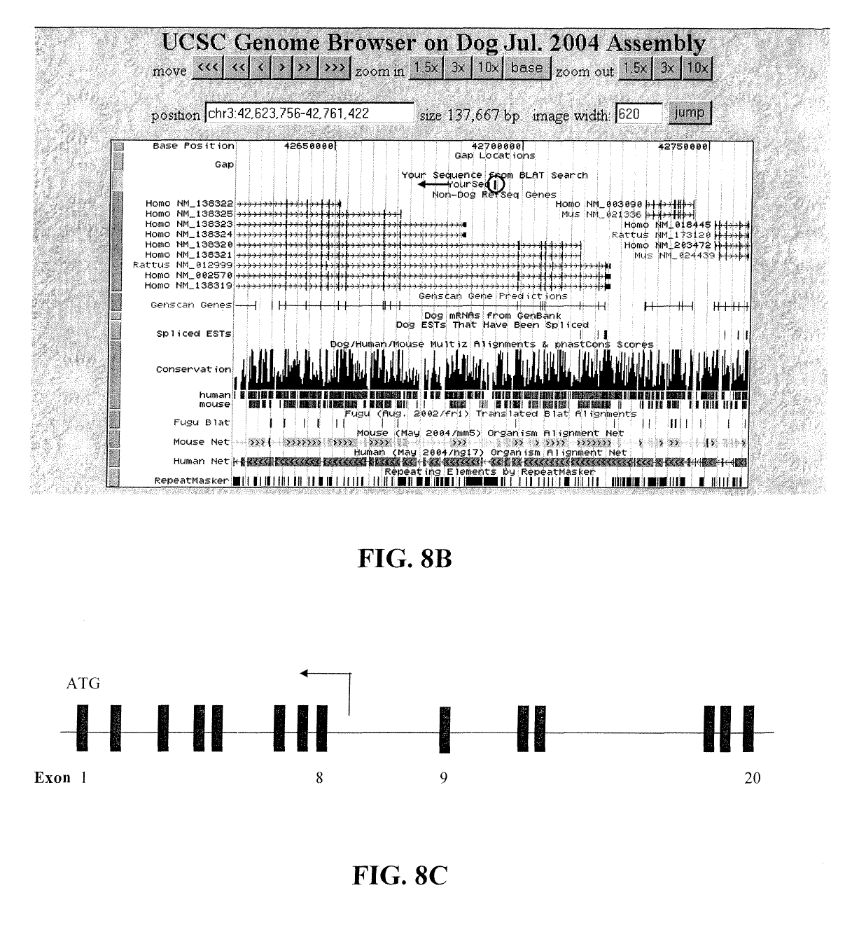

FIG. 8A shows the alignment of the 5′-end flanking sequences obtained from two subclones of influenza resistant clone R27-3-33. The consensus sequence derived from the alignment (SEQ ID NO:7) was used to identify the affected gene PCSK6 (SEQ ID NOS: 15 and 23). FIG. 8B depicts the genomic site of RHKO integration. As shown in FIG. 8C, the position of the RHKO integration indicate that the PCSK6 gene is likely to be inactivated by the antisense transcription from the RHKO construct.

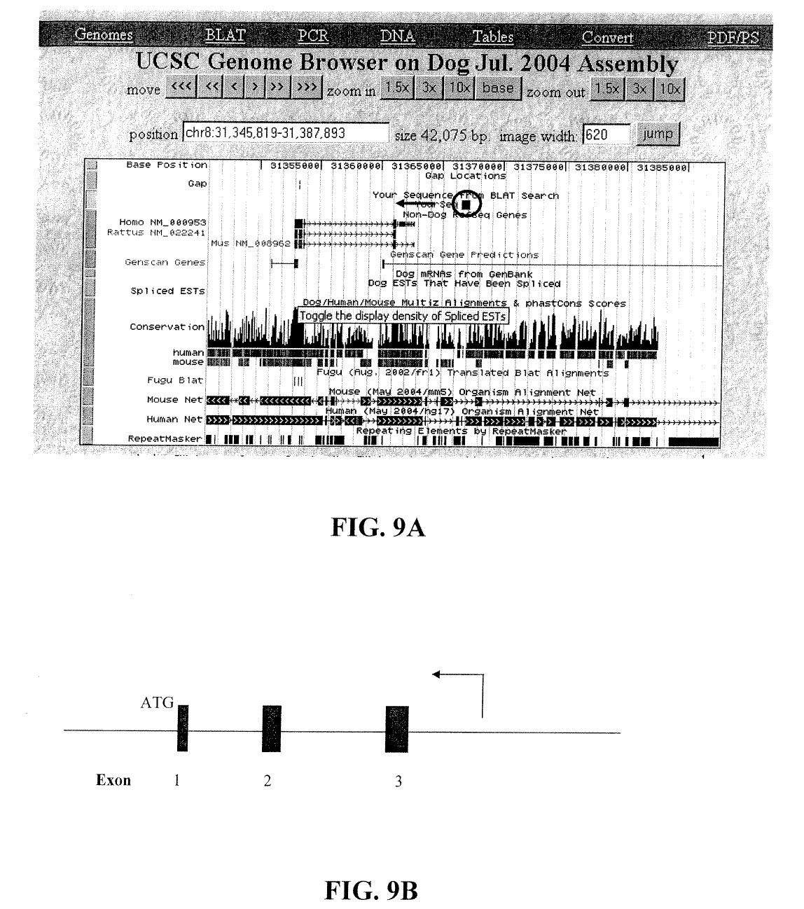

The 5′-end flanking sequence (SEQ ID NO: 8) obtained from influenza resistant clone R27-3-35 was used to identify the affected gene PTGDR (SEQ ID NOS: 16 and 24). FIG. 9A depicts the genomic site of RHKO integration. As shown in FIG. 9B, the position of the RHKO integration indicate that the PTGDR gene is likely to be inactivated by the antisense transcription from the RHKO construct.

PTCH flanking

SEQ ID NO: 1

TAAACGTAAAAAGTAGCCAAGCGCACGGGGGAAGGGCCCCGGCCGGCG
CAGGCAGGGGTCCCGGNTGGGCTGCGGCTGATCCCGGCNGCNGCGTGA
TCTCGGCGCTGGCCGCATGCCCCGGCGGGNCCCCGTCTGGGTGCTCGC
CTTCCCCGGATTCCACNCATTGCAGCGAGCCTCGTAAACNCAATGAAN
CCGGCCGCTTGGCAGACCCGCACCGCGGANTTAANGTGGCAATTTGTT
TACNNCTTTCCCTCTCCCCCCAGGCTCTGGGAAGAGGNGACTCAAAAA
CTGAAAAGGAAGAGGGGAGATGCCCTCTTTNAAGGATAATTTTTAAGG
GGGNNGANATTTCNAGCTCAGCAAAAGCAAAACCGGATGCCAAAAAAG
GAAACCACCTTTATTTCNGCTNCCTCCCCCCCTTCCATCTCTCCGCCT
CTCTCCACTCCGCTTTCCNCCCTCAAAAGATGTTAAAAAAATGTGGCA
GCATTTCNCGGGNNTTGGGACNGCAAANTAAGGNGCCAAGGGGCTANG
NCCATCTGGGGTTCTCCNNGGGCNCGGGTNTNCCGGGTCGNTGACCTC
GCGGACTGTNTGGCNNTCNTAGNATGGCNCCCGCANAANCGCTNTNCA
NTNNTCTGTNAAAAGGNATNNCTTTTAANCNTCCTTACNACCCNTCCN
ACCNCACCCAAATNANNTTTNTTCTTGNATATGCTGATNNATCNCTTG
CCGATTTCTTAANCNTCTTNCCTACCCNTGNNNCAAGGGNAGGTATAN
NT,
PTCH cDNA

SEQ ID NO: 9

GCGCCCGCCGTGTGAGCAGCAGCAGCGGCTGGTCTGTCAACCGGAGCC
CGAGCCCGAGCAGCCTGCGGCCAGCAGCGTCCTCGCAAGCCGAGCGCC
CAGGCGCGCCAGGAGCCCGCAGCAGCGGCAGCAGCGCGCCGGGCCGCC
CGGGAAGCCTCCGTCCCCGCGGCGGCGGCGGCGGCGGCGGCAACATGG
CCTCGGCTGGTAACGCCGCCGAGCCCCAGGACCGCGGCGGCGGCGGCA
GCGGCTGTATCGGTGCCCCGGGACGGCCGGCTGGAGGCGGGAGGCGCA
GACGGACGGGGGGGCTGCGCCGTGCTGCCGCGCCGGACCGGGACTATC
TGCACCGGCCCAGCTACTGCGACGCCGCCTTCGCTCTGGAGCAGATTT
CCAAGGGGAAGGCTACTGGCCGGAAAGCGCCGCTGTGGCTGAGAGCGA
AGTTTCAGAGACTCTTATTTAAACTGGGTTGTTACATTCAAAAAAACT
GCGGCAAGTTCTTGGTTGTGGGCCTCCTCATATTTGGGGCCTTCGCGG
TGGGATTAAAAGCAGCGAACCTCGAGACCAACGTGGAGGAGCTGTGGG
TGGAAGTTGGAGGACGAGTAAGTCGTGAATTAAATTATACTCGCCAGA
AGATTGGAGAAGAGGCTATGTTTAATCCTCAACTCATGATACAGACCC
CTAAAGAAGAAGGTGCTAATGTCCTGACCACAGAAGCGCTCCTACAAC
ACCTGGACTCGGCACTCCAGGCCAGCCGTGTCCATGTATACATGTACA
ACAGGCAGTGGAAATTGGAACATTTGTGTTACAAATCAGGAGAGCTTA
TCACAGAAACAGGTTACATGGATCAGATAATAGAATATCTTTACCCTT
GTTTGATTATTACACCTTTGGACTGCTTCTGGGAAGGGGCGAAATTAC
AGTCTGGGACAGCATACCTCCTAGGTAAACCTCCTTTGCGGTGGACAA
ACTTCGACCCTTTGGAATTCCTGGAAGAGTTAAAGAAAATAAACTATC
AAGTGGACAGCTGGGAGGAAATGCTGAATAAGGCTGAGGTTGGTCATG
GTTACATGGACCGCCCCTGCCTCAATCCGGCCGATCCAGACTGCCCCG
CCACAGCCCCCAACAAAAATTCAACCAAACCTCTTGATATGGCCCTTG
TTTTGAATGGTGGATGTCATGGCTTATCCAGAAAGTATATGCACTGGC
AGGAGGAGTTGATTGTGGGTGGCACAGTCAAGAACAGCACTGGAAAAC
TCGTCAGCGCCCATGCCCTGCAGACCATGTTCCAGTTAATGACTCCCA
AGCAAATGTACGAGCACTTCAAGGGGTACGAGTATGTCTCACACATCA
ACTGGAACGAGGACAAAGCGGCAGCCATCCTGGAGGCCTGGCAGAGGA
CATATGTGGAGGTGGTTCATCAGAGTGTCGCACAGAACTCCACTCAAA
AGGTGCTTTCCTTCACCACCACGACCCTGGACGACATCCTGAAATCCT
TCTCTGACGTCAGTGTCATCCGCGTGGCCAGCGGCTACTTACTCATGC
TCGCCTATGCCTGTCTAACCATGCTGCGCTGGGACTGCTCCAAGTCCC
AGGGTGCCGTGGGGCTGGCTGGCGTCCTGCTGGTTGCACTGTCAGTGG
CTGCAGGACTGGGCCTGTGCTCATTGATCGGAATTTCCTTTAACGCTG
CAACAACTCAGGTTTTGCCATTTCTCGCTCTTGGTGTTGGTGTGGATG
ATGTTTTTCTTCTGGCCCACGCCTTCAGTGAAACAGGACAGAATAAAA
GAATCCCTTTTGAGGACAGGACCGGGGAGTGCCTGAAGCGCACAGGAG
CCAGCGTGGCCCTCACGTCCATCAGCAATGTCACAGCCTTCTTCATGG
CCGCGTTAATCCCAATTCCCGCTCTGCGGGCGTTCTCCCTCCAGGCAG
CGGTAGTAGTGGTGTTCAATTTTGCCATGGTTCTGCTCATTTTTCCTG
CAATTCTCAGCATGGATTTATATCGACGCGAGGACAGGAGACTGGATA
TTTTCTGCTGTTTTACAAGCCCCTGCGTCAGCAGAGTGATTCAGGTTG
AACCTCAGGCCTACACCGACACACACGACAATACCCGCTACAGCCCCC
CACCTCCCTACAGCAGCCACAGCTTTGCCCATGAAACGCAGATTACCA
TGCAGTCCACTGTCCAGCTCCGCACGGAGTACGACCCCCACACGCACG
TGTACTACACCACCGCTGAGCCGCGCTCCGAGATCTCTGTGCAGCCCG
TCACCGTGACACAGGACACCCTCAGCTGCCAGAGCCCAGAGAGCACCA
GCTCCACAAGGGACCTGCTCTCCCAGTTCTCCGACTCCAGCCTCCACT
GCCTCGAGCCCCCCTGTACGAAGTGGACACTCTCATCTTTTGCTGAGA
AGCACTATGCTCCTTTCCTCTTGAAACCAAAAGCCAAGGTAGTGGTGA
TCTTCCTTTTTCTGGGCTTGCTGGGGGTCAGCCTTTATGGCACCACCC
GAGTGAGAGACGGGCTGGACCTTACGGACATTGTACCTCGGGAAACCA
GAGAATATGACTTTATTGCTGCACAATTCAAATACTTTTCTTTCTACA
ACATGTATATAGTCACCCAGAAAGCAGACTACCCGAATATCCAGCACT
TACTTTACGACCTACACAGGAGTTTCAGTAACGTGAAGTATGTCATGT
TGGAAGAAAACAAACAGCTTCCCAAAATGTGGCTGCACTACTTCAGAG
ACTGGCTTCAGGGACTTCAGGATGCATTTGACAGTGACTGGGAAACCG
GGAAAATCATGCCAAACAATTACAAGAATGGATCAGACGATGGAGTCC
TTGCCTACAAACTCCTGGTGCAAACCGGCAGCCGCGATAAGCCCATCG
ACATCAGCCAGTTGACTAAACAGCGTCTGGTGGATGCAGATGGCATCA
TTAATCCCAGCGCTTTCTACATCTACCTGACGGCTTGGGTCAGCAACG
ACCCCGTCGCGTATGCTGCCTCCCAGGCCAACATCCGGCCACACCGAC
CAGAATGGGTCCACGACAAAGCCGACTACATGCCTGAAACAAGGCTGA
GAATCCCGGCAGCAGAGCCCATCGAGTATGCCCAGTTCCCTTTCTACC
TCAACGGCTTGCGGGACACCTCAGACTTTGTGGAGGCAATTGAAAAAG
TAAGGACCATCTGCAGCAACTATACGAGCCTGGGGCTGTCCAGTTACC
CCAACGGCTACCCCTTCCTCTTCTGGGAGCAGTACATCGGCCTCCGCC
ACTGGCTGCTGCTGTTCATCAGCGTGGTGTTGGCCTGCACATTCCTCG
TGTGCGCTGTCTTCCTTCTGAACCCCTGGACGGCCGGGATCATTGTGA
TGGTCCTGGCGCTGATGACGGTCGAGCTGTTCGGCATGATGGGCCTCA
TCGGAATCAAGCTCAGTGCCGTGCCCGTGGTCATCCTGATCGCTTCTG
TTGGCATAGGAGTGGAGTTCACCGTTCACGTTGCTTTGGCCTTTCTGA
CGGCCATCGGCGACAAGAACCGCAGGGCTGTGCTTGCCCTGGAGCACA
TGTTTGCACCCGTCCTGGATGGCGCCGTGTCCACTCTGCTGGGAGTGC
TGATGCTGGCGGGATCTGAGTTCGACTTCATTGTCAGGTATTTCTTTG
CTGTGCTGGCGATCCTCACCATCCTCGGCGTTCTCAATGGGCTGGTTT
TGCTTCCCGTGCTTTTGTCTTTCTTTGGACCATATCCTGAGGTGTCTC
CAGCCAACGGCTTGAACCGCCTGCCCACACCCTCCCCTGAGCCACCCC
CCAGCGTGGTCCGCTTCGCCATGCCGCCCGGCCACACGCACAGCGGGT
CTGATTCCTCCGACTCGGAGTATAGTTCCCAGACGACAGTGTCAGGCC
TCAGCGAGGAGCTTCGGCACTACGAGGCCCAGCAGGGCGCGGGAGGCC
CTGCCCACCAAGTGATCGTGGAAGCCACAGAAAACCCCGTCTTCGCCC
ACTCCACTGTGGTCCATCCCGAATCCAGGCATCACCCACCCTCGAACC
CGAGACAGCAGCCCCACCTGGACTCAGGGTCCCTGCCTCCCGGACGGC
AAGGCCAGCAGCCCCGCAGGGACCCCCCCAGAGAAGGCTTGTGGCCAC
CCCTCTACAGACCGCGCAGAGACGCTTTTGAAATTTCTACTGAAGGGC
ATTCTGGCCCTAGCAATAGGGCCCGCTGGGGCCCTCGCGGGGCCCGTT
CTCACAACCCTCGGAACCCAGCGTCCACTGCCATGGGCAGCTCCGTGC
CCGGCTACTGCCAGCCCATCACCACTGTGACGGCTTCTGCCTCCGTGA
CTGTCGCCGTGCACCCGCCGCCTGTCCCTGGGCCTGGGCGGAACCCCC
GAGGGGGACTCTGCCCAGGCTACCCTGAGACTGACCACGGCCTGTTTG
AGGACCCCCACGTGCCTTTCCACGTCCGGTGTGAGAGGAGGGATTCGA
AGGTGGAAGTCATTGAGCTGCAGGACGTGGAATGCGAGGAGAGGCCCC
GGGGAAGCAGCTCCAACTGAGGGTGATTAAAATCTGAAGCAAAGAGGC
CAAAGATTGGAAACCCCCCACCCCCACCTCTTTCCAGAACTGCTTGAA
GAGAACTGGTTGGAGTTATGGAAAAGATGCCCTGTGCCAGGACAGCAG
TTCATTGTTACTGTAACCGATTGTATTATTTTGTTAAATATTTCTATA
AATATTTAAGAGATGTACACATGTGTAATATAGGAAGGAAGGATGTAA
AGTGGTATGATCTGGGGCTTCTCCACTCCTGCCCCAGAGTGTGGAGGC
CACAGTGGGGCCTCTCCGTATTTGTGCATTGGGCTCCGTGCCACAACC
AAGCTTCATTAGTCTTAAATTTCAGCATATGTTGCTGCTGCTTAAATA
TTGTATAATTTACTTGTATAATTCTATGCAAATATTGCTTATGTAATA
GGATTATTTTGTAAAGGTTTCTGTTTAAAATATTTTAAATTTGCATAT
CACAACCCTGTGGTAGTATGAAATGTTACTGTTAACTTTCAAACACGC
TATGCGTGATAATTTTTTTGTTTAATGAGCAGATATGAAGAAAGCACG
TTAATCCTGGTGGCTTCTCTAGGTGTCGTTGTGTGCGGTCCTCTTGTT
TGGCTGTGCGTGTGAACACGTGTGTGAGTTCACCATGTACTGTACTGT
GATTTTTTTTTTGTCTTGTTTTGTTTCTCTACACTGTCTGTAACCTGT
AGTAGGCTCTGACCTAGTCAGGCTGGAAGCGTCAGGATATCTTTTCTT
CGTGCTGGTGAGGGCTGGCCCTAAACATCCACCTAATCCTTTCAAATC
AGCCCGGCAAAAGCTAGACTCTCCTCGTGTCTACGGCATCTCTTATGA
TCATTGGCTGCCATCCAGGACCCCAATTTGTGCTTCAGGGGGATAATC
TCCTTCTCTCGGATCATTGTGATGGATGCTGGAACCTCAGGGTATGGA
GCTCACATCAGTTCATCATGGTGGGTGTTAGAGAATTCGGTGACATGC
CTAGTGCTGAGCCTTGGCTGGGCCATGAGAGTCTGTATACTCTAAAAA
GCATGCAGCATGGTGCCCCTCTTCTGACCAACACACACACGACCCCTC
CCCCAACACCCCCAAATTCAAGAGTGGATGTGGCCCTGTCACAGGTAG
AAAAACCTATTTAGTTAATTCTTTCTTGGCCCACAGTCTCCCAGAAAT
GATGTTTTGAGTCCCTATAGTTTAAACTCCCTCTCTTAAATGGAGCAG
CTGGTTGAGGCTTTCTAGATCTGTTTGCATCTTCTTTAAAACTAAGTG
GTGAGCATGCATTGTGGTGTAGAGGCAGGCATTATGTAGGATAAGAGC
TCCGGGGGGATTCTTCATGCACCAGTGTTTAGGGTACGTGCTTCCTAA
GTAAATCCAAACATTGTCTCCATCCTCCCCGTCATTAGTGCTCTTTCA
ATGTGATGTGGGAAAGCAGGAGGATGGACACACCCCACTGAAAGATGT
AGGCAGGGGCAGGTCTCTCAACCAGGCATATTTTTAAAAGTTGCTTCT
GTACTGGTTCTCTTCTTTTGCTCTGAGGTGTGGGCTCCCTCATCTCGT
AACCAGAGACCAGCACATGTCAGGGAAGCACCCAGTGTCGGCTCCCCA
TCCAAATCCACACCAGCACCTTGTTACAGACAAGAAGTCAGAGGAAAG
GGCGGGGTCCCTGCAGGGCTGAAGCCTAAGCTACTGTGAGGCGCTCAC
GAGTGGCAGCTCCTGTTACTCCCTTTTAAATTACCTGGGAAATCTTAA
CAGAAAGGTAATGGGCCCCCAGAAATACCCACAGCATAGTGACCTCAG
ACCCTGATACTCACCACAAAACTTTTAAGATGCTGATTGGGAGCCGCT
TGTGGCTGCTGGGTGTGTGTGTGTGTGTGTGCGTGCGTGCGTGTGTGT
GTGTCTCTGCTGGGGACCCTGGCCACCCCCCTGCTGCTGTCTTGGTGC
CTGTCACCCACATGGTCTGCCATCCTAACACCCAGCTCTGCTCAGAAA
ACGTCCTGCGTGGAGGAGGGATGATGCAGAATTCTGAAGTCGACTTCC
CTCTGGCTCCTGGCGTGCCCTCGCTCCCTTCCTGAGCCCAGCTCGTGT
TGCGCCGGAGGCTGCGCGGCCCCTGATTTCTGCATGGTGTAGAACTTT
CTCCAATAGTCACATTGGCAAAGGGAGAACTGGGGTGGGCGGGGGGTG
GGGCTGGCAGGGAATTAGAATTTCTCTCTCTCTTTTAATAGTTTTATT
TTGTCTGTCCTGTTTGTTCATTTGGATGTTTTAATTTTTAAAAAAAAA
AAAAAAAAA,
PTCH protein

SEQ ID NO: 17

MASAGNAAEPQDRGGGGSGCIGAPGRPAGGGRRRRTGGLRRAAAPDRD
YLHRPSYCDAAFALEQISKGKATGRKAPLWLRAKFQRLLFKLGCYIQK
NCGKFLVVGLLIFGAFAVGLKAANLETNVELLWVEVGGRVSRELNYTR
QKIGEEAMFNPQLMIQTPKEEGANVLTTEALLQHLDSALQASRVHVYM
YNRQWKLEHLCYKSGELITETGYMDQIIEYLYPCLIITPLDCFWEGAK
LQSGTAYLLGKPPLRWTNFDPLEFLEELKKINYQVDSWEEMLNKAEVG
HGYMDRPCLNPADPDCPATAPNKNSTKPLDMALVLNGGCHGLSRKYMH
WQEELIVGGTVKSTGKLVSAHALQTMFQLMTPKQMYEHFKGYEYVSHI
NWNEDKAAAILEAWQRTYVEVVHQSVAQNSTQKVLSFTTTTLDDILKS
FSDVSVIRVASGYLLMLAYACLTMLRWDCSKSQGAVGLAGVLLVALSV
AAGLGLCSLIGISFNAATTQVLPFLALGVGVDDVFLLAHAFSETGQNK
RIPFEDRTGECLKRTGASVALTSISNVTAFFMAALIPIPALRAFSLQA
AVVVVFNFAMVLLIFPAILSMDLYRREDRRLDIFCCFTSPCVSRVIQV
EPQAYTDTHDNTRYSPPPPYSSHSFAHETQITMQSTVQLRTEYDPHTH
VYYTTAEPRSEISVQPVTVTQDTLSCQSPESTSSTRDLLSQFSDSSLH
CLEPPCTKWTLSSFAEKHYAPFLLKPKAKVVVIFLFLGLLGVSLYGTT
RVRDGLDLTDIVPRETREYDFIAAQFKYFSFYNMYIVTQKADYPNIQH
LLYDLHRSFSNVKYVMLEENKQLPKMWLHYFRDWLQGLQDAFDSDWET
GKIMPNNYKNGSDDGVLAYKLLVQTGSRDKPIDISQLTKQRLVDADGI
INPSAFYIYLTAWVSNDPVAYAASQANIRPHRPEWVHDKADYMPETRL
RIPAAEPIEYAQFPFYLNGLRDTSDFVEAIEKVRTICSNYTSLGLSSY
PNGYPFLFWEQYIGLRHWLLLFISVVLACTFLVCAVFLLNPWTAGIIV
MVLALMTVELFGMMGLIGIKLSAVPVVILIASVGIGVEFTVHVALAFL
TAIGDKNRRAVLALEHMFAPVLDGAVSTLLGVLMLAGSEFDFIVRYFF
AVLAILTILGVLNGLVLLPVLLSFFGPYPEVSPANGLNRLPTPSPEPP
PSVVRFAMPPGHTHSGSDSSDSEYSSQTTVSGLSEELRHYEAQQGAGG
PAHQVIVEATENPVFAHSTVVHPESRHHPPSNPRQQPHLDSGSLPPGR
QGQQPRRDPPREGLWPPLYRPRRDAFEISTEGHSGPSNRARWGPRGAR
SHNPRNPASTAMGSSVPGYCQPITTVTASASVTVAVHPPPVPGPGRNP
RGGLCPGYPETDHGLFEDPHVPFHVRCERRDSKVEVIELQDVECEERP
RGSSSN
PSMD2-flanking

SEQ ID NO: 2

CTTCTTCNTGACTCCTGGATTTCCTCTGTTCNCAACGGGACACAGCCT
TACCAAATTCAAACGGCCGAGAGGACGTTATGTATCATCTAGAACTAA
TCCTGACTTCAACAGTGTCCTTCACACCCCTTCTAAGTCAAATCACGG
AAAGACTCAAAAGACAGAGATTGAAGAAGGCAAAGCCTGTGTCTTGAT
CTGCCTTTAGTTCTAGAGTTTAGCATCNGAGCATANGACCACATTGTA
TTGATGGACTCCGACCAGGNTCCGCAGGNGGATTTAAGGTGGGGGCCG
TACGCGGCAGGTGGTACCCGACCACTCTCCTTCACCNNGGGGTAAAAC
GTTACGAGGTTAATATTCCGCGGCGGCGGAAGTAGATACAGGTTGCAG
ATCTCACACGGGCGGCGATCAAGCATTCCGGACGTGAAGAGTCTCGTT
CGTCTGTCCCACCACGCAGCCGACTGCGGTGTCACTGTGGGTACCGGT
CGCTCGGCNAGTAAGGAGACCCCGCGGGCGGNCCCTCGGNTCGCGGCT
CTTCATCTCCTACCGCAGCCAGCGGACTCGGATCNCAGACTGCACGGC
CNCATGGCCTTCCGGAAACTCCCGGTCCGAGCCGGGGCGGCGCCTGGG
GCGNATNAACNGTTAGAACTTGCAGTTTTGGGGGCGGNCTCCGAGGGN
GGGGGTCCAGGGCCCGGGCCTCNCGAAA,
PSMD2-cDNA

SEQ ID NO: 10

TGCGCGCGCAGCGGGCCGGCAGTGGCGGCGGAGATGGAGGAGGGAGGC
CGGGACAAGGCGCCGGTGCAGCCCCAGCAGTCTCCAGCGGCGGCCCCC
GGCGGCACGGACGAGAAGCCGAGCGGCAAGGAGCGGCGGGATGCCGGG
GACAAGGACAAAGAACAGGAGCTGTCTGAAGAGGATAAACAGCTTCAA
GATGAACTGGAGATGCTCGTGGAACGACTAGGGGAGAAGGATACATCC
CTGTATCGACCAGCGCTGGAGGAATTGCGAAGGCAGATTCGTTCTTCT
ACAACTTCCATGACTTCAGTGCCCAAGCCTCTCAAATTTCTGCGTCCA
CACTATGGCAAACTGAAGGAAATCTATGAGAACATGGCCCCTGGGGAG
AATAAGCGTTTTGCTGCTGACATCATCTCCGTTTTGGCCATGACCATG
AGTGGGGAGCGTGAGTGCCTCAAGTATCGGCTAGTGGGCTCCCAGGAG
GAATTGGCATCATGGGGTCATGAGTATGTCAGGCATCTGGCAGGAGAA
GTGGCTAAGGAGTGGCAGGAGCTGGATGACGCAGAGAAGGTCCAGCGG
GAGCCTCTGCTCACTCTGGTGAAGGAAATCGTCCCCTATAACATGGCC
CACAATGCAGAGCATGAGGCTTGCGACCTGCTTATGGAAATTGAGCAG
GTGGACATGCTGGAGAAGGACATTGATGAAAATGCATATGCAAAGGTC
TGCCTTTATCTCACCAGTTGTGTGAATTACGTGCCTGAGCCTGAGAAC
TCAGCCCTACTGCGTTGTGCCCTGGGTGTGTTCCGAAAGTTTAGCCGC
TTCCCTGAAGCTCTGAGATTGGCATTGATGCTCAATGACATGGAGTTG
GTAGAAGACATCTTCACCTCCTGCAAGGATGTGGTAGTACAGAAACAG
ATGGCATTCATGCTAGGCCGGCATGGGGTGTTCCTGGAGCTGAGTGAA
GATGTCGAGGAGTATGAGGACCTGACAGAGATCATGTCCAATGTACAG
CTCAACAGCAACTTCTTGGCCTTAGCTCGGGAGCTGGACATCATGGAG
CCCAAGGTGCCTGATGACATCTACAAAACCCACCTAGAGAACAACAGG
TTTGGGGGCAGTGGCTCTCAGGTGGACTCTGCCCGCATGAACCTGGCC
TCCTCTTTTGTGAATGGCTTTGTGAATGCAGCTTTTGGCCAAGACAAG
CTGCTAACAGATGATGGCAACAAATGGCTTTACAAGAACAAGGACCAC
GGAATGTTGAGTGCAGCTGCATCTCTTGGGATGATTCTGCTGTGGGAT
GTGGATGGTGGCCTCACCCAGATTGACAAGTACCTGTACTCCTCTGAG
GACTACATTAAGTCAGGAGCTCTTCTTGCCTGTGGCATAGTGAACTCT
GGGGTCCGGAATGAGTGTGACCCTGCTCTGGCACTGCTCTCAGACTAT
GTTCTCCACAACAGCAACACCATGAGACTTGGTTCCATCTTTGGGCTA
GGCTTGGCTTATGCTGGCTCAAATCGTGAAGATGTCCTAACACTGCTG
CTGCCTGTGATGGGAGATTCAAAGTCCAGCATGGAGGTGGCAGGTGTC
ACAGCTTTAGCCTGTGGAATGATAGCAGTAGGGTCCTGCAATGGAGAT
GTAACTTCCACTATCCTTCAGACCATCATGGAGAAGTCAGAGACTGAG
CTCAAGGATACTTATGCTCGTTGGCTTCCTCTTGGACTGGGTCTCAAC
CACCTGGGGAAGGGTGAGGCCATCGAGGCAATCCTGGCTGCACTGGAG
GTTGTGTCAGAGCCATTCCGCAGTTTTGCCAACACACTGGTGGATGTG
TGTGCATATGCAGGCTCTGGGAATGTGCTGAAGGTGCAGCAGCTGCTC
CACATTTGTAGCGAACACTTTGACTCCAAAGAGAAGGAGGAAGACAAA
GACAAGAAGGAAAAGAAAGACAAGGACAAGAAGGAAGCCCCTGCTGAC
ATGGGAGCACATCAGGGAGTGGCTGTTCTGGGGATTGCCCTTATTGCT
ATGGGGGAGGAGATTGGTGCAGAGATGGCATTACGAACCTTTGGCCAC
TTGCTGAGATATGGGGAGCCTACACTCCGGAGGGCTGTACCTTTAGCA
CTGGCCCTCATCTCTGTTTCAAATCCACGACTCAACATCCTGGATACC
CTAAGCAAATTCTCTCATGATGCTGATCCAGAAGTTTCCTATAACTCC
ATTTTTGCCATGGGCATGGTGGGCAGTGGTACCAATAATGCCCGTCTG
GCTGCAATGCTGCGCCAGTTAGCTCAATATCATGCCAAGGACCCAAAC
AACCTCTTCATGGTGCGCTTGGCACAGGGCCTGACACATTTAGGGAAG
GGCACCCTTACCCTCTGCCCCTACCACAGCGACCGGCAGCTTATGAGC
CAGGTGGCCGTGGCTGGACTGCTCACTGTGCTTGTCTCTTTCCTGGAT
GTTCGAAACATTATTCTAGGCAAATCACACTATGTATTGTATGGGCTG
GTGGCTGCCATGCAGCCCCGAATGCTGGTTACGTTTGATGAGGAGCTG
CGGCCATTGCCAGTGTCTGTCCGTGTGGGCCAGGCAGTGGATGTGGTG
GGCCAGGCTGGCAAGCCGAAGACTATCACAGGGTTCCAGACGCATACA
ACCCCAGTGTTGTTGGCCCACGGGGAACGGGCAGAATTGGCCACTGAG
GAGTTTCTTCCTGTTACCCCCATTCTGGAAGGTTTTGTTATCCTTCGG
AAGAACCCCAATTATGATCTCTAAGTGACCACCAGGGGCTCTGAACTG
CAGCTGATGTTATCAGCAGGCCATGCATCCTGCTGCCAAGGGTGGACA
CGGCTGCAGACTTCTGGGGGAATTGTCGCCTCCTGCTCTTTTGTTACT
GAGTGAGATAAGGTTGTTCAATAAAGACTTTTATCCCCAAGGAAAAAA
AAAAAAAAAAAAAAAAAAAAAAAAAAAAAAAAAAAAAAAAAAAAAAAA
AAAAAAAAAAAAAA,
PSDM2 protein

SEQ ID NO; 18

MEEGGRDKAPVQPQQSPAAAPGGTDEKPSGKERRDAGDKDKEQELSEE
DKQLQDELEMLVERLGEKDTSLYRPALEELRRQIRSSTTSMTSVPKPL
KFLRPHYGKLKEIYENMAPGENKRFAADIISVLAMTMSGERECLKYRL
VGSQEELASWGHEYVRHLAGEVAKEWQELDDAEKVQREPLLTLVKEIV
PYNMAHNAEHEACDLLMEIEQVDMLEKDIDENAYAKVCLYLTSCVNYV
PEPENSALLRCALGVFRKFSRFPEALRLALMLNDMELVEDIFTSCKDV
VVQKQMAFMLGRHGVFLELSEDVEEYEDLTEIMSNVQLNSNFLALARE
LDIMEPKVPDDIYKTHLENNRFGGSGSQVDSARMNLASSFVNGFVNAA
FGQDKLLTDDGNKWLYKNKDHGMLSAAASLGMILLWDVDGGLTQIDKY
LYSSEDYIKSGALLACGIVNSGVRNECDPALALLSDYVLHNSNTMRLG
SIFGLGLAYAGSNREDVLTLLLPVMGDSKSSMEVAGVTALACGMIAVG
SCNGDVTSTILQTIMEKSETELKDTYARWLPLGLGLNHLGKGEAIEAI
LAALEVVSEPFRSFANTLVDVCAYAGSGNVLKVQQLLHICSEHFDSKE
KEEDKDKKEKKDKDKKEAPADMGAHQGVAVLGIALIAMGEEIGAEMAL
RTFGHLLRYGEPTLRRAVPLALALISVSNPRLNILDTLSKFSHDADPE
VSYNSIFAMGMVGSGTNNARLAAMLRQLAQYHAKDPNNLFMVRLAQGL
THLGKGTLTLCPYHSDRQLMSQVAVAGLLTVLVSFLDVRNIILGKSHY
VLYGLVAAMQPRMLVTFDEELRPLPVSVRVGQAVDVVGQAGKPKTITG
FQTHTTPVLLAHGERAELATEEFLPVTPILEGFVILRKNPNYDL,
NMT1 flanking

SEQ ID NO: 3

GTCTCCAGTTTAGGGAACCATGGGGGAAGGAAGAAAAGTCGCGCANTA
TCATGCCATCCTGCGTTTGCGCNAATGGATGGGTGGGAATCCCATGCT
GCCACNNANGNCCGGGGGAAAAGAGGTGTTTTCTCTTAAAATTTTNTA
NCCGGTCNAGCCNCTGGGGAAAATGTAAGGGGAGGCNAAGCCTTCTGA
AAAGTGGAGATGATNACTCAGCGAAACAAAAGTACNCATTNAANCACT
TTTAATTCACTCTATGANATAGGTACCATTCCCGNTTTCCAGATGAGC
AAACTGAGAGTCAGAAAGGTACGCAAGTTGACNGAAATGGAAAGGNCN
NATGTTAGATNCAAAAATAAANGAGATCTGGGCAGCGGTGGNTCAGCG
NCTTANCGCCGCCTTNAGCCCAGGGCATGATCCTGGGGTCCCGGGATC
GAGTCCCACGTCGGGCTCCCTGCATGGAGCCTGCTTCTCCCTCTGCCT
GTGTCTCTCTCTGNGNCTATCANGAAATAAATAAGNTNNTAANATATC
ANATNTTAAAAAAATNNTCTCCCTCAGNATCTGCCCCCCNNAGTTTCT
TGAGTCCTAGNGGNCTTTTGGNACTGGAACCTGCCTGTATCTTCAACC
CACCTTTCTCAAATCNNNAGNTGNAAANNAGGNAANGGAACNCCTNCC
TNAACCGGGTGCCNTTNAGGGCTGATGACCCACNGTATTCCAGGCNNT
TTTACCCANGGGNTTGNNTCCAAANATCCNTGCTCCAACAATTNNANT
NAAAGGNTTGAA.
(NMT1) cDNA

SEQ ID NO: 11

CTGCTCTCGCAACTCAAGATGGCGGACGAGAGTGAGACAGCAGTGAAG
CCGCCGGCACCTCCGCTGCCGCAGATGATGGAAGGGAACGGGAACGGC
CATGAGCACTGCAGCGATTGCGAGAATGAGGAGGACAACAGCTACAAC
CGGGGTGGTTTGAGTCCAGCCAATGACACTGGAGCCAAAAAGAAGAAA
AAGAAACAAAAAAAGAAGAAAGAAAAAGGCAGTGAGACAGATTCAGCC
CAGGATCAGCCTGTGAAGATGAACTCTTTGCCAGCAGAGAGGATCCAG
GAAATACAGAAGGCCATTGAGCTGTTCTCAGTGGGTCAGGGACCTGCC
AAAACCATGGAGGAGGCTAGCAAGCGAAGCTACCAGTTCTGGGATACG
CAGCCCGTCCCCAAGCTGGGCGAAGTGGTGAACACCCATGGCCCCGTG
GAGCCTGACAAGGACAATATCCGCCAGGAGCCCTACACCCTGCCCCAG
GGCTTCACCTGGGATGCTTTGGACTTGGGCGATCGTGGTGTGCTAAAA
GAACTGTACACCCTCCTGAATGAGAACTATGTGGAAGATGATGACAAC
ATGTTCCGATTTGATTATTCCCCGGAGTTTCTTTTGTGGGCTCTCCGG
CCACCCGGCTGGCTCCCCCAGTGGCACTGTGGGGTTCGAGTGGTCTCA
AGTCGGAAATTGGTTGGGTTCATTAGCGCCATCCCAGCAAACATCCAT
ATCTATGACACAGAGAAGAAGATGGTAGAGATCAACTTCCTGTGTGTC
CACAAGAAGCTGCGTTCCAAGAGGGTTGCTCCAGTTCTGATCCGAGAG
ATCACCAGGCGGGTTCACCTGGAGGGCATCTTCCAAGCAGTTTACACT
GCCGGGGTGGTACTACCAAAGCCCGTTGGCACCTGCAGGTATTGGCAT
CGGTCCCTAAACCCACGGAAGCTGATTGAAGTGAAGTTCTCCCACCTG
AGCAGAAATATGACCATGCAGCGCACCATGAAGCTCTACCGACTGCCA
GAGACTCCCAAGACAGCTGGGCTGCGACCAATGGAAACAAAGGACATT
CCAGTAGTGCACCAGCTCCTCACCAGGTACTTGAAGCAATTTCACCTT
ACGCCCGTCATGAGCCAGGAGGAGGTGGAGCACTGGTTCTACCCCCAG
GAGAATATCATCGACACTTTCGTGGTGGAGAACGCAAACGGAGAGGTG
ACAGATTTCCTGAGCTTTTATACGCTGCCCTCCACCATCATGAACCAT
CCAACCCACAAGAGTCTCAAAGCTGCTTATTCTTTCTACAACGTTCAC
ACCCAGACCCCTCTTCTAGACCTCATGAGCGACGCCCTTGTCCTCGCC
AAAATGAAAGGGTTTGATGTGTTCAATGCACTGGATCTCATGGAGAAC
AAAACCTTCCTGGAGAAGCTCAAGTTTGGCATAGGGGACGGCAACCTG
CAGTATTACCTTTACAATTGGAAATGCCCCAGCATGGGGGCAGAGAAG
GTTGGACTGGTGCTACAATAACCAGTCACCAGTGCGATTCTGGATAAA
GCCACTGAAAATTCGAACCAGGAAATGGAACCCCACCACTGTTGGTCC
AATTTTCACACACGTGAGAATCCCTGGCAAAGGGAGCAGAACTGAACC
GGCTTTACCAAACCGCCAGCGAACTTGACAATTGTATTGCGATGGCGT
GGGCTGCGTGACGTCACCTCCGGTCGTGTCTCTGGTCTCCGTGTTTTC
CAGTTAATTACATCCTCATGCAGCCGTGATCAAGGGAATGTAACTGCT
GAAAACTAGCTCGTGATTGGCATATAATGGAGTTAACGGGTGAATAAT
AAAAGTATATATATATATTATATATATATAAATATTTTAAATATCTTT
CATGTTCCAAATGTACAAGGATGTTTGGTCTTTAATGAAAAGCTGAAT
CTAGATCATTCCTCAGAATGAGGACCCGAGGACAGTGGCAGACAGACG
CGTTGGCACAGTTCATGGTTTCCTCCAGAGGAGACATTGGCTTATCAT
GGGGAAAAAGAGGATCTGGAGAACCTCATCCAGCTCCCCTTCTGAATC
AGCTGGGATGACTGGCTTTGAGAAGGAAGGGAAGATGGAACAGGCTCA
GATCTCATGGGATAGCACGTGGAGCTCTTGGCTGGGGCTGACCCTGGG
CAGGGACTTTCCTGCAGGGCCAGACCTGCCTGCATTCTGAGACAAAGC
AATGGACGGTCCGCAGAAGCAGACCTCATTGATTGAGTCCTTTCTTCC
ATCCCCTTGGCCTGCTCCCTGTAGGAAGTCATCCTGCCAACTGATTTA
AAAGGGCTCTTTAGCCAGTTGTTGCCAACCTTATAGGGATGAGTCCCC
TGTGAGATTTTGCTTTTCCACTGCCTGGGATGATGCAGTTTGAAGAGG
CCCTTGGACCTCCTTGTAACATCAGGGACCTTTGGAGACCATTATCAG
TGTAAGCCCTGCTTAGCTCATCTTAGAGCAAAGAGCCAGCACCCTGAT
GTCCCTGGGGTGGCTAGGCAGGAGTGGCGTGGGGCCAATACCCAGACC
CCTTCAGCCACCAGCCCCTGGCCTGTGCCTTCCAACCCATTAGCCATT
TCTTGTTGTGCCCCTTTCCAAGATACAGCCTGCAAGTGGTAGCAAGAA
GTGATTAGAGGCAGATCTGGACTTGGCAACAGAAGTGGTTTCCCATCT
CCATTGTCTGAGTCTGATTTTCGCTGATGCTGTTTTGTGGATTTTTGT
GGTAGTGATGGTTGTCAGTGCTGCCAGTTTCCCAAAACGTAATCAAGC
CTCTGGTCACATGGCTGTCGATGTAGGCATTCTGGAGTGGTGTTCAGC
CAAGTGACCGGGCAAAATTGGGCTGTGAAATTGTACTTCCAGGCTTGG
ATGTAATTTTTGCTCTAGAGAGAAGCAAGTGGTGGGAAGGAGGTAGCA
TGACGTGTGGTGTGCGGGTTTCCTTGCTGCCGTCACCTCTCCGCTCAT
ACAGGAATGAAGCCTTAGCCAGGAGGCCAGGCTCAGCCCTGTGCCACT
CACCGAAGCCACTTTCTACAGGCCAGCAGGGGCTTGTTGCAGGCTGTG
GGTTTTGGTGTGGTTTGTCAGAGGCTAATTCTGCAGAGTTTCCAAAAC
CAGAAGACATCGTATGCTTGGGATGGGGGCCGTGCCACCCGTGGGAAT
GCTGCCCGCTCTGCAGACTGCTGCTAGAGCCAGCAACTCCACTAAGGT
GGATTTTCATCAGGGGCCTGCAGGGCCCTCCCTTTTCCCATTGTTCCT
GCGCTGCAAATTGCAGGCCCCAGCAATCGTGACTGACGTTTGCTCCTT
GACTCCAAGAAACTGAGACCAAAGAAGCTGCTGTTCTTAGCAAGATGC
GCACTGCATTCCACAGGTGGGAGGAGTCGGAGAGGCAGGGGCTTGCTT
TGCAGCCCCACAGACAACAGTTGCACAGTGCCTCAAGCCCCAGAGTGG
CTCACCCTGTCCAGACCTTTGAGGATATCAAAGGACAAAGTGCCCAAG
TCTTTCCTACCTTGGGGGAACCTGGAACTTGGAAAGGCTCCCTGTCCT
AGTCTTGATCTGTTCTGGGCCAGGTCCCAGCTTGAGCTGCCTCTGAGA
TTTGGGCTGTGCGGATCTCTGGAGTGAGCTCTGTTTCGGTTGACCCAG
GTCATGGAATGGAAACGGTGAGGCCCCAGTGGCTGTTCTGGAAGAAAC
AGATCTCCTGGCAAAGGCCCCAGCATCTCCCTCACTGAAACCAGGTGG
CCGGCTCCTCGGACTCTGCTTTATGTTGCGGTGAGAACTCTGCCCAGG
TGTGCAGGGTTTGGCTTGTGGGCTGCTTGCTGCTCATCTGATTTTTGT
CCCAGTAGTCCCTGCGTTCTTCATTCAACCCCTTCTGGGACTTCAGCT
CAGAGAGCACCATCCCGGGGGTCAGGGCCTCCCCACAGGAGCCCTGCA
GTGTGGTAGCGCCATGGCTGTCTCAAACCAAGCAAAGGAAGGACCCTG
AGGCCTTCACGCTAACCATCCTCGAGCAACTGCTGTTGGAAGGCCTCC
CTGGGCCTGGCCCCCACCCTCTGCCACCCAGTCCTCCCAGCTGCCATG
TTTCAAAGACGACCTTTACCTCCTGCCTTTGGATTGACTCTGCATTTG
ACCACGGACTCCAGTCTGTGTGTAGGGAGAGAGCTGAGTAGGAGGCCT
CCACTCCGGATCGAGGCCTGTATAGGGCTCGTTTCCCCACACATGCCT
ATTTCTGAAGAGGCTTCTGTCTTATTTGAAGGCCAGCCCACACCCAGC
TACTTTAACACCAGGTTTATGGAAAATGTCAGGCCTTCCCCACAACTC
CTGTCTAACTGCTGTCGCCCCCCTACTTGCTGGCTCTCAGAAGCCTAG
GGGAGTCCCTGTGGTCCTGAATTCTTTCCCCAAAGACGACCAGCATTT
AACCAACCTAAGGGCCCAAAGGCCTTGGACAACTGCATGGAGCTGCAC
TCTAGGAGAAGGAGGGGAACCAGATGTTAGATCAGGGGAGGGAGCAGG
AGTGTCCCTCCCGTCAGTGCCTACCCACCTGTGAGGCAGCCTTCTGAT
GGCCTGGCCCACCTTCCCCAGAACCAGGGGAGGCCTGAGGCTTCAGTT
TTACTCTGCTGCAAAATGAAGGCGGGCCTGCAAGCCGACTACACCTAC
GGAGGCTGTTGAGGACAATTTCATTCCATTAAATTAAAAAATACTGAC
TGGCTGGCAGGCAGGTGCCATGTCTGGGAACAGGGACGGGGGAGCTTC
ACCTTTTTGTCTTGGCTTTTCTTTGGGCTGTGGGGGGGCATCCATTTC
CAGGGTCGGGGAGGAAATACCAAATGCATTGTTGTTCTGCTCAATACA
TCTCACTTGTTTCTAATAAAGAAAGCAGCTGAACAAAAAAAAAAAAAA
AAAAAAA
NO: 19 protein (NMT1)
MADESETAVKPPAPPLPQMMEGNGNGHEHCSDCENEEDNSYNRGGLSP
ANDTGAKKKKKKQKKKKEKGSETDSAQDQPVKMNSLPAERIQEIQKAI
ELFSVGQGPAKTMEEASKRSYQFWDTQPVPKLGEVVNTHGPVEPDKDN
IRQEPYTLPQGFTWDALDLGDRGVLKELYTLLNENYVEDDDNMFRFDY
SPEFLLWALRPPGWLPQWHCGVRVVSSRKLVGFISAIPANIHIYDTEK
KMVEINFLCVHKKLRSKRVAPVLIREITRRVHLEGIFQAVYTAGVVLP
KPVGTCRYWHRSLNPRKLIEVKFSHLSRNMTMQRTMKLYRLPETPKTA
GLRPMETKDIPVVHQLLTRYLKQFHLTPVMSQEEVEHWFYPQENIIDT
FVVENANGEVTDFLSFYTLPSTIMNHPTHKSLKAAYSFYNVHTQTPLL
DLMSDALVLAKMKGFDVFNALDLMENKTFLEKLKFGIGDGNLQYYLYN
WKCPSMGAEKVGLVLQ
NO: 4, Macro flanking
CTGGTGCTGCCCTCTCTTCCACCCACTCACTCACCTTTCTCTGGTCAT
CTTGAATTCCTACAGTTTATCAATGCTGTTCCTTCAATTGAACGACTT
CTCTCACTCCCAAATCCCTTCTGGTGAATGACTATCACTCATCCTAAG
GGCACCTTTTCAATGAATCCTACTGCCAAGTAGAACTGACCCCTCACA
CTCCCAATCCATCTTTTCAATGTATATTCTGCACAGAGATTCCTCAAT
AGCACAAATAACTCTACAAGTTGGTTGTTTTTTCTTTCTTTTTTTAGA
GATTTTATTTAAGAAAGAGAGAGAGAGAACACAAGAGGGAGGGAGAGG
CAACAAGAGAGGAAAAAACAGATTCCCTGCTGAACAGGGAGCTCAAAG
CGGGGCTCAGTCTTAGTACCCTGAGACCATGACCTGAACAGAAGGCAG
ATGGTTAACTGAATGAGCCACCGAGGTGCCCCAGTGGTTGCTTTTATT
GGTCTCTTCCCGACTGTGAGTTCCCCAAGAGCAGGAACCACACATTAC
ATTGCTTAAACCTCAGTTCAAGCAGGAATAAAGAAGNGAAAGGATGAT
GGNAATTATCCAAACNCTGAGGAGCAAACCCCACGCANCATGCC
NO: 12 MACRO cDNA
GGGGGCCAAAGGGAAGTGCTGCGAGGTTTACAACCAGCTGCAGTGGTT
CGATGGGAAGGATCTTTCTCCAAGTGGTTCCTCTTGAGGGGAGCATTT
CTGCTGGCTCCAGGACTTTGGCCATCTATAAAGCTTGGCAATGAGAAA
TAAGAAAATTCTCAAGGAGGACGAGCTCTTGAGTGAGACCCAACAAGC
TGCTTTTCACCAAATTGCAATGGAGCCTTTCGAAATCAATGTTCCAAA
GCCCAAGAGGAGAAATGGGGTGAACTTCTCCCTAGCTGTGGTGGTCAT
CTACCTGATCCTGCTCACCGCTGGCGCTGGGCTGCTGGTGGTCCAAGT
TCTGAATCTGCAGGCGCGGCTCCGGGTCCTGGAGATGTATTTCCTCAA
TGACACTCTGGCGGCTGAGGACAGCCCGTCCTTCTCCTTGCTGCAGTC
AGCACACCCTGGAGAACACCTGGCTCAGGGTGCATCGAGGCTGCAAGT
CCTGCAGGCCCAACTCACCTGGGTCCGCGTCAGCCATGAGCACTTGCT
GCAGCGGGTAGACAACTTCACTCAGAACCCAGGGATGTTCAGAATCAA
AGGTGAACAAGGCGCCCCAGGTCTTCAAGGCCACAAGGGGGCCATGGG
CATGCCTGGTGCCCCTGGCCCGCCGGGACCACCTGCTGAGAAGGGAGC
CAAGGGGGCTATGGGACGAGATGGAGCAACAGGCCCCTCGGGACCCCA
AGGCCCACCGGGAGTCAAGGGAGAGGCGGGCCTCCAAGGACCCCAGGG
TGCTCCAGGGAAGCAAGGAGCCACTGGCACCCCAGGACCCCAAGGAGA
GAAGGGCAGCAAAGGCGATGGGGGTCTCATTGGCCCAAAAGGGGAAAC
TGGAACTAAGGGAGAGAAAGGAGACCTGGGTCTCCCAGGAAGCAAAGG
GGACAGGGGCATGAAAGGAGATGCAGGGGTCATGGGGCCTCCTGGAGC
CCAGGGGAGTAAAGGTGACTTCGGGAGGCCAGGCCCACCAGGTTTGGC
TGGTTTTCCTGGAGCTAAAGGAGATCAAGGACAACCTGGACTGCAGGG
TGTTCCGGGCCCTCCTGGTGCAGTGGGACACCCAGGTGCCAAGGGTGA
GCCTGGCAGTGCTGGCTCCCCTGGGCGAGCAGGACTTCCAGGGAGCCC
CGGGAGTCCAGGAGCCACAGGCCTGAAAGGAAGCAAAGGGGACACAGG
ACTTCAAGGACAGCAAGGAAGAAAAGGAGAATCAGGAGTTCCAGGCCC
TGCAGGTGTGAAGGGAGAACAGGGGAGCCCAGGGCTGGCAGGTCCCAA
GGGAGCCCCTGGACAAGCTGGCCAGAAGGGAGACCAGGGAGTGAAAGG
ATCTTCTGGGGAGCAAGGAGTAAAGGGAGAAAAAGGTGAAAGAGGTGA
AAACTCAGTGTCCGTCAGGATTGTCGGCAGTAGTAACCGAGGCCGGGC
TGAAGTTTACTACAGTGGTACCTGGGGGACAATTTGCGATGACGAGTG
GCAAAATTCTGATGCCATTGTCTTCTGCCGCATGCTGGGTTACTCCAA
AGGAAGGGCCCTGTACAAAGTGGGAGCTGGCACTGGGCAGATCTGGCT
GGATAATGTTCAGTGTCGGGGCACGGAGAGTACCCTGTGGAGCTGCAC
CAAGAATAGCTGGGGCCATCATGACTGCAGCCACGAGGAGGACGCAGG
CGTGGAGTGCAGCGTCTGACCCGGAAACCCTTTCACTTCTCTGCTCCC
GAGGTGTCCTCGGGCTCATATGTGGGAAGGCAGAGGATCTCTGAGGAG
TTCCCTGGGGACAACTGAGCAGCCTCTGGAGAGGGGCCATTAATAAAG
CTCAACATCAAAAAAAAAAAAGAAAAAAAAAAAAAAAAA
NO: 20, MACRO protein
MRNKKILKEDELLSETQQAAFHQIAMEPFEINVPKPKRRNGVNFSLAV
VVIYLILLTAGAGLLVVQVLNLQARLRVLEMYFLNDTLAAEDSPSFSL
LQSAHPGEHLAQGASRLQVLQAQLTWVRVSHEHLLQRVDNFTQNPGMF
RIKGEQGAPGLQGHKGAMGMPGAPGPPGPPAEKGAKGAMGRDGATGPS
GPQGPPGVKGEAGLQGPQGAPGKQGATGTPGPQGEKGSKGDGGLIGPK
GETGTKGEKGDLGLPGSKGDRGMKGDAGVMGPPGAQGSKGDFGRPGPP
GLAGFPGAKGDQGQPGLQGVPGPPGAVGHPGAKGEPGSAGSPGRAGLP
GSPGSPGATGLKGSKGDTGLQGQQGRKGESGVPGPAGVKGEQGSPGLA
GPKGAPGQAGQKGDQGVKGSSGEQGVKGEKGERGENSVSVRIVGSSNR
GRAEVYYSGTWGTICDDEWQNSDAIVFCRMLGYSKGRALYKVGAGTGQ
IWLDNVQCRGTESTLWSCTKNSWGHHDCSHEEDAGVECSV,
CDK6 planking

SEQ ID NO: 5

CCTCTGCCTATGTCTCTGCCTCTCTCTCTCTCTCTCTCTCTCTGTGAC
TATCATAAATAAATAAAAATTAAAAAAAAAAAAGATATTCAGTTCTGA
TCTGTGTCAGATTCACCGTGAAGTGTTCTCTTTTAAATAAATAAATAA
ATAAATAAATAAATAAGTAAGTAAGTAAATAAAGCGCTAAACATAACA
GGAAAGATTGGCCATACAGACTTCTTACAATTTAAAACGTCTTTTCAT
GGGACACCTGAATGGCTCAATGTTGGACATCCGACCCTCAATTTTGGC
TCAGGTTATGATCTCGGGGTCATGGGATCAAGTCCCACTAGACACAGT
CTGCTTGTTCTTCTCCCTCTGCTCCTCCTCAATTCTCTCTCTCTTTCT
CAAATGAATAAATAAAATCTTTAAAAAAATAAAACCTCTATTCATCAA
AATATAACATTAAGAGAATGAAAAGACNAGAAGTAATGTGGAATAAGA
CATTTTACATGGATAAATCATNCNAAGGACTATTTCTAGACCATATAA
ATATCTCTTANAAATTAATAAGNNNAAATTGTCTGACTCAATTATTTT
TAAGAGNAGGATAAAAGANTTGAATAGATTTTTTNCAAATGAAAATAT
CCCAATGGNCCAATGNCCATGAAAATATNNTCCNNCCNCNAAAGNTAT
CCGGAAAATGCNAGNNGGAAATTAAACN,
CDK6 CDNA

SEQ ID NO: 13

GGCTTCAGCCCTGCAGGGAAAGAAAAGTGCAATGATTCTGGACTGAGA
CGCGCTTGGGCAGAGGCTATGTAATCGTGTCTGTGTTGAGGACTTCGC
TTCGAGGAGGGAAGAGGAGGGATCGGCTCGCTCCTCCGGCGGCGGCGG
CGGCGGCGACTCTGCAGGCGGAGTTTCGCGGCGGCGGCACCAGGGTTA
CGCCAGCCCCGCGGGGAGGTCTCTCCATCCAGCTTCTGCAGCGGCGAA
AGCCCCAGCGCCCGAGCGCCTGAGCCGGCGGGGAGCAAGTAAAGCTAG
ACCGATCTCCGGGGAGCCCCGGAGTAGGCGAGCGGCGGCCGCCAGCTA
GTTGAGCGCACCCCCCGCCCGCCCCAGCGGCGCCGCGGCGGGCGGCGT
CCAGGCGGCATGGAGAAGGACGGCCTGTGCCGCGCTGACCAGCAGTAC
GAATGCGTGGCGGAGATCGGGGAGGGCGCCTATGGGAAGGTGTTCAAG
GCCCGCGACTTGAAGAACGGAGGCCGTTTCGTGGCGTTGAAGCGCGTG
CGGGTGCAGACCGGCGAGGAGGGCATGCCGCTCTCCACCATCCGCGAG
GTGGCGGTGCTGAGGCACCTGGAGACCTTCGAGCACCCCAACGTGGTC
AGGTTGTTTGATGTGTGCACAGTGTCACGAACAGACAGAGAAACCAAA
CTAACTTTAGTGTTTGAACATGTCGATCAAGACTTGACCACTTACTTG
GATAAAGTTCCAGAGCCTGGAGTGCCCACTGAAACCATAAAGGATATG
ATGTTTCAGCTTCTCCGAGGTCTGGACTTTCTTCATTCACACCGAGTA
GTGCATCGCGATCTAAAACCACAGAACATTCTGGTGACCAGCAGCGGA
CAAATAAAACTCGCTGACTTCGGCCTTGCCCGCATCTATAGTTTCCAG
ATGGCTCTAACCTCAGTGGTCGTCACGCTGTGGTACAGAGCACCCGAA
GTCTTGCTCCAGTCCAGCTACGCCACCCCCGTGGATCTCTGGAGTGTT
GGCTGCATATTTGCAGAAATGTTTCGTAGAAAGCCTCTTTTTCGTGGA
AGTTCAGATGTTGATCAACTAGGAAAAATCTTGGACGTGATTGGACTC
CCAGGAGAAGAAGACTGGCCTAGAGATGTTGCCCTTCCCAGGCAGGCT
TTTCATTCAAAATCTGCCCAACCAATTGAGAAGTTTGTAACAGATATC
GATGAACTAGGCAAAGACCTACTTCTGAAGTGTTTGACATTTAACCCA
GCCAAAAGAATATCTGCCTACAGTGCCCTGTCTCACCCATACTTCCAG
GACCTGGAAAGGTGCAAAGAAAACCTGGATTCCCACCTGCCGCCCAGC
CAGAACACCTCGGAGCTGAATACAGCCTGA1372GGCCTCAGCAGCCG
CCTTAAGCTGATCCTGCGGAGAACACCCTTGGTGGCTTATGGGTCCCC
CTCAGCAAGCCCTACAGAGCTGTGGAGGATTGCTATCTGGAGGCCTTC
CAGCTGCTGTCTTCTGGACAGGCTCTGCTTCTCCAAGGAAACCGCCTA
GTTTACTGTTTTGAAATCAATGCAAGAGTGATTGCAGCTTTATGTTCA
TTTGTTTGTTTGTTTGTCTGTTTGTTTCAAGAACCTGGAAAAATTCCA
GAAGAAGAGAAGCTGCTGACCAATTGTGCTGCCATTTGATTTTTCTAA
CCTTGAATGCTGCCAGTGTGGAGTGGGTAATCCAGGCACAGCTGAGTT
ATGATGTAATCTCTCTGCAGCTGCCGGGCCTGATTTGGTACTTTTGAG
TGTGTGTGTGCATGTGTGTGTGTGTGTGTGTGTGTGTGTGTGTGTATG
TGAGAGATTCTGTGATCTTTTAAAGTGTTACTTTTTGTAAACGACAAG
AATAATTCAATTTTAAAGACTCAAGGTGGTCAGTAAATAACAGGCATT
TGTTCACTGAAGGTGATTCACCAAAATAGTCTTCTCAAATTAGAAAGT
TAACCCCATGTCCTCAGCATTTCTTTTCTGGCCAAAAGCAGTAAATTT
GCTAGCAGTAAAAGATGAAGTTTTATACACACAGCAAAAAGGAGAAAA
AATTCTAGTATATTTTAAGAGATGTGCATGCATTCTATTTAGTCTTCA
GAATGCTGAATTTACTTGTTGTAAGTCTATTTTAACCTTCTGTATGAC
ATCATGCTTTATCATTTCTTTTGGAAAATAGCCTGTAAGCTTTTTATT
ACTTGCTATAGGTTTAGGGAGTGTACCTCAGATAGATTTTAAAAAAAA
GAATAGAAAGCCTTTATTTCCTGGTTTGAAATTCCTTTCTTCCCTTTT
TTTGTTGTTGTTATTGTTGTTTGTTGTTGTTATTTTGTTTTTGTTTTT
AGGAATTTGTCAGAAACTCTTTCCTGTTTTGGTTTGGAGAGTAGTTCT
CTCTAACTAGAGACAGGAGTGGCCTTGAAATTTTCCTCATCTATTACA
CTGTACTTTCTGCCACACACTGCCTTGTTGGCAAAGTATCCATCTTGT
CTATCTCCCGGCACTTCTGAAATATATTGCTACCATTGTATAACTAAT
AACAGATTGCTTAAGCTGTTCCCATGCACCACCTGTTTGCTTGCTTTC
AATGAACCTTTCATAAATTCGCAGTCTCAGCTTATGGTTTATGGCCTC
GATTCTGCAAACCTAACAGGGTCACATATGTTCTCTAATGCAGTCCTT
CTACCTGGTGTTTACTTTTGCTACCCAAATAATGAGTAGGATCTTGTT
TTCGTATACCCCCACCACTCCCATTGCTACCAACTGTCACCTTGTGCA
CTCCTTTTTTATAGAAGATATTTTCAGTGTCTTTACCTGAGGGTATGT
CTTTAGCTATGTTTTAGGGCCATACATTTACTCTATCAAATGATCTTT
TCTCCATCCCCCAGGCTGTGCTTATTTCTAGTGCCTTGTGCTCACTCC
TGCTCTCTACAGAGCCAGCCTGGCCTGGGCATTGTAAACAGCTTTTCC
TTTTTCTCTTACTGTTTTCTCTACAGTCCTTTATATTTCATACCATCT
CTGCCTTATAAGTGGTTTAGTGCTCAGTTGGCTCTAGTAACCAGAGGA
CACAGAAAGTATCTTTTGGAAAGTTTAGCCACCTGTGCTTTCTGACTC
AGAGTGCATGCAACAGTTAGATCATGCAACAGTTAGATTATGTTTAGG
GTTAGGATTTTCAAAGAATGGAGGTTGCTGCACTCAGAAAATAATTCA
GATCATGTTTATGCATTATTAAGTTGTACTGAATTCTTTGCAGCTTAA
TGTGATATATGACTATCTTGAACAAGAGAAAAAACTAGGAGATGTTTC
TCCTGAAGAGCTTTTGGGGTTGGGAACTATTCTTTTTTAATTGCTGTA
CTACTTAACATTGTTCTAATTCAGTAGCTTGAGGAACAGGAACATTGT
TTTCTAGAGCAAGATAATAAAGGAGATGGGCCATACAAATGTTTTCTA
CTTTCGTTGTGACAACATTGATTAGGTGTTGTCAGTACTATAAATGCT
TGAGATATAATGAATCCACAGCATTCAAGGTCAGGTCTACTCAAAGTC
TCACATGGAAAAGTGAGTTCTGCCTTTCCTTTGATCGAGGGTCAAAAT
ACAAAGACATTTTTGCTAGGGCCTACAAATTGAATTTAAAAACTCACT
GCACTGATTCATCTGAGCTTTTTGGTTAGTATTCATGGCTAGAGTGAA
CATAGCTTTAGTTTTTGCTGTTGTAAAAGTGTTTTCATAAGTTCACTC
AAGAAAAATGCAGCTGTTCTGAACTGGAATTTTTCAGCATTCTTTAGA
ATTTTAAATGAGTAGAGAGCTCAACTTTTATTCCTAGCATCTGCTTTT
GACTCATTTCTAGGCAGTGCTTATGAAGAAAAATTAAAGCACAAACAT
TCTGGCATTCAATCGTTGGCAGATTATCTTCTGATGACACAGAATGAA
AGGGCATCTCAGCCTCTCTGAACTTTGTAAAAATCTGTCCCCAGTTCT
TCCATCGGTGTAGTTGTTGCATTTGAGTGAATACTCTCTTGATTTATG
TATTTTATGTCCAGATTCGCCATTTCTGAAATCCAGATCCAACACAAG
CAGTCTTGCCGTTAGGGCATTTTGAAGCAGATAGTAGAGTAAGAACTT
AGTGACTACAGCTTATTCTTCTGTAACATATGGTTTCAAACATCTTTG
CCAAAAGCTAAGCAGTGGTGAACTGAAAAGGGCATATTGCCCCAAGGT
TACACTGAAGCAGCTCATAGCAAGTTAAAATATTGTGACAGATTTGAA
ATCATGTTTGAATTTCATAGTAGGACCAGTACAAGAATGTCCCTGCTA
GTTTCTGTTTGATGTTTGGTTCTGGCGGCTCAGGCATTTTGGGAACTG
TTGCACAGGGTGCAGTCAAAACAACCTACATATAAAAATTACATAAAA
GAACCTTGTCCATTTAGCTTTCATAAGAAATCCCATGGCAAAGAGTAA
TAAAAAGGACCTAATCTTAAAAATACAATTTCTAAGCACTTGTAAGAA
CCCAGTGGGTTGGAGCCTCCCACTTTGTCCCTCCTTTGAAGTGGATGG
GAACTCAAGGTGCAAAGAACCTGTTTTGGAAGAAAGCTTGGGGCCATT
TCAGCCCCCTGTATTCTCATGATTTTCTCTCAGGAAGCACACACTGTG
AATGGCAGACTTTTCATTTAGCCCCAGGTGACTTACTAAAAATAGTTG
AAAATTATTCACCTAAGAATAGAATCTCAGCATTGTGTTAAATAAAAA
TGAAAGCTTTAGAAGGCATGAGATGTTCCTATCTTAAATAAAGCATGT
TTCTTTTCTATAGAGAAATGTATAGTTTGACTCTCCAGAATGTACTAT
CCATCTTGATGAGAAAACTCTTAAATAGTACCAAACATTTTGAACTTT
AAATTATGTATTTAAAGTGAGTGTTTAAGAAACTGTAGCTGCTTCTTT
TACAAGTGGTGCCTATTAAAGTCAGTAATGGCCATTATTGTTCCATTG
TGGAAATTAAATTATGTAAGCTTCCTAATATCATAAACATATTAAAAT
TCTTCTAAAATATTGCTTTTCTTTTAAGTGACAATTTGACTATTCTTA
TGATAAGCACATGAGAGTGTCTTACATTTTCCAAAAGCAGGCTTTAAT
TGCATAGTTGAGTCTAGGAAAAAATAATGTTAAAAGTGAATATGCCAC
CATAATTACTTAATTATGTTAGTATAGAAACTACAGAATATTTACCCT
GGAAAGAAAATATTGGAATGTTATTATAAACTCTTAGATATTTATATA
ATTCAAAAGAATGCATGTTTCACATTGTGACAGATAAAGATGTATGAT
TTCTAAGGCTTTAAAAATTATTCATAAAACAGTGGGCAATAGATAAAG
GAAATTCTGGAGAAAATGAAGGTATTTAAAGGGTAGTTTCAAAGCTAT
ATATATTTTGAAGGATATATTCTTTATGAACAAATATATTGTAAAAAT
TTATACTAAGGTCATCTGGTAACTGTGGGATTAATATGGTCGAAAACA
AATGTTATGGAGAAGCTGTCCCAAGCAAACTAAATTACCTGTACTTTT
TTCCCATTTCAAGGGAAGAGGCAACCACATGAAGCAATACTTCTTACA
CATGCCTAAGAACGTTCATTGAAAAAATAAATTTTTAAAAGGCATGTG
TTTCCTATGCCACCAATACTTTTGAAAAATTGTGAACCTTACCCAAAA
CCATTTATCATGTCCATTAAGTATATTTGGGTATATAATTAGGAAGAT
ATTTACATGTTCCATCTCCACAGTGGAAAAACTTATTGAGGCTACCAA
AGTGTGCCAAGAAATGTAAGTCCTTAGAGTAATTAGAAATGCTGTTTT
CCTCAAAAGCATGAGAAACTAGCATTTTCATTTCTTATTTACTCCCTT
TCTATATCAATGCAATTCACAACCCAATTTTAATACATCCCTATATCT
CAAGCATTTCTATCTTGTACTTTTTCAGAAAATAAACCAAAAATAATC
CTTTGGTCTCTCTATCTTCTGACCTTTGTAAGCAACAGAAATGTAAAA
ACAGAAGGGGTCCAATTTTTACACGTTTTTTTCTCAAGTAGCCTTTCT
GGGGATTTTTATTTTCTTAATGAAGTGCCAATCAGCTTTTCAAAATGT
TTTCTATTTCTCAGCATTTCCAGGAAGTGATAACGTTTAGCTAAATGA
GTAGAAGTGGACTTCCTTCAACATATTGTTACCTTGTCTAGCCTTAGG
AAGAAAACAAGAGCCACCTGAAAATAAATACAGGCTCTTTTCGAGCAT
CTGCTGAAATACTGTTACAGCAATTTGAAGTTGATGTGGTAGGAAAGG
AAGGTGACTTTTCTTGCAAAAGTCTTTCTAAACATTCACACTGTCCTA
AGAGATGAGCTTTCTTGTTTTATTCCGGTATATTCCACAAGGTGGCAC
TTTTAGAGAAAAACAAATCTGATGAAGACTAAAGAGGTACTTCTAAAA
GAGATTTCATTCTAACTTTATTTTTCTGCGCATATTTAACTCTTTCCT
AGCACTTGTTTTTTGGGATGATTAATAGTCTCTATAATGTTCTGTAAC
TTCAATATTTTACTTGTTACCTAGGTTCTGAACAATTGTCTGCAAATA
AATTGTTCTTAAGGATGGATAATACACCCATTTTGATCATTTAAGTAA
AGAAAGCCTAGTCATTCATTCAGTCAAGAAAAAATTTTTGAAGTACCC
AGTTACCTTACTTTTCTAGATTAAAACAGGCTTAGTTACTAAAAAGGC
AGTCCTCATCTGTGAACAGGATAGTTTCGTTAGAAGTATAAAACTCCT
TTAGTGGCCCCAGTTAAAACACACATACCCTCTCTGCTGCTTTCAAAT
TCCCTAGCATGGTGGCCTTTCAACATTGATTAAATTTTAAAATCCTAA
TTTAAAGATCAGGTGAGCAAAATGAGTAGCACATCAGTAATTCAGTAG
ACAAAACTTTTGTCTGAAAAATTGCTGTATTGAAACAGAGCCCTAAAA
TACCAAAAGACCAGGTAATTTTAACATTTGTGGAATCACAAATGTAAA
TTCATAAGAAGCTCTAATTAAAAAAAAAAAGTCTGAAGTATATGAGCA
TAACAACTTAGGAGTGTGTCTACATACTTAACTTTTGAAGTTTTTTGG
CAACTTTATATACTTTTTTTAAATTTACAAGTCTACTTAAAGACTTCT
TATACCCCAAATGATTAAGTTAATTTTAGAGGTCACCTTTCTCACAGC
AGTGTCACTTGAAATTTAGTAGGGAAGGATATTGCAGTATTTTTCAGT
TTCCTTAGCACAGCACCACAGAAAGCAGCTTATTCCTTTTGAGTGGCA
GACACTCGACGGTGCCTGCCCAACTTTCCTCCTGAGTGGCAAGCAGAT
GAGTCTCAGTAATTCATACTGAACCAAAATGCCACATACACTAGGGGC
AGTCAGAAACTGGCTGAGAAATCCCCCGCCTCATTCGCCCCTCTGCTC
CCAGGAACTAGAGTCCAGTTAAAGCCCCTATGCGAAAGGCCGAATTCC
ACCCCAGGGTTTGTTATAACAGTGGCCAGTCTGAACCCCATTTGCTCG
TGCTCAAAACTTGATTCCCACTTGAAAGCCTTCCGGGCGCGCTGCCTC
GTTGCCCCGCCCCTTTGGCAGGAGAGAGGCAGTGGGCGAGGCCGGGCT
GGGGCCCCGCCTCCCACTCACCTGCCGGTGCCTGAAATTATGTGCGGC
CCCGCGGGCTGCTTTCCGAGGTCAGAGTGCCCTGCTGCTGTCTCAGAG
GCATCTGTTCTGCAAATCTTAGGAAGAAAAATGTCCCTAGTAGCAAAC
GGGTGTCTTCTGTGCATAAATAAGTACAACACAATTCTCCGAAAGTTC
GGGTAAAAAGAGATGCGGTAGCAGCTGCCCTGTGTGAAGCTGTCTACC
CCGCATCTCTCAGGCGCTAAGCTCAGTTTTTGTTTTTGTTTTTGTTTT
TTTAAAGAAAAGATGTATAATTGCAGGAATTTTTTTTTATTTTTTTAT
TTTCCATCATTCTATATATGTGATGGTGAAAGATATGCCTGGAAAAGT
TTTGTTTTGAAAAGTTTATTTTCTGCTTCGTCTTCAGTTGGCAAAAGC
TCTCAATTCTTTAGCTTCCAGTTTCTTTTCTCTCTTTTTCTTTGTTAG
GTAATTAAAGGTATGTAAACAAATTATCTCATGTAGCAGGGGATTTTC
ATGTTGAGAGGAATCTTCCGTGTGAGTTGTTTGGTCACACAAATAACC
CTTTCTCAATTTTAGGAGTTTGGATTGTCAAATGTAGGTTTTTCTCAA
AGGGGGCATATAACTACATATTGACTGCCAAGAACTATGACTGTAGCA
CTAATCAGCACACATAGAGCCACACAATTATTTAATTTCTAACTCTCT
GTGGTCCCTAGAAAAATTCCGTTGATGTGCTTAGGTTAAAGTTCTGAA
GATACCCGTTGTACCCTTACTTGAAAGTTTCTAATCTTAAGTTTTATG
AAATGCAATAATATGTATCAGCTAGCAATATTTCTGTGATCACCAACA
ACTCTCAGTTTGATCTTAAAGTCTGAATAATAAAACAAATCCCAGCAG
TAATACATTTCTTAAACCTCACAGTGCATGATATATCTTTTCATTCTG
ATCCTGTGTTTGCAAAAATATACACATGTATATCATAGTTCCTCACTT
TTTATTCATTTGTTTTCCTATTACCTGTAGTAAATATATTAGTTAGTA
CATGGAATTTATAGCATCAGCTACCCCCAGGAACAGCACCTGACAGGC
GGGGGATTTTTTTTCAAGTTGTTCTACATTTGCATAAATTATTTCTAT
TATTATTCATGTATGTTATTTATTTCTGAATCACACTAGTCCTGTGAA
AGTACAACTGAAGGCAGAAAGTGTTAGGATTTTGCATCTAATGTTCAT
TATCATGGTATTGATGGACCTAAGAAAATAAAAATTAGACTAAGCCCC
CAAATAAGCTGCATGCATTTGTAACATGATTAGTAGATTTGAATATAT
AGATGTAGTATTTTGGGTATCTAGGTGTTTTATCATTATGTAAAGGAA
TTAAAGTAAAGGACTTTGTAGTTGTTTTTATTAAATATGCATATAGTA
GAGTGCAAAAATATAGCAAAAATAAAAACTAAAGGTAGAAAAGCATTT
TAGATATGCCTTAATTTAGAAACTGTGCCAGGTGGCCCTCGGAATAGA
TGCCAGGCAGAGACCAGTGCCTGGGTGGTGCCTCCTCTTGTCTGCCCT
CATGAAGAAGCTTCCCTCACGTGATGTAGTGCCCTCGTAGGTGTCATG
TGGAGTAGTGGGAACAGGCAGTACTGTTGAGAGGAGAGCAGTGTGAGA
GTTTTTCTGTAGAAGCAGAACTGTCAGCTTGTGCCTTGAGGCTTCCAG
AACGTGTCAGATGGAGAAGTCCAAGTTTCCATGCTTCAGGCAACTTAG
CTGTGTACAGAAGCAATCCAGTGTGGTAATAAAAAGCAAGGATTGCCT
GTATAATTTATTATAAAATAAAAGGGATTTTAACAACCAACAATTCCC
AACACCTCAAAAGCTTGTTGCATTTTTTGGTATTTGAGGTTTTTATCT
GAAGGTTAAAGGGCAAGTGTTTGGTATAGAAGAGCAGTATGTGTTAAG
AAAAGAAAAATATTGGTCACGTAGAGTGCAAATTAGAACTAGAAAGTT
TTATACGATTATCATTTTGAGATGTGTTAAAGTAGGTTTTCACTGTAA
AATGTATTAGTGTTTCTGCATTGCCATAGGGCCTGGTTAAAACTTTCT
CTTAGGTTTCAGGAAGACTGTCACATACAGTAAGCTTTTTTCCTTCTG
ACTTATAATAGAAAATGTTTTGAAAGTAAAAAAAAAAAATCTAATTTG
GAAATTTGACTTGTTAGTTTCTGTGTTTGAAATCATGGTTCTAGAAAT
GTAGAAATTGTGTATATCAGATACTCATCTAGGCTGTGTGAACCAGCC
CAAGATGACCAACATCCCCACACCTCTACATCTCTGTCCCCTGTATCT
CTTCCTTTCTACCACTAAAGTGTTCCCTGCTACCATCCTGGCTTGTCC
ACATGGTGCTCTCCATCTTCCTCCACATCATGGACCACAGGTGTGCCT
GTCTAGGCCTGGCCACCACTCCCAACTTGACCTAGCCACATTCATCTA
GAGATGGTTCCTGATGCTGGGCACAGACTGTGCTCATGGCACCCATTA
GAAATGCCTCTAGCATCTTTGTATGCATCTTGATTTTTAAACCAAGTC
ATTGTACAGAGCATTCAGTTTTGGCTGTGGTACCAAGAGAAAAACTAA
TCAAGAATATAAACCACATTCCAGGCTGCTGTTTTCTCTCCATCTACA
GGCCACACTTTTACTGTATTTCTTCATACTTGAAATTCATTCTGCTAT
TTTCATATCAGGGTACAGACTTATAAGGGTGCATGTTCCTTAAAGGTG
CATAATTATTCTTATTCCGTTTGCTTATATTGCTACAGAATGCTCTGT
TTTGGTGCTTTGAGTTCTGCAGACCCAAGAAGCAGTGTGGAAATTCAC
TGCCTGGGACACAGTCTTATAAGAATGTTGGCAGGTGACTTTGTATCA
GATGTTGCTTCTCTTTTCTCTGTACACAGATTGAGAGTTACCACAGTG
GCCTGTCGGGTCCACCCTGTGGGTGCAGCACAGCTCTCTGAAAGCAAG
AACCTTCCTACCTATTCTAACGTTTTTGCCCTCTAAGAAAAATGGCCT
CAGGTATGGTATAGACATAGCAAGAGGGGAAGGGCTGTCTCACTCTAG
CAACCATCCCTCCATTACACACAGAAAGCCCTCTTGAAGCAAAAGAAG
AAGAAAGAAAGAAAGCTTATCTCTAAGGCTACTGTCTTCAGAATGCTC
TGAGCTGAATGCTCTTGCTCCTTTCCCAAGAGGCAGATGAAAATATAG
CCAGTTTATCTATACCCTTCCTATCTGAGGAGGAGAATAGAAAAGTAG
GGTAAATATGTAACGTAAAATATGTCATTCAAGGACCACCAAAACTTT
AAGTACCCTATCATTAAAAATCTGGTTTTAAAAGTAGCTCAAGTAAGG
GATGCTTTGTGACCCAGGGTTTCTGAAGTCAGATAGCCATTCTTACCT
GCCCCTTACTCTGACTTATTGGGAAAGGAGAACTGCAGTGGTGTTTCT
GTTGCAGTGGCAAAGGTAACATGTCAGAAAATTCAGAGGGTTGCATAC
CAATAATCCTTTGGAAACTGGATGTCTTACTGGGTGCTAGAATGAAAA
TGTAGGTATTTATTGTCAGATGATGAAGTTCATTGTTTTTTTCAAAAT
TGGTGTTGAAATATCACTGTCCAATGTGTTCACTTATGTGAAAGCTAA
ATTGAATGAGGCAAAAAGAGCAAATAGTTTGTATATTTGTAATACCTT
TTGTATTTCTTACAATAAAAATATTGGTAGCAAATAAAAATAATAAAA
ACAATAACTTTAAACTGCTTTCTGGAGATGAATTACTCTCCTGGCTAT
TTTCTTTTTTACTTTAATGTAAAATGAGTATAACTGTAGTGAGTAAAA
TTCATTAAATTCCAAGTTTTAGCAGAAAAAAAAAAAAAAAAAAAA
NO: 21, CDK6. Protein:
MEKDGLCRADQQYECVAEIGEGAYGKVFKARDLKNGGRFVALKRVRVQ
TGEEGMPLSTIREVAVLRHLETFEHPNVVRLFDVCTVSRTDRETKLTL
VFEHVDQDLTTYLDKVPEPGVPTETIKDMMFQLLRGLDFLHSHRVVHR
DLKPQNILVTSSGQIKLADFGLARIYSFQMALTSVVVTLWYRAPEVLL
QSSYATPVDLWSVGCIFAEMFRRKPLFRGSSDVDQLGKILDVIGLPGE
EDWPRDVALPRQAFHSKSAQPIEKFVTDIDELGKDLLLKCLTFNPAKR
ISAYSALSHPYFQDLERCKENLDSHLPPSQNTSELNTA
NO: 6, FLJ16046 flanking
TGATCTCCAGATTTACATATTCAGTTCCTACTTGACAACTCCCCTTGG
ATATTTCAAAGATATCTCAAATTCAAAGTGTCACACCTGTCACACACT
CTTCTGCTCTCTGCCCCTTCAACCTGATCCTCTCTTTTTTTNGACTCT
ATGAAAGGCATCNCCTTTCATTCTATTTAGCTAGAGACTANAAGGCAC
TCTAGCATTCTTTCTCTACCCCTTACCCAATTGATTACCTAATCCCAT
GGATTTCACCTCCTTAAATATCTCTGTCATCTCTTGCTTCCCTTGTCC
CACTTTATCTTCACCACCTCCACCTCCCGCCATCCAGAGAAATTAGTC
ATCCAGCTAGTTTCCTTATATTTACCTTTATACTCCTTTCCTGCATTA
GNCATATGAAAGCCACAATGATTTCTAACAAGATACTAATCTGATATC
CTGTTAAACTCCTTCNTAAAAAACTTTAGTGGCTTACCTTCAGTCTTA
AGATAGAAAATATAACTTCTAAGAAGGACCCACATGGNTCCTCAAGGA
CTAGTTCTCCTGACCTCTCCATTCTCATCACACAGGACTTGCCCCCTT
GCTGTCTTCTCTTCAGTCCTGCTTNTGNNTCCCCCAGAAATTTTGTGT
ATGCCAGGCTCCTACATGCCAAAGAGCATTTGCAATGCTGTTCCCTCT
GTTTTAGAAAANCTTATA
NO: 14, FLJ16046 cDNA
GATACAGATCAGATGGTGACTGAATAGAAGCTGCCCCAGTCCTGGGCT
CATGATGTACGCACCTGTTGAATTTTCAGAAGCTGAATTCTCACGAGC
TGAATATCAAAGAAAGCAGCAATTTTGGGACTCAGTACGGCTAGCTCT
TTTCACATTAGCAATTGTAGCAATCATAGGAATTGCAATTGGTATTGT
TACTCATTTTGTTGTTGAGGATGATAAGTCTTTCTATTACCTTGCCTC
TTTTAAAGTCACAAATATCAAATATAAAGAAAATTATGGCATAAGATC
TTCAAGAGAGTTTATAGAAAGGAGTCATCAGATTGAAAGAATGATGTC
TAGGATATTTCGACATTCTTCTGTAGGCGGTCGATTTATCAAATCTCA
TGTTATCAAATTAAGTCCAGATGAACAAGGTGTGGATATTCTTATAGT
GCTCATATTTCGATACCCATCTACTGATAGTGCTGAACAAATCAAGAA
AAAAATTGAAAAGGCTTTATATCAAAGTTTGAAGACCAAACAATTGTC
TTTGACCTTAAACAAACCATCATTTAGACTCACACCTATTGACAGCAA
AAAGATGAGGAATCTTCTCAACAGTCGCTGTGGAATAAGGATGACATC
TTCAAACATGCCATTACCAGCATCCTCTTCTACTCAAAGAATTGTCCA
AGGAAGGGAAACAGCTATGGAAGGGGAATGGCCATGGCAGGCCAGCCT
CCAGCTCATAGGGTCAGGCCATCAGTGTGGAGCCAGCCTCATCAGTAA
CACATGGCTGCTCACAGCAGCTCACTGCTTTTGGAAAAATAAAGACCC
AACTCAATGGATTGCTACTTTTGGTGCAACTATAACACCACCCGCAGT
GAAACGAAATGTGAGGAAAATTATTCTTCATGAGAATTACCATAGAGA
AACAAATGAAAATGACATTGCTTTGGTTCAGCTCTCTACTGGAGTTGA
GTTTTCAAATATAGTCCAGAGAGTTTGCCTCCCAGACTCATCTATAAA
GTTGCCACCTAAAACAAGTGTGTTCGTCACAGGATTTGGATCCATTGT
AGATGATGGACCTATACAAAATACACTTCGGCAAGCCAGAGTGGAAAC
CATAAGCACTGATGTGTGTAACAGAAAGGATGTGTATGATGGCCTGAT
AACTCCAGGAATGTTATGTGCTGGATTCATGGAAGGAAAAATAGATGC
ATGTAAGGGAGATTCTGGTGGACCTCTGGTTTATGATAATCATGACAT
CTGGTACATTGTGGGTATAGTAAGTTGGGGACAATCATGTGCGCTTCC
CAAAAAACCTGGAGTCTACACCAGAGTAACTAAGTATCGAGATTGGAT
TGCCTCAAAGACCGGTATGTAGTGTGGATTGTCCATGAGTTATACACA
TGGCACACAGAGCTGATACTCCTGCGTATTTTGTATTGTTTAAATTCA
TTTACTTTGGATTAGTGCTTTTGCTAGATGTCAAGAAGCCCTTCAGAC
CCAGACAAATCTAATATCCTGAGGTGGCCTTTACATACGTAGGACCAA
ACCCTCTCTACCATGAGGGAAGAAGACACAGCAAATGACAGACAGCAC
CTATTCCTTACTCACAAGGGAAACTGCTTGTGATACTTCCTAATAAGA
TAAATGAGTGGTTTCCCTCAATTGAAGACAGGAACATCATTTTCCACA
GGATATGAAGAGCTGCCAGTAATGCCAAAATCTTACCTCATATAATAC
CTGGAGCATGTGAGATTCTTCTAGTGAAAAAGAACAGTCTTCCCTGAA
GACTCAGGGCTTCAACATTCTAGAACTGATAAGTGGACCTTCAGTGTG
CAAGAATGGAGAAGCATGGGATTTGCATTATGACTTGAACTGGGCTTA
TATCTAATAATACAGAGCACTATCACTAACCTCAACAGTTGACATTTT
AAAAGTTTTTAAATGTATCTGAACTTGCTGTTAACACAGTGTTATAAC
TCAAGCACTAGCTTCAGGAAGCATGTTGTGTTGTTAAGAAGCTTTTCT
GATTTATTCTTTAACAGCATCTTGCCATCTATATGTTAGTAGCAGTTG
GCCCAGAAAGGAC
NO: 22, FLJ16046 protein
MMYAPVEFSEAEFSRAEYQRKQQFWDSVRLALFTLAIVAIIGIAIGIV
THFVVEDDKSFYYLASFKVTNIKYKENYGIRSSREFIERSHQIERMMS
RIFRHSSVGGRFIKSHVIKLSPDEQGVDILIVLIFRYPSTDSAEQIKK
KIEKALYQSLKTKQLSLTLNKPSFRLTPIDSKKMRNLLNSRCGIRMTS
SNMPLPASSSTQRIVQGRETAMEGEWPWQASLQLIGSGHQCGASLISN
TWLLTAAHCFWKNKDPTQWIATFGATITPPAVKRNVRKIILHENYHRE
TNENDIALVQLSTGVEFSNIVQRVCLPDSSIKLPPKTSVFVTGFGSIV
DDGPIQNTLRQARVETISTDVCNRKDVYDGLITPGMLCAGFMEGKIDA
CKGDSGGPLVYDNHDIWYIVGIVSWGQSCALPKKPGVYTRVTKYRDWI
ASKTGM,
PCSK flanking

SEQ ID NO: 7

TGTTCTATGTATTATATAGATGAAATATCTTTCTTCTATCTTCCCTGA
GGACACCATATGAGATAACAGAATTTATATCCTGGTCTCTGTTTTAGT
TCTTGGCACANAGCTCCTGAGAACCTTGTCATTTCCTGATTGGGAAGA
GCAATAGGAGGATCTTTTGTTATAATATTTGCCTTTGACCCTGTTCCT
GACTCAGTACTAACATCCTTGTAAATTCCTAAGTGATAAGAGCACTAG
GAACATCCTTTGTTCTACGAAGGGGACTTGGGGTGGGCTCCTGGATGG
GGGCTGGTCACCAAAAGGACCAAGCTACGATTANAAACTTGGAATTTT
CAGCCCTGTCCCCCACTTCTCTANAGAGGGGAGAACAATNAAGTCCNT
TACTGATCATACCTACCTGAGGAAGCCTCCTTAAAATCNCAATAGNNA
TGAGGATCTGGNGAGATTCCNAANTGNGCNAACNCATNCNNTNCCNNG
AGGGTGNNNNACCCNNNCNCTGCCNGGNCAGANCCNCCTNGTNTTGNN
ANCTNCCCNTACTTAACCNTTCCNNGGAANTCNTCAGAGT,
PCSK6 cDNA

SEQ ID NO: 15

TCGCGGGCCGAGGACGCCTCTGGGGCGGCACCGCGTCCCGAGAGCCCC
AGAAGTCGGCGGGGAAGTTTCCCCGGTGGGGGGCGTTTCGGGCCTCCC
GGACGGCTCTCGGCCCCGGAGCCCGGTCGCAGGAGCGCGGGCCCGGGG
GCGGGAACGCGCCGCGGCCGCCTCCTCCTCCCCGGCTCCCGCCCGCGG
CGGTGTTGGCGGCGGCGGTGGCGGCGGCGGCGGCGCTTCCCCGGCGCG
GAGCGGCTTTAAAAGGCGGCACTCCACCCCCCGGCGCACTCGCAGCTC
GGGCGCCGCGCGAGCCTGTCGCCGCTATGCCTCCGCGCGCGCCGCCTG
CGCCCGGGCCCCGGCCGCCGCCCCGGGCCGCCGCCGCCACCGACACCG
CCGCGGGCGCGGGGGGCGCGGGGGGCGCGGGGGGCGCCGGCGGGCCCG
GGTTCCGGCCGCTCGCGCCGCGTCCCTGGCGCTGGCTGCTGCTGCTGG
CGCTGCCTGCCGCCTGCTCCGCGCCCCCGCCGCGCCCCGTCTACACCA
ACCACTGGGCGGTGCAAGTGCTGGGCGGCCCGGCCGAGGCGGACCGCG
TGGCGGCGGCGCACGGGTACCTCAACTTGGGCCAGATTGGAAACCTGG
AAGATTACTACCATTTTTATCACAGCAAAACCTTTAAAAGATCAACCT
TGAGTAGCAGAGGCCCTCACACCTTCCTCAGAATGGACCCCCAGGTGA
AATGGCTCCAGCAACAGGAAGTGAAACGAAGGGTGAAGAGACAGGTGC
GAAGTGACCCGCAGGCCCTTTACTTCAACGACCCCATTTGGTCCAACA
TGTGGTACCTGCATTGTGGCGACAAGAACAGTCGCTGCCGGTCGGAAA
TGAATGTCCAGGCAGCGTGGAAGAGGGGCTACACAGGAAAAAACGTGG
TGGTCACCATCCTTGATGATGGCATAGAGAGAAATCACCCTGACCTGG
CCCCAAATTATGATTCCTACGCCAGCTACGACGTGAACGGCAATGATT
ATGACCCATCTCCACGATATGATGCCAGCAATGAAAATAAACACGGCA
CTCGTTGTGCGGGAGAAGTTGCTGCTTCAGCAAACAATTCCTACTGCA
TCGTGGGCATAGCGTACAATGCCAAAATAGGAGGCATCCGCATGCTGG
ACGGCGATGTCACAGATGTGGTCGAGGCAAAGTCGCTGGGCATCAGAC
CCAACTACATCGACATTTACAGTGCCAGCTGGGGGCCGGACGACGACG
GCAAGACGGTGGACGGGCCCGGCCGACTGGCTAAGCAGGCTTTCGAGT
ATGGCATTAAAAAGGGCCGGCAGGGCCTGGGCTCCATTTTCGTCTGGG
CATCTGGGAATGGCGGGAGAGAGGGGGACTACTGCTCGTGCGATGGCT
ACACCAACAGCATCTACACCATCTCCGTCAGCAGCGCCACCGAGAATG
GCTACAAGCCCTGGTACCTGGAAGAGTGTGCCTCCACCCTGGCCACCA
CCTACAGCAGTGGGGCCTTTTATGAGCGAAAAATCGTCACCACGGATC
TGCGTCAGCGCTGTACCGATGGCCACACTGGGACCTCAGTCTCTGCCC
CCATGGTGGCGGGCATCATCGCCTTGGCTCTAGAAGCAAACAGCCAGT
TAACCTGGAGGGACGTCCAGCACCTGCTAGTGAAGACATCCCGGCCGG
CCCACCTGAAAGCGAGCGACTGGAAAGTGAACGGCGCGGGTCATAAAG
TTAGCCATTTCTATGGATTTGGTTTGGTGGACGCAGAAGCTCTCGTTG
TGGAGGCAAAGAAGTGGACAGCAGTGCCATCGCAGCACATGTGTGTGG
CCGCCTCGGACAAGAGACCCAGGAGCATCCCCTTAGTGCAGGTGCTGC
GGACTACGGCCCTGACCAGCGCCTGCGCGGAGCACTCGGACCAGCGGG
TGGTCTACTTGGAGCACGTGGTGGTTCGCACCTCCATCTCACACCCAC
GCCGAGGAGACCTCCAGATCTACCTGGTTTCTCCCTCGGGAACCAAGT
CTCAACTTCTGGCAAAGAGGTTGCTGGATCTTTCCAATGAAGGGTTTA
CAAACTGGGAATTCATGACTGTCCACTGCTGGGGAGAAAAGGCTGAAG
GGCAGTGGACCTTGGAAATCCAAGATCTGCCATCCCAGGTCCGCAACC
CGGAGAAGCAAGGGAAGTTGAAAGAATGGAGCCTCATACTGTATGGCA
CAGCAGAGCACCCGTACCACACCTTCAGTGCCCATCAGTCCCGCTCGC
GGATGCTGGAGCTCTCAGCCCCAGAGCTGGAGCCACCCAAGGCTGCCC
TGTCACCCTCCCAGGTGGAAGTTCCTGAAGATGAGGAAGATTACACAG
GTGTGTGCCATCCGGAGTGTGGTGACAAAGGCTGTGATGGCCCCAATG
CAGACCAGTGCTTGAACTGCGTCCACTTCAGCCTGGGGAGTGTCAAGA
CCAGCAGGAAGTGCGTGAGTGTGTGCCCCTTGGGCTACTTTGGGGACA
CAGCAGCAAGACGCTGTCGCCGGTGCCACAAGGGGTGTGAGACCTGCT
CCAGCAGAGCTGCGACGCAGTGCCTGTCTTGCCGCCGCGGGTTCTATC
ACCACCAGGAGATGAACACCTGTGTGACCCTCTGTCCTGCAGGATTTT
ATGCTGATGAAAGTCAGAAAAATTGCCTTAAATGCCACCCAAGCTGTA
AAAAGTGCGTGGATGAACCTGAGAAATGTACTGTCTGTAAAGAAGGAT
TCAGCCTTGCACGGGGCAGCTGCATTCCTGACTGTGAGCCAGGCACCT
ACTTTGACTCAGAGCTGATCAGATGTGGGGAATGCCATCACACCTGCG
GAACCTGCGTGGGGCCAGGCAGAGAAGAGTGCATTCACTGTGCGAAAA
ACTTCCACTTCCACGACTGGAAGTGTGTGCCAGCCTGTGGTGAGGGCT
TCTACCCAGAAGAGATGCCGGGCTTGCCCCACAAAGTGTGTCGAAGGT
GTGACGAGAACTGCTTGAGCTGTGCAGGCTCCAGCAGGAACTGTAGCA
GGTGTAAGACGGGCTTCACACAGCTGGGGACCTCCTGCATCACCAACC
ACACGTGCAGCAACGCTGACGAGACATTCTGCGAGATGGTGAAGTCCA
ACCGGCTGTGCGAACGGAAGCTCTTCATTCAGTTCTGCTGCCGCACGT
GCCTCCTGGCCGGGTAAGGGTGCCTAGCTGCCCACAGAGGGCAGGCAC
TCCCATCCATCCATCCGTCCACCTTCCTCCAGACTGTCGGCCAGAGTC
TGTTTCAGGAGCGGCGCCCTGCACCTGACAGCTTTATCTCCCCAGGAG
CAGCATCTCTGAGCACCCAAGCCAGGTGGGTGGTGGCTCTTAAGGAGG
TGTTCCTAAAATGGTGATATCCTCTCAAATGCTGCTTGTTGGCTCCAG
TCTTCCGACAAACTAACAGGAACAAAATGAATTCTGGGAATCCACAGC
TCTGGCTTTGGAGCAGCTTCTGGGACCATAAGTTTACTGAATCTTCAA
GACCAAAGCAGAAAAGAAAGGCGCTTGGCATCACACATCACTCTTCTC
CCCGTGCTTTTCTGCGGCTGTGTAGTAAATCTCCCCGGCCCAGCTGGC
GAACCCTGGGCCATCCTCACATGTGACAAAGGGCCAGCAGTCTACCTG
CTCGTTGCCTGCCACTGAGCAGTCTGGGGACGGTTTGGTCAGACTATA
AATAAGATAGGTTTGAGGGCATAAAATGTATGACCACTGGGGCCGGAG
TATCTATTTCTACATAGTCAGCTACTTCTGAAACTGCAGCAGTGGCTT
AGAAAGTCCAATTCCAAAGCCAGACCAGAAGATTCTATCCCCCGCAGC
GCTCTCCTTTGAGCAAGCCGAGCTCTCCTTGTTACCGTGTTCTGTCTG
TGTCTTCAGGAGTCTCATGGCCTGAACGACCACCTCGACCTGATGCAG
AGCCTTCTGAGGAGAGGCAACAGGAGGCATTCTGTGGCCAGCCAAAAG
GTACCCCGATGGCCAAGCAATTCCTCTGAACAAAATGTAAAGCCAGCC
ATGCATTGTTAATCATCCATCACTTCCCATTTTATGGAATTGCTTTTA
AAATACATTTGGCCTCTGCCCTTCAGAAGACTCGTTTTTAAGGTGGAA
ACTCCTGTGTCTGTGTATATTACAAGCCTACATGACACAGTTGGATTT
ATTCTGCCAAACCTGTGTAGGCATTTTATAAGCTACATGTTCTAATTT
TTACCGATGTTAATTATTTTGACAAATATTTCATATATTTTCATTGAA
ATGCACAGATCTGCTTGATCAATTCCCTTGAATAGGGAAGTAACATTT
GCCTTAAATTTTTTCGACCTCGTCTTTCTCCATATTGTCCTGCTCCCC
TGTTTGACGACAGTGCATTTGCCTTGTCACCTGTGAGCTGGAGAGAAC
CCAGATGTTGTTTATTGAATCTACAACTCTGAAAGAGAAATCAATGAA
GCAAGTACAATGTTAACCCTAAATTAATAAAAGAGTTAACATCCCATG
GC,
PCSK6 Protein

SEQ ID NO: 23

MPPRAPPAPGPRPPPRAAAATDTAAGAGGAGGAGGAGGPGFRPLAPRP
WRWLLLLALPAACSAPPPRPVYTNHWAVQVLGGPAEADRVAAAHGYLN
LGQIGNLEDYYHFYHSKTFKRSTLSSRGPHTFLRMDPQVKWLQQQEVK
RRVKRQVRSDPQALYFNDPIWSNMWYLHCGDKNSRCRSEMNVQAAWKR
GYTGKNVVVTILDDGIERNHPDLAPNYDSYASYDVNGNDYDPSPRYDA
SNENKHGTRCAGEVAASANNSYCIVGIAYNAKIGGIRMLDGDVTDVVE
AKSLGIRPNYIDIYSASWGPDDDGKTVDGPGRLAKQAFEYGIKKGRQG
LGSIFVWASGNGGREGDYCSCDGYTNSIYTISVSSATENGYKPWYLEE
CASTLATTYSSGAFYERKIVTTDLRQRCTDGHTGTSVSAPMVAGIIAL
ALEANSQLTWRDVQHLLVKTSRPAHLKASDWKVNGAGHKVSHFYGFGL
VDAEALVVEAKKWTAVPSQHMCVAASDKRPRSIPLVQVLRTTALTSAC
AEHSDQRVVYLEHVVVRTSISHPRRGDLQIYLVSPSGTKSQLLAKRLL
DLSNEGFTNWEFMTVHCWGEKAEGQWTLEIQDLPSQVRNPEKQGKLKE
WSLILYGTAEHPYHTFSAHQSRSRMLELSAPELEPPKAALSPSQVEVP
EDEEDYTGVCHPECGDKGCDGPNADQCLNCVHFSLGSVKTSRKCVSVC
PLGYFGDTAARRCRRCHKGCETCSSRAATQCLSCRRGFYHHQEMNTCV
TLCPAGFYADESQKNCLKCHPSCKKCVDEPEKCTVCKEGFSLARGSCI
PDCEPGTYFDSELIRCGECHHTCGTCVGPGREECIHCAKNFHFHDWKC
VPACGEGFYPEEMPGLPHKVCRRCDENCLSCAGSSRNCSRCKTGFTQL
GTSCITNHTCSNADETFCEMVKSNRLCERKLFIQFCCRTCLLAG,
PTGDR flanking

SEQ ID NO: 8

GGTGCCTTAGACATTACAGGCGGGGCACCATGGGTGGCATCAGTGGTT
GAGATGACTGCCTTTGACTCAGGGTGTGACCCATGGGGTCCTGGGATC
AAGTCCTGCATCCGGCTCCCTGCAGGGAGCCCACTTCTCCCTCTTCCT
AGGTCTCTGCCTCTCTCCTTATATCTCTCATGAATAAATAAATAAAAA
TCTTTAAAAAAAATTAGAGGCATTATGGATGGCACGTGATGTGATTAG
CATTGGATTGACAAATTGACAAATTGAATTTAAGTAAAAAAAAATACA
GGNAAAAATGCTACTGGGAGGGGTGCCTGGGTCGCTCTGTTGGTTAAA
ACTTTGCCTTTGGCTCAGGTCATGATCTCAGGGTTCTGNGNATTGAGC
CCCACCTTAGGCTCTGCTTGTTTCTCTGCCCCTCCCCCTGCTNNNNTT
TCTATCGAATAAANAAAANCCTTAAAAAAAAATGCTATTGGGAGTTAT
TTGATTACCTACAAGTGAAAAGATNTGACAGTCGGAGATCANAAAAAC
ATTATGTCTATTACNTATTTTANCTTTTTTTTTTTTT,
PTCGR cDNA

SEQ ID NO: 16

CGCCCGAGCCGCGCGCGGAGCTGCCGGGGGCTCCTTAGCACCCGGGCG
CCGGGGCCCTCGCCCTTCCGCAGCCTTCACTCCAGCCCTCTGCTCCCG
CACGCCATGAAGTCGCCGTTCTACCGCTGCCAGAACACCACCTCTGTG
GAAAAAGGCAACTCGGCGGTGATGGGCGGGGTGCTCTTCAGCACCGGC
CTCCTGGGCAACCTGCTGGCCCTGGGGCTGCTGGCGCGCTCGGGGCTG
GGGTGGTGCTCGCGGCGTCCACTGCGCCCGCTGCCCTCGGTCTTCTAC
ATGCTGGTGTGTGGCCTGACGGTCACCGACTTGCTGGGCAAGTGCCTC
CTAAGCCCGGTGGTGCTGGCTGCCTACGCTCAGAACCGGAGTCTGCGG
GTGCTTGCGCCCGCATTGGACAACTCGTTGTGCCAAGCCTTCGCCTTC
TTCATGTCCTTCTTTGGGCTCTCCTCGACACTGCAACTCCTGGCCATG
GCACTGGAGTGCTGGCTCTCCCTAGGGCACCCTTTCTTCTACCGACGG
CACATCACCCTGCGCCTGGGCGCACTGGTGGCCCCGGTGGTGAGCGCC
TTCTCCCTGGCTTTCTGCGCGCTACCTTTCATGGGCTTCGGGAAGTTC
GTGCAGTACTGCCCCGGCACCTGGTGCTTTATCCAGATGGTCCACGAG
GAGGGCTCGCTGTCGGTGCTGGGGTACTCTGTGCTCTACTCCAGCCTC
ATGGCGCTGCTGGTCCTCGCCACCGTGCTGTGCAACCTCGGCGCCATG
CGCAACCTCTATGCGATGCACCGGCGGCTGCAGCGGCACCCGCGCTCC
TGCACCAGGGACTGTGCCGAGCCGCGCGCGGACGGGAGGGAAGCGTCC
CCTCAGCCCCTGGAGGAGCTGGATCACCTCCTGCTGCTGGCGCTGATG
ACCGTGCTCTTCACTATGTGTTCTCTGCCCGTAATTTATCGCGCTTAC
TATGGAGCATTTAAGGATGTCAAGGAGAAAAACAGGACCTCTGAAGAA
GCAGAAGACCTCCGAGCCTTGCGATTTCTATCTGTGATTTCAATTGTG
GACCCTTGGATTTTTATCATTTTCAGATCTCCAGTATTTCGGATATTT
TTTCACAAGATTTTCATTAGACCTCTTAGGTACAGGAGCCGGTGCAGC
AATTCCACTAACATGGAATCCAGTCTGTGA1182CAGTGTTTTTCACT
CTGTGGTAAGCTGAGGAATATGTCACATTTTCAGTCAAAGAACCATGA
TTAAAAAAAAAAAGACAACTTACAATTTAAATCCTTAAAAGTTACCTC
CCATAACAAAAGCATGTATATGTATTTTCAAAAGTATTTGATATCTTA
ACAATGTGTTACCATTCTATAGTCATGAACCCCTTCAGTGCATTTTCA
TTTTTTTATTAACAGCAACTAAAATTTTATATATTGTAACCAGTGTTA
AAAGTCTTAAAAAACAATGGTATTAATTGTCCCTACATTTGTGCTTGG
TGGCCCTATTTTTTTTTTTTAGAGAGGCCTTGAGACATACAGGTCTTT
TAAAATACAGTAGAAACACCACTGTTTACGATTATACGATGGACATTC
ATAAAAAGCATAATTTCTTACCCTATTCATTTTTTGGTGAAACCTGAT
TCATTGATTTTATATCATTGCCGATGTTTAGTTCATTTCTTTGCCAAT
TGATCTAAGCATAGCCTGAATTATGATGTTCCTCAGAGAAGTGAGGTG
GGAAATATGACCAGGTCAGGCAGTTGGAGGGGCTTCCCCAGCCACCAT
CGGGGAGTACTTGCTGCCTCAGGTGGAGACCTGAAGCTGTAACTAGAT
GCAGAGCAAGATATGACTATAGCCCACAACCCAAAGAAGCAAAAATTC
GTTTTTATCTTTTGAAATCCAGTTTCTTTTGTATTGAGTCAAGGGTGT
CAGTAGGAATCAAAAGTTGGGGGTGGGTTGCAAAATGTTCTTTCAGTT
TTTAGAACCTCCATTTTATAAAAGAATTATCCTATCAATGGATTCTTT
AGTGGAAGGATTTATGCTTCTTTGAAAACCAGTGTGTGACTCACTGTA
GAGCCATGTTTACTGTTTGACTGTGTGGCACAGGGGGGCATTTGGCAC
AGCAAAAAGCCCACCCAGGACTTAGCCTCAGTTGACGATAGTAACAAT
GGCCTTAACATCTACCTTAACAGCTACCTATTACAGCCGTATTCTGCT
GTCCGTGGAGACGGTAAGATCTTAGGTTCCAAGATTTTACTTCAAATT
ACACCTTCAAAACTGGAGCAGCATATAGCCGAAAAGGAGCACAACTGA
GCACTTTAATAGTAATTTAAAAGTTTTCAAGGGTCAGCAATATGATGA
CTGAAAGGGAAAAGTGGAGGAAACGCAGCTGCAACTGAAGCGGAGACT
CTAAACCCAGCTTGCAGGTAAGAGCTTTCACCTTTGGTAAAAGAACAG
CTGGGGAGGTTCAAGGGGTTTCAGCATCTCTGGAGTTCCTTTGTATCT
GACAATCTCAGGACTCCAAGGTGCAAAGCCTGCTGCATTTGCGTGATC
TCAAGACCTCCAGCCAGAAGTCCCTTCCAAATATAAGAGTACTCATGT
TTATTTATTTCCAACTGAGCAGCAACCTCCTTTGTTTCACTTATGTTT
TTTCCAGTATCTGAGATAATATAAAGCTGGGTAATTTTTTATGTAATT
TTTTGGTATAGCAAAACTGTGAAAAAGCCAAATTAGGCATACAAGGAG
TATGATTTAACAGTATGACATGATGAAAAAAATACAGTTGTTTTTGAA
ATTTAACTTTTGTTTGTACCTTCAATGTGTAAGTACATGCATGTTTTA
TTGTCAGAGGAAGAACATGTTTTTTGTATTCTTTTTTTGGAGAGGTGT
GTTAGGATAATTGTCCAGTTAATTTGAAAAGGCCCCAGATGAATCAAT
AAATATAATTTTATAGTAAAAAAAAAAAAAAAAAAAAAAAAA,
PTCGR Protein

SEQ ID NO: 24

MKSPFYRCQNTTSVEKGNSAVMGGVLFSTGLLGNLLALGLLARSGLGW
CSRRPLRPLPSVFYMLVCGLTVTDLLGKCLLSPVVLAAYAQNRSLRVL
APALDNSLCQAFAFFMSFFGLSSTLQLLAMALECWLSLGHPFFYRRHI
TLRLGALVAPVVSAFSLAFCALPFMGFGKFVQYCPGTWCFIQMVHEEG
SLSVLGYSVLYSSLMALLVLATVLCNLGAMRNLYAMHRRLQRHPRSCT
RDCAEPRADGREASPQPLEELDHLLLLALMTVLFTMCSLPVIYRAYYG
AFKDVKEKNRTSEEAEDLRALRFLSVISIVDPWIFIIFRSPVFRIFFH
KIFIRPLRYRSRCSNSTNMESSL

### Preferred Embodiments

One aspect of the invention relates to a method for preventing or treating influenza in a subject. In one embodiment, the method comprises the step of modulating the expression of one or more influenza resistant genes of Table 3 in said subject.

In a related embodiment, the method comprises over-expressing a polypeptide comprising a sequence recited in any one of SEQ ID NOS: 18, 19 and 22, or a variant thereof, in the subject.

In another related embodiment, the method comprises inhibiting expression of a polypeptide comprising a sequence recited in any one of SEQ ID NOS: 17, 20, 21, 23 and 24, or a variant thereof, in the subject.

A second aspect of the invention relates to a pharmaceutical composition for preventing or treating influenza in a subject, said composition comprising a pharmaceutically acceptable carrier and a non-carrier component selected from the group consisting of:

(a) a polynucleotide comprising a sequence recited in any one of SEQ ID NOS:9-16, or a variant thereof,

(b) a polypeptide comprising an amino acid sequence recited in any one of SEQ ID NOS: 17-24, or a variant thereof,

(c) an agent capable of modulating the expression level of the polynucleotide of (a);

(d) an agent capable of modulating the expression level of the polypeptide of (b); and

(e) an agent capable of modulating the activity of the polypeptide of (b).

In a related embodiment, the pharmaceutical composition further comprises a pharmaceutically acceptable delivery vehicle.

A third aspect of the present invention relates to a method for preventing or treating influenza in a subject, comprising the step of introducing into the subject an effective amount of the pharmaceutical composition described above.

A fourth aspect of the present invention relates to a method for identifying an agent capable of binding to an influenza-related polypeptide, said method comprising:

contacting a polypeptide encoded by a gene listed in Table 3 or a homolog thereof with a candidate agent; and

determining a binding affinity of said candidate agent to said polypeptide.

In a related embodiment, the polypeptide or the candidate agent contains a label.

A fifth aspect of the present invention relates to a method for identifying an agent capable of modulating an activity of an influenza-related polypeptide, said method comprising the steps of:

contacting a polypeptide encoded by a gene listed in Table 3 or a homolog thereof,

determining the activity of said polypeptide in the presence of said candidate agent;

determining the activity of said polypeptide in the absence of said candidate agent; and

determining whether said candidate agent affects the activity of said polypeptide.

A sixth aspect of the present invention relates to a biochip comprising at least one of:

(a) a polynucleotide comprising a sequence that hybridizes to a gene listed in Table 3 or a homolog thereof;

(b) a polypeptide comprising at least a portion of a sequence encoded by a gene listed in Table 3.

What is claimed is:

1. A method for enhancing the resistance of a mammal to infection by an influenza virus, comprising altering the level of an influenza resistance gene product in said individual so as to increase the resistance of said individual to infection by an influenza virus.

2. The method of claim 1, wherein said step of altering the level of an influenza resistance gene comprises causing an influenza resistance gene the expression of which into a gene product improves the resistance of said individual to be overexpressed.

3. The method of claim 2, wherein said method comprises inserting, into the cells of said mammal, a vector which causes said gene product to be overexpressed.

4. The method of claim 3, wherein said gene is a homolog of a gene identified by the nucleic acid sequence of SEQ. ID. 10, SEQ. ID. 11 or SEQ. ID. 14.

5. The method of claim 1, wherein said method comprises administering to said individual an expression product of a homolog of SEQ. ID. 10, SEQ. ID. 11 or SEQ. ID. 14.

6. The method of claim 1, wherein said step of altering the level of an influenza resistance gene product in said individual comprises causing said gene to be under expressed, as compared to a level of expression of said gene in said individual's endogenous genome.

7. The method of claim 6, wherein said influenza resistance gene is a homolog of a gene identified by SEQ. ID 9, 12, 13, 15 or 16.

8. The method of claim 1, wherein the level of said gene product of said influenza resistance gene is reduced by providing to said individual a circulating titer of antibodies which specifically bind said gene product.

9. The method of claim 8, wherein said gene product is a homolog of an amino acid sequence identified by SEQ. ID. No 17, 20, 21, 23 or 24.

10. The method of claim 9, wherein said antibody is a monoclonal antibody generated in a host cell other than the individuals, and administered to said individual in vivo or ex vivo.

11. The method of claim 8, wherein said antibody is generated by said individual as an immune response to an immunogen with which said individual is inoculated.

12. An antibody which binds to an influenza resistance gene expression product, wherein said gene expression product is a homolog of SEQ. ID NO:17, 18, 19, 20, 21, 22, 23 or 24.

13. The antibody of claim 12, which antibody has been modified to be susceptible of administration to a mammal without inducing an immune response in said mammal.

14. The antibody of claim 12, wherein said antibody is produced by a eukaryotic host.
